SEQUENCE LISTING (24 sequences)

SEQ ID NO: 1 - protein, 5 aa, synthetic construct
SYWMN

SEQ ID NO: 2 - protein, 17 aa, synthetic construct
QIYPGDDDTNYNAKFQG

SEQ ID NO: 3 - protein, 12 aa, synthetic construct
SLMVTTGAPFDY

SEQ ID NO: 4 - protein, 11 aa, synthetic construct
KASQDVGIAVA

SEQ ID NO: 5 - protein, 7 aa, synthetic construct
YASHRGW

SEQ ID NO: 6 - protein, 9 aa, synthetic construct
QQYRSYPLT

SEQ ID NO: 7 - protein, 121 aa, synthetic construct
QVQLVQSGAEVKKPGSSVKVSCKASGYAFSSYWMNWVRQAPGQGLEWIGQIYPGDDDTNYNAKFQGRVTITVDKSTTTAYMELSSLRSEDTAVYFCAGSLMVTTGAPFDYWGQGTTVTVSS

SEQ ID NO: 8 - protein, 107 aa, synthetic construct
DIQMTQSPSSLSASVGDRVTITCKASQDVGIAVAWYQQKPGKAPKFLIYYASHRGWGVPDRFSGSGSGTDFTLTISSLQPEDFATYFCQQYRSYPLTFGQGTKLEIK

SEQ ID NO: 9 - protein, 450 aa, synthetic construct
QVQLVQSGAEVKKPGSSVKVSCKASGYAFSSYWMNWVRQAPGQGLEWIGQIYPGDDDTNYNAKFQGRVTITVDKSTTTAYMELSSLRSEDTAVYFCAGSLMVTTGAPFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 214 aa, synthetic construct
DIQMTQSPSSLSASVGDRVTITCKASQDVGIAVAWYQQKPGKAPKFLIYYASHRGWGVPDRFSGSGSGTDFTLTISSLQPEDFATYFCQQYRSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 7 aa, synthetic construct
GYAFSSY

SEQ ID NO: 12 - protein, 6 aa, synthetic construct
YPGDDD

SEQ ID NO: 13 - protein, 12 aa, synthetic construct
SLMVTTGAPFDY

SEQ ID NO: 14 - protein, 7 aa, synthetic construct
SQDVGIA

SEQ ID NO: 15 - skipped

SEQ ID NO: 16 - protein, 7 aa, synthetic construct
YRSYPLT

SEQ ID NO: 17 - protein, 8 aa, synthetic construct
GYAFSSYW

SEQ ID NO: 18 - protein, 8 aa, synthetic construct
IYPGDDDT

SEQ ID NO: 19 - protein, 14 aa, synthetic construct
AGSLMVTTGAPFDY

SEQ ID NO: 20 - protein, 6 aa, synthetic construct
QDVGIA

SEQ ID NO: 21 - protein, 9 aa, synthetic construct
QQYRSYPLT

SEQ ID NO: 22 - protein, 10 aa, synthetic construct
GYAFSSYWMN

SEQ ID NO: 23 - protein, 10 aa, synthetic construct
QIYPGDDDTN

SEQ ID NO: 24 - protein, 638 aa, Homo sapiens
MILFKQATYFISLFATVSCGCLTQLYENAFFRGGDVASMYTPNAQYCQMRCTFHPRCLLFSFLPASSINDMEKRFGCFLKDSVTGTLPKVHRTGAVSGHSLKQCGHQISACHRDIYKGVDMRGVNFNVSKVSSVEECQKRCTNNIRCQFFSYATQTFHKAEYRNNCLLKYSPGGTPTAIKVLSNVESGFSLKPCALSEIGCHMNIFQHLAFSDVDVARVLTPDAFVCRTICTYHPNCLFFTFYTNVWKIESQRNVCLLKTSESGTPSSSTPQENTISGYSLLTCKRTLPEPCHSKIYPGVDFGGEELNVTFVKGVNVCQETCTKMIRCQFFTYSLLPEDCKEEKCKCFLRLSMDGSPTRIAYGTQGSSGYSLRLCNTGDNSVCTTKTSTRIVGGTNSSWGEWPWQVSLQVKLTAQRHLCGGSLIGHQWVLTAAHCFDGLPLQDVWRIYSGILNLSDITKDTPFSQIKEIIIHQNYKVSEGNHDIALIKLQAPLNYTEFQKPICLPSKGDTSTIYTNCWVTGWGFSKEKGEIQNILQKVNIPLVTNEECQKRYQDYKITQRMVCAGYKEGGKDACKGDSGGPLVCKHNGMWRLVGITSWGEGCARREQPGVYTKVAEYMDWILEKTQSSDGKAQMQSPA

(19) United States
(12) Patent Application Publication
(10) Pub. No.: US 2024/0343829 A1
(43) Pub. Date: Oct. 17, 2024

(54) ANTI-PLASMA KALLIKREIN ANTIBODY DOSING REGIMENS FOR TREATING HEREDITARY ANGIOEDEMA

(71) Applicant: Astria Therapeutics, Inc., Boston, MA (US)

(72) Inventors: Jou-Ku Chung, Brookline, MA (US); Christopher John Morabito, Lexington, MA (US); Andrew John Nichols, Marblehead, MA (US)

(21) Appl. No.: 18/615,934

(22) Filed: Mar. 25, 2024

Related U.S. Application Data
(63) Continuation-in-part of application No. PCT/US2023/083936, filed on Dec. 13, 2023.
(60) Provisional application No. 63/597,115, filed on Nov. 8, 2023.
(60) Provisional application No. 63/518,023, filed on Aug. 7, 2023.
(60) Provisional application No. 63/498,163, filed on Apr. 25, 2023.
(60) Provisional application No. 63/491,335, filed on Mar. 21, 2023.
(60) Provisional application No. 63/446,299, filed on Feb. 16, 2023.
(60) Provisional application No. 63/482,674, filed on Feb. 1, 2023.
(60) Provisional application No. 63/387,187, filed on Dec. 13, 2022.

(51) Int. Cl. C07K 16/40; A61K 9/00; A61K 39/00; A61P 43/00

(52) U.S. Cl. CPC C07K 16/40; A61K 9/0019; A61P 43/00; A61K 2039/505; A61K 2039/545; C07K 2317/76

(57) ABSTRACT

The invention describes methods for administering anti-plasma kallikrein antibodies, e.g., dosing regimens, to treat plasma kallikrein associated disorders, including hereditary angioedema (HAE).

### CROSS REFERENCE TO RELATED APPLICATIONS

This application is a continuation-in-part of International Patent Application No. PCT/US2023/083936, filed Dec. 13, 2023, in the United States Patent and Trademark Office as Receiving Office under the Patent Cooperation Treaty, which International application designated the United States and claims priority to and the benefit of U.S. Provisional Patent Application No. 63/387,187, filed Dec. 13, 2022; U.S. Provisional Patent Application No. 63/482,674, filed Feb. 1, 2023; U.S. Provisional Patent Application No. 63/446,299, filed Feb. 16, 2023; U.S. Provisional Patent Application No. 63/491,335, filed Mar. 21, 2023; U.S. Provisional Patent Application No. 63/498,163, filed Apr. 25, 2023; U.S. Provisional Patent Application No. 63/518,023, filed Aug. 7, 2023; and U.S. Provisional Patent Application No. 63/597,115, filed Nov. 8, 2023; the contents of each of the foregoing International and U.S. provisional applications are incorporated by reference herein in their entireties.

### REFERENCE TO A SEQUENCE LISTING XML

This application contains a Sequence Listing which has been submitted electronically in XML format. The Sequence Listing XML is incorporated herein by reference. Said XML file, created on Apr. 29, 2024, is named AST-011AUSCIP_SL.xml and is 22,666 bytes in size.

### FIELD OF THE INVENTION

The invention relates to the fields of plasma kallikrein and disorders associated with plasma kallikrein, including hereditary angioedema (HAE), and to therapeutic antibodies, in particular, anti-plasma kallikrein (pKal) antibodies, and methods for treating plasma kallikrein associated disorders, including HAE, and more specifically dosing regimens for treating plasma kallikrein associated disorders by administering anti-pKal antibodies.

### BACKGROUND

Plasma kallikrein (pKal) is a serine protease and a potential drug target for different inflammatory, cardiovascular, infectious (sepsis) and oncology diseases (Sainz I. M. et al., Thromb Haemost 98, 77-83, 2007). Activation of pKal amplifies intrinsic coagulation via its feedback activation of factor XII and enhances inflammation via the production of the proinflammatory nonapeptide bradykinin. As the primary kininogenase in the circulation, plasma kallikrein is largely responsible for the generation of bradykinin in the vasculature.

Hereditary angioedema (HAE) is caused by a rare autosomal dominant disorder causing a deficiency in functional C1 esterase inhibitor (C1-INH), a serpin family protein that blocks the activity of plasma kallikrein (pKal). Insufficient inhibition of pKal results in the over production of bradykinin, a vasoactive inflammatory mediator that produces acute attacks of painful and unpredictable angioedema (swelling under the skin), as well as swelling (edema) in the face, limbs, abdomen, and airway (Zuraw B. L. et al., N Engl J Med 359, 1027-1036, 2008). Such attacks, often precipitated by unknown triggers, can have potentially-life threatening consequences.

The majority of HAE cases (Type I and Type II) are caused by mutations in the SERPING1 gene that lead to a reduction in the amount (Type I, approximately 85% of patients) or function (Type II, approximately 15% of patients) of C1-esterase inhibitor protein (C1-INH) encoded by this gene (Busse et al., 2021, J Allergy Clin Immunol Pract., 9:132-150 e133; Zuraw, 2008, N Engl J Med, 359:1027-1036). Fully functional C1-INH inhibits the activity of two key enzymes in the contact activation system, factor XIIa (FXIIa) and pKal; a deficiency in the amount or function of C1-INH results in the unregulated activity of these enzymes (Craig et al., 2012, Allergy Asthma Proc, 22, 354-361). The uninhibited activity of the contact pathway in Type I and Type II HAE leads to an unchecked and widespread surge in the production of bradykinin by plasma kallikrein, resulting in excessive and pathologic edema and pain (Zeerleder et al., 2016, Ann Med. 48 (4): 256-267).

The estimated prevalence of Type I and Type II HAE ranges from 1 in 10,000 to 1 in 50,000 with less than an estimated 8,000 patients in the United States and less than an estimated 15,000 patients in Europe (Busse et al., 2021, J Allergy Clin Immunol Pract., 9:132-150 e133; Lumry et al., 2018, Front Med (Lausanne), 5:22; Nzeako et al., Arch Intern Med., 151:2417-2429. Patients with HAE are typically diagnosed by the age of 20 with the average age of disease onset of approximately 11 years old (Christiansen et al., Clinical Pediatrics, 2016, 55 (10): 935-942). Patients typically present with symptoms to the emergency room or to a primary care physician, but limited disease awareness, although improving, often precludes referrals to allergists, immunologists, and HAE specialists for diagnosis. Clinical suspicion of Type I and II HAE can be confirmed by available blood tests. In addition to abnormalities in C1-INH level and function, plasma C4 levels are markedly reduced at all times in blood from most patients. The most common comorbidities associated with HAE include anxiety, depression, cardiovascular disease, hypertension, and hyperlipidemia (Banerji et al., 2017, N. Engl J Med, 376:717-728) and autoimmune disease, particularly systemic lupus erythematosus and thyroid disease (Sundler Bjorkman et al., 2022, Clin Transl Allergy, 12 (3): e12135). The psychiatric symptoms are not uncommon but are generally considered to be caused by the burden of the disease (Banerji et al., 2017, N. Engl J Med, 376:717-728; Lumry et al., 2018, Front Med (Lausanne), 5:22).

There are generally two treatment approaches to manage the unpredictable and recurrent edema attacks typically experienced by people with HAE: (1) on-demand, where acute treatments are administered at the onset of an attack to reduce the severity and duration of the attack, and (2) preventative or prophylactic, where treatments are taken chronically or prior to a predictive event to reduce the frequency and severity of future attacks. In the United States, the Food and Drug Administration has approved several on-demand and preventative therapies for the treatment of HAE; however, despite the existence of these therapies, many treatment strategies fail to fully prevent HAE attacks. This is primarily due to variability in patient responses to approved medications, which leads to unpredictable and incomplete attack prevention, and burdensome therapeutic regimens that require frequent administration.

For example, dosing regimens for existing therapeutics to treat plasma kallikrein associated disorders, such as HAE, whether administered orally or parenterally, can be challenging for patients. TAKHZYRO® (lanadelumab-flyo), a monoclonal anti-plasma kallikrein antibody was approved by the U.S. Food & Drug Administration in 2018 for prophylaxis to prevent HAE attacks; however, administration of 300 mg of the antibody is required every 2 weeks, and may only be appropriate for dosing up to every 4 weeks in some patients. CINRYZE® (C1-esterase inhibitor (human)), is a C1-inhibitor protein (C1-INH) isolated from purified human blood and must be administered intravenously every 3-4 days. ORLADEYO® (berotralstat), a small molecule inhibitor of plasma kallikrein, must be taken orally on a daily basis.

Therapeutic antibodies against pKal can prevent attacks, allowing HAE patients to lead more normal lives without fear that any given activity could trigger severe and potentially even lethal attacks. Maintaining efficacy, by suppressing attack frequency and severity, coupled with a reduced dosing frequency would significantly improve the lives of people living with HAE. Accordingly, dosing strategies for HAE treatments are needed that reduce dosing frequency to improve both patient compliance, ease of administration, and quality of life.

### SUMMARY OF THE INVENTION

The invention is based, in part, upon the discovery that antibodies that bind plasma kallikrein, e.g. human plasma kallikrein, can be used to treat plasma kallikrein dependent disorders, such as HAE, and that treatment of these disorders with the antibodies disclosed herein can be accomplished using a dosing regimen that permits infrequent dosing compared to the dosing regimens required by commercially available pKal antibodies and other therapies.

The methods disclosed herein achieve therapeutically effective levels of pKal inhibition over intervals as long as three months or more, allowing subjects with HAE to receive doses of the antibody at infrequent intervals, improving patient quality of life and reducing the risk of severe and potentially lethal HAE attacks for months at a time from just one dose of antibody.

The methods disclosed herein are designed to provide a safe and effective preventative treatment, dosed infrequently, to decrease the burden of disease and the burden of treatment for people with plasma kallikrein associated disorders, e.g., HAE.

The invention provides a method of reducing the number of hereditary angioedema (HAE) attacks that a subject with HAE experiences per month (monthly attack rate). The method comprises treating the subject with an anti-plasma kallikrein antibody comprising two heavy chains and two light chains, wherein each heavy chain comprises the amino acid sequence of SEQ ID NO:9 and each light chain comprises the amino acid sequence of SEQ ID NO:10, e.g., STAR-0215. According to this method, the subject is administered the antibody according to one of the following dosing regimens:
(1) administering a loading dose of 600 mg of the antibody followed by administering a maintenance dose of 300 mg of the antibody about every three months after the loading dose;
(2) administering a first dose of 600 mg of the antibody followed by administering a second dose of 600 mg of the antibody about 28 days after the first dose, and then administering 600 mg of the antibody about every six months after the second dose; or
(3) administering a dose of at least 450 mg, e.g., 450-1,200 mg, of the antibody to the subject every six months.

Treatment according to this method reduces the monthly attack rate for the subject from the subject's baseline monthly attack rate prior to initiation of the treatment by at least about 85%, e.g., by at least about 86%, about 87%, about 88%, about 89%, about 90%, about 91% about 92%, about 93%, about 94%, about 95%, about 96%, about 97%, about 98%, about 99%, or by 100%, for at least 3 months from initiation of the dosing regimen, e.g., for at least 6 months.

In some embodiments of this method, the treatment is according to dosing regimen (1) above, while in other embodiments, it is according to dosing regimen (2) above, and in yet others it is according to dosing regimen (3) above. When the treatment is according to dosing regimen (3), the dose may be, e.g., 450-900 mg, or 450-600 mg, e.g., 600 mg. In some examples of dosing regimen (3), the initial dose of at least 450 mg may be preceded by a dose that is the same as or higher than the initial dose less than 6 months before the initial dose, e.g., about 1 month before or about 3 months before administration of the initial dose of at least 450 mg.

Depending upon the circumstances, the subject may also be attack free for at least three months from the initiation of the dosing regimen, e.g., for at least six months. Administration of the antibody may be accomplished, for example, by subcutaneous administration.

The invention also provides a method of reducing the severity and/or duration of hereditary angioedema (HAE) attacks in a subject with HAE. The method comprises treating the subject with an anti-plasma kallikrein antibody comprising two heavy chains and two light chains, wherein each heavy chain comprises the amino acid sequence of SEQ ID NO:9 and each light chain comprises the amino acid sequence of SEQ ID NO:10, e.g., STAR-0215. According to this method, the subject is administered the antibody according to one of the following dosing regimens:
(1) administering a loading dose of 600 mg of the antibody followed by administering a maintenance dose of 300 mg of the antibody about every three months after the loading dose;
(2) administering a first dose of 600 mg of the antibody followed by administering a second dose of 600 mg of the antibody about 28 days after the first dose, and then administering 600 mg of the antibody about every six months after the second dose; or
(3) administering a dose of at least 450 mg, e.g., 450-1,200 mg, of the antibody to the subject every six months.

In some embodiments of this method, the treatment is according to dosing regimen (1) above, while in others, it is according to dosing regimen (2) above, and in yet others it is according to dosing regimen (3) above. When the treatment is according to dosing regimen (3), the dose may be, e.g., 450-900 mg, or 450-600 mg, e.g., 600 mg. In some examples of dosing regimen (3), the initial dose of at least 450 mg may be preceded by a dose that is the same as or higher than the initial dose less than 6 months before the initial dose, e.g., about 1 month before or about 3 months before administration of the initial dose of at least 450 mg.

Depending on the circumstances, treatment according to this method reduces the monthly rate of moderate or severe HAE attacks the subject experiences by at least 85%, e.g., about 86%, about 87%, about 88%, about 89%, about 90%, about 91%, about 92%, about 93%, about 94%, about 95%, about 96%, about 97%, about 98%, about 99%, or by 100%, for at least 3 months, e.g., at least 6 months, from initiating of the dosing regimen as compared to the monthly rate of moderate or severe HAE attacks prior to initiation of the treatment. For example, the treatment reduces the monthly rate of moderate or severe HAE attacks by at least 90% for at least 3 months, e.g. 6 months, from initiation of the dosing regimen. For example, the subject may be free of severe HAE attacks for at least 3 months from initiation of the dosing regimen, e.g., for at least 6 months. For example, the subject may be free of moderate HAE attacks for at least 3 months from initiation of the dosing regimen, e.g., for at least 6 months.

In other embodiments, this method provides a reduction in the average duration of HAE attacks the subject experiences for at least three months, e.g., for at least 6 months, from commencement of the dosing regimen as compared to a baseline average duration of HAE attacks in the subject prior to treatment.

According to this method, administration of the antibody may be accomplished, for example, by subcutaneous administration.

The invention also provides a method of reducing the need for a subject with HAE to receive on-demand therapy (rescue medication) when experiencing an HAE attack. This method comprises treating the subject with an anti-plasma kallikrein antibody comprising two heavy chains and two light chains, wherein each heavy chain comprises the amino acid sequence of SEQ ID NO:9 and each light chain comprises the amino acid sequence of SEQ ID NO: 10, e.g., STAR-0215. According to this method, the subject is administered the antibody according to one of the following dosing regimens:
(1) administering a loading dose of 600 mg of the antibody followed by administering a maintenance dose of 300 mg of the antibody about every three months after the loading dose;
(2) administering a first dose of 600 mg of the antibody followed by administering a second dose of 600 mg of the antibody about 28 days after the first dose, and then administering 600 mg of the antibody about every six months after the second dose; or
(3) administering a dose of at least 450 mg, e.g., 450-1,200 mg, of the antibody to the subject every six months.

In some embodiments of this method, the treatment is according to dosing regimen (1) above, while in others, it is according to dosing regimen (2) above, and in yet others it is according to dosing regimen (3) above. When the treatment is according to dosing regimen (3), the dose may be, e.g., 450-900 mg, or 450-600 mg, e.g., 600 mg. In some examples of dosing regimen (3), the initial dose of at least 450 mg may be preceded by a dose that is the same as or higher than the initial dose less than 6 months before the initial dose, e.g., about 1 month before or about 3 months before administration of the initial dose of at least 450 mg.

The treatment according to this method reduces the subject's monthly attack rate for which the subject receives on-demand therapy by at least 85%, e.g., by at least about 86%, about 87%, about 88%, about 89%, about 90%, about 90%, about 91%, about 92%, about 93%, about 94%, about 95%, about 96%, about 97%, about 98%, about 99%, or by 100%, compared to the subject's monthly attack rate for which the subject received on-demand therapy prior to initiation of treatment, wherein the reduction lasts for at least 3 months, e.g. for at least 6 months, from the initiation of the dosing regimen. In some circumstances, the reduction is, about 93%, or 94%, or 95%.

In some circumstances, the subject does not experience any HAE attacks requiring receipt of on-demand therapy for at least three months, e.g., for at least six months, from initiation of the dosing regimen.

According to this method, administration of the antibody may be accomplished, for example, by subcutaneous administration.

The invention also provides a method of maintaining a serum concentration of greater than about 12 μg/mL of an anti-plasma kallikrein antibody in subject with hereditary angioedema. This method comprises administering to the subject an anti-plasma kallikrein antibody comprising two heavy chains and two light chains, wherein each heavy chain comprises the amino acid sequence of SEQ ID NO:9 and each light chain comprises the amino acid sequence of SEQ ID NO:10, e.g., STAR-0215. The antibody is administered according to one of the following dosing regimens:
(1) administering a loading dose of 600 mg followed by administering a maintenance dose of 300 mg three months after the loading dose;
(2) administering a first dose of 600 mg followed by administering a second dose of 600 mg about 28 days after the first dose; or
(3) administering a dose of at least 450 mg, e.g., 450-1,200 mg, of the antibody to the subject every six months.

In some embodiments of this method, the treatment is according to dosing regimen (1) above, while in others, it is according to dosing regimen (2) above, and in yet others it is according to dosing regimen (3) above. When the treatment is according to dosing regimen (3), the dose may be, e.g., 450-900 mg, or 450-600 mg, e.g., 600 mg. In some examples of dosing regimen (3), the initial dose of at least 450 mg may be preceded by a dose that is the same as or higher than the initial dose less than 6 months before the initial dose, e.g., about 1 month before or about 3 months before administration of the initial dose of at least 450 mg.

According to the method, the serum concentration is exceeded for at least three months, e.g., at least 6 months, from administering the loading or first dose. For example, in the case of dosing regimen (1), the serum concentration is exceeded for at least three months from administering the loading dose, or for at least three months from administering a maintenance dose. For example, in the case of dosing regimen (2), the serum concentration is exceeded for at least 6 months from administration of the first dose, or for at least 6 months from administering the second dose, or a subsequent 600 mg dose. For example, in the case of dosing regimen (3), the serum concentration is exceeded for at least 6 months from administration of an initial dose, or for at least 6 months from each dose.

According to this method, the antibody may be administered to the subject via subcutaneous injection.

### BRIEF DESCRIPTION OF THE FIGURES

FIG. 1 is a schematic of the biochemical and cellular processes leading to hereditary angioedema caused by a deficiency in the amount or function of C1-INH. Abbreviations: BIR GPCR=G-protein-coupled receptor bradykinin receptor 1; B2R GPCR=G-protein-coupled receptor bradykinin receptor 2; C1-INH=C1 esterase inhibitor; des-R9-BK=des-Arg (9)-bradykinin; FXII=factor XII; FXIIa=factor XIIa; HMWK=high-molecular-weight kininogen

FIGS. 2A-C show that STAR-0215 inhibits plasma kallikrein-mediated cleavage of HMWK in cynomolgus monkeys. FIGS. 2A and 2C are Western blots which show intact and cleaved HMWK (the endogenous pKal substrate). Controls HQC, MQC, and LQC with approximately 99, 67, and 18% cHMWK, respectively are shown on the right side of each blot. FIG. 2A shows Western blots of intact HMWK and cHMWK from control animals and animals dosed with STAR-0215 at 10, 30, and 100 mg/kg up to day 43. FIG. 2C shows Western blots from control and animals dosed with STAR-0215 (100 mg/kg) up to day 127. FIG. 2B is a line graph depicting data from the Western blot analysis and shows percentage (%) cHMWK±SD (n=3) over the study period.

FIG. 3 is a graph of the results of a pharmacokinetic model predicting the target concentration of STAR-0215 required to produce long-term inhibition of pKal. The graph shows the predicted serum concentration of STAR-0215 in μg/mL over time. The shaded area shows the levels of plasma kallikrein in patients during HAE attacks and is the target concentration that is expected to completely inhibit plasma kallikrein in patients during HAE attacks. The highest line on the graph is a 600 mg dose, the middle line is 300 mg, and the lowest is 100 mg. The modeling results show that a target concentration of STAR-0215 can be achieved with a single dose ≥100 mg. Target concentration (shown in green) that is expected to completely inhibit pKal in patients during HAE attacks.

FIG. 4 is a schematic showing the overview of the Phase 1a study design to assess the safety, tolerability, pharmacokinetics, pharmacodynamics, and immunogenicity of STAR-0215 in healthy adult subjects.

FIG. 5 is a series of bar graphs showing the level of pKal activation and inhibition by STAR-0215 as determined by the level of cleaved high molecular weight kininogen (cHMWK) as a percentage of cHMWK+intact HMWK over time in plasma samples from study cohorts after ex vivo activation of the plasma by Factor XIIa. The data is shown from left to right for placebo, the 100 mg cohort, the 300 mg cohort, and the 600 mg cohort, and provides the level of cleaved high molecular weight kininogen (cHMWK) as a percentage of cHMWK+intact HMWK over time from day 1 through day 84 for each cohort. Data is represented as mean+SD % cHMWK; Unless otherwise noted, n=6 subjects for each timepoint in each dose group. 100 mg, n=7 at all timepoints; 600 mg Day 56 and Day 84, n=5. The shaded area at the bottom of each graph shows the mean level of cHMWK in unactivated plasma samples (12%).

FIG. 6 is a series of bar graphs showing the level of pKal activation as determined by the level of cleaved high molecular weight (cHMWK) in plasma samples taken from study cohorts pre-dose and on day 2 and day 6 after STAR-0215 administration when activated ex vivo by Factor XIIa. The shaded area at the bottom of the graph shows the level of cHMWK in unactivated plasma samples. Data is represented as mean+SD. Pre=predose. Statistics calculated by one-way ANOVA relative to predose for each dose level. *=p<0.05; **=p<0.005; ***=p<0.0005

FIGS. 7A-B are graphs showing the level of pKal activation as determined by the level of cleavage of high molecular weight (cHMWK) in plasma samples taken from study cohorts. FIG. 7A is a bar graph showing the level of pKal activation from predose through day 84 for the 300 mg and 600 mg cohorts after STAR-0215 administration when activated ex vivo by Factor XIIa. Data represented as mean+SD: STAR-0215 Statistics calculated by a mixed effects model with Dunnett's correction for multiple comparison relative to predose for STAR-0215;*=p<0.05; **=p<0.01; ***=p<0.0002. The shaded area at the bottom of each graph shows the level of cHMWK in unactivated plasma samples. A comparison to similar parameters for the anti-pKal antibody lanadelumab is provided in the right panel as reported in Chyung et al. (Ann Allergy Asthma Immunol, 2014, 113:460-466). FIG. 7B is a line graph showing the level of pKal activation from day 1 through day 224 in the 300 mg SC and 600 mg SC STAR-0215 cohorts and through day 84 in the 1,200 mg SC STAR-0215 cohort after administration when activated by ex vivo by Factor XIIa, as obtained by Western blot. The data are mean+/−SD, p<0.05 using a t test.

FIG. 8 is a line graphs showing the serum levels of STAR-0215 in cohorts of healthy study subjects receiving one 100 mg, 300 mg, or 600 mg bolus subcutaneous injection of STAR-0215. The serum levels on day 1 through day 84 for each cohort are provided. The dotted horizontal line shows the concentration threshold of 12 μg/mL (80 nM) associated with clinical benefit. The mean (SD) concentration of STAR-0215 over time is shown.

FIGS. 9A-B are line graphs showing the serum levels of STAR-0215 in cohorts of healthy study subjects receiving 300 mg (▪) nd 600 mg (▴) bolus subcutaneous injections of STAR-0215. FIG. 9A shows the levels on day 1 and through day 84 for the 300 mg cohort and for day 1 through day 56 for the 600 mg cohort (as data was only available through day 56). FIG. 9B provides the levels for the 300 mg cohort as shown in FIG. 9A, but also includes the levels for the 600 mg cohort through day 84. The levels in each graph are compared to lanadelumab (♦) as reported by Chyung et al. (Ann Allergy Asthma Immunol, 2014, 113:460-466). PK comparisons of t ½, Tₘₐₓ, Cₘₐₓ, and CL are provided in the tables in each figure; CL was not available for the 600 mg cohort as shown in FIG. 9B as none of the cohort members met the calculation criteria (*), and for the 300 mg cohort as shown in FIG. 9B, only one subject met the CL calculation criteria and had a reportable CL (**). T1/2=half-life; Tₘₐₓ=time to maximum concentration; Cₘₐₓ=maximum concentration; CL=clearance

FIG. 10 is a graph of the results of a pharmacokinetic model predicting the target concentration of STAR-0215 required to produce long-term inhibition of pKal. The graph shows the predicted serum concentration of STAR-0215 in μg/mL over time for doses of 300 mg, 450 mg, 600 mg, and 900 mg. The dotted horizontal line shows a target level of 12 μg/mL (80 nM) believed necessary for pKal inhibition sufficient to prevent HAE attacks. The modeling results show that a concentration of greater than 12 μg/mL of STAR-0215 can be maintained over 84 days (3 months) with a single dose ≥450 mg.

FIGS. 11A-B are graphs showing a simulated human PK profile based on various dosing regimens of STAR-0215. FIG. 11A shows the simulated human PK profile based on a 600 mg loading dose at day 0 with 300 mg maintenance doses provided in 84 day (3 month) intervals after the initial loading dose, while FIG. 11B shows the simulated human PK profile based on a 600 mg dose at day 1, again at day 28, and every 6 months thereafter. The horizontal dotted line represents the threshold concentration of 80 nM (12 μg/mL) believed necessary for pKal inhibition sufficient to prevent HAE attacks. As shown in FIG. 11A, the dosing regimen according to the model simulation maintains a steady state concentration of STAR-0215 greater than 80 nM (12 μg/mL) which is 27 μg/mL. In FIG. 11B, the dosing regimen according to the model simulation maintains a steady state concentration of STAR-0215 greater than 80 nM (12 μg/mL) which is 16 μg/mL. The results suggest that STAR-0215 may provide a clinical benefit with infrequent dosing, e.g., every three months or even every six months.

FIG. 12 is a schematic showing the overview of the Phase 1b/2 study design to assess the safety, tolerability, clinical activity, pharmacokinetics, pharmacodynamics, and immunogenicity of STAR-0215 in participants with Hereditary Angioedema (HAE). The study will enroll three cohorts. Cohort 1 will receive a single dose of STAR-0215 on Day 1, Cohort 2 will receive doses of STAR-0215 on Days 1 and 84, and Cohort 3 will receive doses of STAR-0215 on Days 1 and 28.

FIG. 13 shows Western blot images of intact HMWK and cleaved HMWK from representative subjects in the 100 mg, 300 mg, and 600 mg cohorts from pre-dose through day 84 after administration when activated ex vivo by Factor XIIa. The images show that STAR-0215 inhibits ex-vivo Factor XIIa-induced cleavage of plasma HMWK after a single subcutaneous dose and is indicative of a decrease in the level of pKal activation compared to placebo.

FIG. 14 is a graph showing the percent change in cHMWK from pre-dose levels in FXIIa activated samples for all members of each cohort. Significant inhibition of cHMWK at all post-dose time points was observed for the 300 mg and 600 mg cohorts. N=6 for all cohorts. The data are mean±SD with *=p<0.05 from pre-dose at indicated doses and timepoints; non-significant (ns) difference at all timepoints for placebo. The statistical test was a 2-Way ANOVA with Dunnett's test for multiple comparisons.

FIG. 15 is a graph showing % inhibition of plasma kallikrein activity based on an orthogonal chromogenic reporter-substrate (PFR-pNA) assay and confirms dose-responsive, robust and long-lasting inhibition of plasma kallikrein activity after single dose of STAR-0215. N=6 for all cohorts. The data are mean±SD with *=p<0.05 from pre-dose at indicated doses and timepoints; non-significant (ns) difference at all timepoints for placebo. The statistical test was a 2-Way ANOVA with Dunnett's test for multiple comparisons.

FIG. 16 shows two graphs generated by a population PK model predicting the pharmacokinetic duration of a 600 mg dose of STAR-0215 administered at Day 1 and again at Day 28 (left hand graph), or at Day 1 and Day 14 (right hand graph) over time. The graphs show that predicted serum levels of STAR-0215 will fall below a target Cₘᵢₙ of 12 μg/mL around 7 months (lower dotted horizontal line), whereas predicted serum levels of STAR-0215 will fall below a target Cₘᵢₙ of 25 μg/mL (upper dotted horizontal line) around 5 months. The Day 1/Day 14 dosing has an increased Cₘₐₓ compared to the Day 1/Day 28 dosing.

FIGS. 17A-C are graphs generated by a population PK model predicting the pharmacokinetic duration of various dosing regimens administered to the cohorts of the Phase 1b/2 clinical study described in Example 5. FIG. 17A shows the simulated human PK profile of study cohort 1 based on a 450 mg on day 1; FIG. 17B shows the simulated human PK profile of study cohort 2 based on a 600 mg dose at day 1, and a 300 mg dose at day 84; and FIG. 17C shows the simulated human PK profile of study cohort 3 based on a 600 mg dose administered at day 1 and again at day 28. The horizontal dotted line represents the threshold concentration of 80 nM (12 μg/mL) believed necessary for pKal inhibition sufficient to prevent HAE attacks. The population pK model predicts that for cohort 1, the threshold is maintained for at least 3 months; for cohort 2, the threshold is maintained at least 6 months from the first dose, or for at least three months after each dose; and for cohort three, the threshold is maintained for at least 6 months from the first dose.

FIG. 18 is a schematic drawing of the quantitative systems pharmacology (QSP) model described in Example 3 and shows the interaction of STAR-0215 with the plasma kallikrein-kininogen pathway.

FIG. 19 shows the results from the QSP model simulations described in Example 3, and shows the HAE attack frequency per month predicted by the model for the virtual cohort. The top bar graph shows the results of a simulation via the QSP model where Cohort 1 (n=500) received a dosing regimen of a single subcutaneous loading dose of 600 mg of STAR-0215 on Day 1, followed by subcutaneous maintenance doses of 300 mg of STAR-0215 every 3 months (84 days) thereafter, according to one simulation via the QSP Model. The bottom bar graph shows the results of a simulation via the QSP model where Cohort 2 (n=500) received a dosing regimen of a subcutaneous dose of 600 mg STAR-0215 on Day 1, again on Day 28, and then every 6 months thereafter (every 168 days). The results of each simulation extend to 24 months from the first dose and are shown as the number of acute HAE attacks per month.

FIGS. 20A-G are graphs showing the results of simulations using the pharmacokinetic model described in Example 2. Various dosing regimens of STAR-0215 were modeled; median blood concentrations are provided. FIG. 20A shows the simulated STAR-0215 blood concentration (μg/mL) over time based on administering a loading dose of 600 mg followed by 300 mg maintenance doses at 3 month intervals thereafter. FIG. 20B shows the simulated STAR-0215 blood concentration (μg/mL) over time (336 days) based on administering 600 mg on Day 1, followed by 600 mg on Day 28 (i.e., one month after the first dose), and then subsequent doses every 6 months (or put another way, 600 mg every six months starting 1 month after a 600 mg loading dose), where C_{trough} is 25.1 μg/mL which is the concentration of STAR-0215 at the end of the dosing interval. FIG. 20C shows the simulated STAR-0215 blood concentration (μg/mL) over time based on administering a dose of 1,200 mg at 6 month intervals. FIG. 20D shows the simulated STAR-0215 blood concentration (μg/mL) over time based on administering a dose of 1,200 mg followed by 900 mg every 6 months thereafter. FIG. 20E shows the simulated STAR-0215 blood concentration (μg/mL) over time based on administering a dose of 1,200 mg, followed by 1,200 mg three months later, followed by 1,200 mg every 12 months thereafter. The horizontal dotted line represents the threshold concentration of 80 nM (12 μg/mL) believed necessary for pKal inhibition sufficient to prevent HAE attacks. FIG. 20F shows the results in FIGS. 20A-D superimposed on one another. FIG. 20G shows the results of the simulation in FIG. 20B, but extended through 24 months, where C_{trough} is 27.0 μg/mL which is the concentration of STAR-0215 at the end of the dosing interval. The population pK model predicts that each of the dosing regimens shown in FIGS. 20A-G has potential to maintain the required threshold necessary for pKal inhibition to prevent HAE attacks in HAE patients.

FIG. 21 is a graph showing the mean concentration of blood levels of STAR-0215 in study cohorts from the Phase 1a clinical study cohorts described in Example 4 at various time points. At 84 days from administration, the 1,200 mg cohort had a mean blood level of 72.7 μg/mL, and 28 days from administration, the 600 mg i.v. cohort, had a mean blood level of 75.4 μg/mL.

FIG. 22 is a graph showing the concentration (μg/mL) of STAR-0215 through day 224 in each of the 100 mg, 300 mg and 600 mg cohorts receiving STAR-0215 subcutaneously, through day 84 for the 600 mg cohort receiving STAR-0215 intravenously, and through day 112 for the 1,200 mg cohort receiving STAR-0215 subcutaneously. The dotted horizontal line indicates the concentration threshold of 12 μg/mL (80 nM) associated with potential clinical benefit. For the 100 mg cohort, n=7, except for Day 140 onwards where n=6. For the 600 mg cohort, n=6, except for Day 56 onwards where n=5. For the 1,200 mg cohort, n=6, except for the 12h timepoint where n=5.

FIG. 23 is a graph showing % inhibition of plasma kallikrein activity based on an orthogonal chromogenic reporter-substrate (PFR-pNA) assay for the 300 mg subcutaneous (SC), 600 mg SC, and 1,200 mg SC cohorts through day 224. % inhibition was determined relative to predose timepoints. A statistically significant inhibition of ex vivo FXIIa-activated plasma kallikrein activity was observed through day 84 after single doses of 300 mg and 600 mg, and through day 224 after a single dose of 1,200 mg SC. N=6 for all cohorts, with the exception of the 600 mg SC cohort where N=5 from Day 56 onwards. The data are mean #SD with *=p<0.005 from pre-dose at indicated doses and timepoints; non-significant (ns) difference at all timepoints for placebo. The statistical test was a t-test of the change from baseline equal to 0 at each timepoint without multiplicity adjustment.

FIG. 24 is a table providing the baseline characteristics for the subjects enrolled in each of Cohort 1, Cohort 2, and Cohort 3 of the study described in Example 5.

FIG. 25 is a swimmer's lane plot of the number, duration, and severity of HAE attacks for every subject in each of Cohorts 1, 2, and 3. Shaded horizontal bars are the duration of follow-up for each subject at the time of data cut-off. Severity of attacks are depicted by the color of the vertical bar (mild=light gray; moderate=black; severe=black+*). Vertical lines indicated efficacy analyses at Day 82 (3 months) and Day 168 (6 months). Duration of HAE attacks correlates with the thickness of each vertical bar. Cohort 1 received 450 mg of STAR-0215; Cohort 2 received 600 mg STAR-0215, followed by 300 mg after 84 days; and Cohort 3 received 600 mg STAR-0215, followed by 600 mg after 28 days.

FIG. 26 is a bar graph showing the mean time-normalized monthly HAE attack rate for each of Cohorts 1, 2, and 3 of Example 5 during the run-in period (baseline), and for 0-3 and 0-6 months post-treatment, as well as the change from baseline in each cohort. Data is provided only for participants with complete follow-up through 3 and 6 months.

FIG. 27 is a bar graph showing the percentage of attack-free participants in each cohort at 3 months from commencement of treatment. As the time of the data cut off, all 4 subjects in Cohort 1, all 6 subjects from Cohort 2, but only 4 subjects from Cohort 3 had made it to the 3 month follow up.

FIG. 28 is a bar graph showing initial data for the mean monthly attack rates for moderate and severe HAE attacks only in subjects in Cohorts 1, 2, and 3 at baseline, over the period of 0-3 months from commencement of treatment, and over the period of 0-6 months from commencement of treatment. The change in attack rate for moderate and severe HAE attacks is also shown. At 0-3 and 0-6 months, participants with complete follow-up through 3 and 6 months are shown. In Cohort 1 (N=4), 4 participants had follow-up for 3 and 6 months. In Cohort 2 (N=6), 6 participants had 3 months and 3 participants had 6 months follow-up. In Cohort 3 (N=6), 4 participants had 3 months follow-up and no participants have yet achieved 6 months follow-up.

FIG. 29 is a bar graph showing initial data regarding the change in mean monthly HAE attacks requiring rescue medication in each of Cohorts 1, 2, and 3 at baseline, for 0-3 months from commencing treatment, and for 0-6 months from commencing treatment. Only HAE attacks requiring rescue medication are shown. At 0-3 and 0-6 months, participants with complete follow-up through 3 and 6 months are shown. In Cohort 1 (N=4), 4 participants had follow-up for 3 and 6 months. In Cohort 2 (N=6), 6 participants had 3 months and 3 participants had 6 months follow-up. In Cohort 3 (N=6), 4 participants had 3 months follow-up and no participants have yet achieved 6 months follow-up.

FIG. 30 is a line graph showing the average concentration of STAR-0215 in μg/mL for each of Cohort 1 (open circle), Cohort 2 (open square), and Cohort 3 (open triangle) over time. Data for Cohort 3 is available only to 3 months (84 days). The data are presented as mean+/−SD.

FIG. 31 is a line graph showing the % change from baseline of cleaved High Molecular Weight Kininogen (cHMWK) levels for each of Cohort 1 (open circle), Cohort 2 (open square), and Cohort 3 (open triangle) over time. Data for Cohort 3 is available only to 3 months (84 days). % cHMWK was determined by western blot assay from unactivated plasma samples. The data presented are the median interquartile range.

### DETAILED DESCRIPTION OF THE INVENTION

The invention provides methods and compositions for treating plasma kallikrein associated disorders, e.g., hereditary angioedema (HAE). The invention is based, in part, upon the discovery that antibodies that bind plasma kallikrein, e.g. human plasma kallikrein, can be used to treat plasma kallikrein dependent disorders, such as HAE, and that the treatment of these disorders with the antibodies disclosed herein can be accomplished using a dosing regimen that permits infrequent dosing compared to the dosing regimens required by commercially available pKal antibodies and other therapies.

The methods disclosed herein achieve therapeutically effective levels of pKal inhibition over intervals as long as three months or more, allowing subjects with HAE to receive doses of the antibody at infrequent intervals, improving patient quality of life while reducing the likelihood of severe and potentially lethal HAE attacks for months at a time from just one dose of antibody.

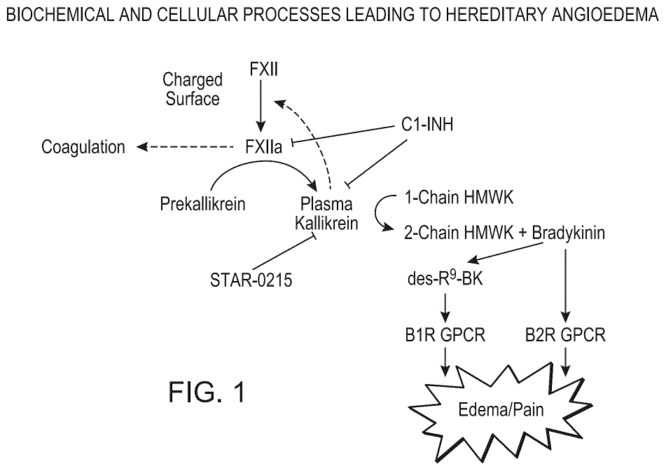

As shown in FIG. 1, HAE cases (Type I and Type II) can be caused by deficiencies in C1-INH, resulting in uncontrolled activation of plasma kallikrein, which leads to overproduction of bradykinin, a key mediator of vasodilation, angioedema, and swelling in the face, limbs, abdomen, and airway, which characterize HAE attacks (Zuraw et al., 2008, Expert Opin Investig Drugs, 17:697-706). Inhibition of plasma kallikrein by the anti-plasma kallikrein antibodies disclosed herein reduces cleavage of high molecular weight kininogen (HMWK) and the formation of bradykinin, thereby preventing the characteristic angioedema seen in HAE patients.

The methods of invention are designed to provide a safe and effective preventative treatment, dosed infrequently, to decrease the burden of disease and the burden of treatment for people with plasma kallikrein associated disorders, e.g., HAE.

### 1. Definitions

To facilitate an understanding of the present invention, a number of terms and phrases are defined below.

As used herein, the term “antibody,” e.g., an anti-plasma kallikrein antibody, refers to a full length or intact antibody which contains two immunoglobulin heavy chains, each associated with an immunoglobulin light chain. The antibody is bivalent, but each binding site binds the same target, e.g., plasma kallikrein.

As used herein, the term “chronic administration,” refers to continuous, regular, long-term administration, i.e., periodic administration of a drug, e.g., a plasma kallikrein antibody, such as STAR-0215, without substantial interruption. For example, to treat a plasma kallikrein associated disorder, such as HAE, chronic administration of a drug may occur daily, twice weekly, weekly, every two weeks, every month, every two months, every three months, every four months, every five months, every six months or more, or combinations thereof, for a period of months or years to achieve long term disease control in a subject.

As used herein, the term “complementarity determining regions” or “CDRs” refers to the immunoglobulin hypervariable domains that determine specific antibody binding. CDRs are located within four framework regions (FRs). The CDRs are usually aligned by the framework regions, enabling binding to a specific epitope. In general, from N-terminal to C-terminal, both light and heavy chain variable domains comprise FR1, CDR1, FR2, CDR2, FR3, CDR3 and FR4. As used herein, “HCDR1,” HCDR2″, and “HCDR3” refer to CDR1, 2, or 3, respectively, of the heavy chain variable region, and “LCDR1,” LCDR2″, and “LCDR3” refer to CDR1, 2, or 3 respectively, of the light chain variable region.

The assignment of amino acids to each CDR is, generally, in accordance with the definitions of Kabat (see, e.g., Sequences of Proteins of Immunological Interest, Kabat, et al.; National Institutes of Health, Bethesda, Md.; 5^{th} ed.; NIH Publ. No. 91-3242 (1991); Kabat (1978) Adv. Prot. Chem. 32:1-75; Kabat, et al., (1977) J. Biol. Chem., 252:6609-6616) or the definitions of Chothia (see, e.g., Chothia, et al., (1987) J Mol. Biol. 196:901-917 or Chothia, et al., (1989) Nature, 342:878-883), although alternative definitions are known in the art, such as IMGT (See e.g., Lefranc (2005), Nucl. Acids Res., 33, D593-D597; Lefranc et al., (2003), Dev. Comp. Immunol., 27, 55-77; Lefranc et al., (2005), Dev. Comp. Immunol., 29, 185-203 (2005); Lefranc et al., (2005), Dev. Comp. Immunol., 2005, 29, 917-938) or AbM (see, e.g., Martin, et al., (1989) Proc. Natl Acad. Sci. USA, 86, 9268-9272; Pedersen et al., (1992), Immunomethods, 1, 126; Rees et al., (1996) In Sternberg M. J. E. (ed.), Protein Structure Prediction. Oxford University Press, Oxford, 141-172). In addition to the Chothia and Kabat numbering schemes described above, the CDRs can be determined using other approaches known in the art, including for example, the Martin numbering scheme, the Gelfand numbering scheme, the IMGT numbering scheme, the Honneger numbering scheme, and the ABM numbering scheme (see, e.g., Dondelinger et al. (October 2018) Frontiers Immunol. 8, 2278, 1-15).

The term “disorder” refers to and is used interchangeably with, the terms “disease,” “condition,” or “illness.”

As used herein, the term “hereditary angioedema” or “HAE” refers to the disease or disorder caused by a genetic deficiency or dysfunction of the C1-inhibitor protein, for example, the human C1-inhibitor protein (C1-INH). HAE is generally characterized by excess pKal activation and bradykinin release. Type I and Type II HAE are both caused by a genetic mutation in the SERPING1 gene which codes for C1-IHN, while Type I results in a deficiency of C1-INH, whereas Type II results in dysfunctional C1-INH. “HAE” as described herein also includes Type III HAE in which C1-INH levels and function are normal, but where patients experience similar attacks of painful edema associated with pregnancy or estrogen intake (e.g., via oral contraceptives). Type III HAE may involve mutations in Factor XII, but not in all cases. In all types of HAE, unknown triggers cause painful edema (swelling) in the skin and/or mucosa, such as in the face, larynx, hands, feet, gastrointestinal tract and/or genitals. In some embodiments, HAE refers only to Type I and Type II HAE.

As used herein, the term “human plasma kallikrein” or “human pKal” refers to residues 391-638 shown in bold below of the human pre-Kallikrein protein (UNIPROT P03952, KLKB1) which has the following amino acid sequence:

(SEQ ID NO: 24)
MILFKQATYF ISLFATVSCG CLTQLYENAF FRGGDVASMY
TPNAQYCQMR CTFHPRCLLF SFLPASSIND MEKRFGCFLK
DSVTGTLPKV HRTGAVSGHS LKQCGHQISA CHRDIYKGVD
MRGVNFNVSK VSSVEECQKR CTNNIRCQFF SYATQTFHKA
EYRNNCLLKY SPGGTPTAIK VLSNVESGFS LKPCALSEIG
CHMNIFQHLA FSDVDVARVL TPDAFVCRTI CTYHPNCLFF
TFYTNVWKIE SQRNVCLLKT SESGTPSSST PQENTISGYS
LLTCKRTLPE PCHSKIYPGV DFGGEELNVT FVKGVNVCQE
TCTKMIRCQF FTYSLLPEDC KEEKCKCFLR LSMDGSPTRI
AYGTQGSSGY SLRLCNTGDN SVCTTKTSTR IVGGINSSWG
EWPWQVSLQV KLTAQRHLCG GSLIGHQWVL TAAHCFDGLP
LQDVWRIYSG ILNLSDITKD TPFSQIKEII IHQNYKVSEG
NHDIALIKLQ APLNYTEFQK PICLPSKGDT STIYTNCWVT
GWGFSKEKGE IQNILQKVNI PLVTNEECQK RYQDYKITQR
MVCAGYKEGG KDACKGDSGG PLVCKHNGMW RLVGITSWGE
GCARREQPGV YTKVAEYMDW ILEKTQSSDG KAQMQSPA.

The mature form of human pre-Kallikrein includes a signal peptide sequence of amino acid residues 1-19, which can be cleaved during its post-translation processing. Accordingly, the mature form of human prekallikrein comprises resides 20-638 of SEQ ID NO:24. Factor XIIa cleaves human pre-Kallikrein at R390/1391 to produce human plasma Kallikrein (pKal).

As used herein, the term “loading dose” refers to an initial higher dose or doses or dosing regimen of a drug, e.g., an anti-plasma kallikrein antibody, that is administered to a subject at the beginning of a course of treatment. The loading dose may achieve a blood concentration of the drug necessary for a therapeutic effect of the drug more rapidly than a lower dose. A loading dose may also permit a steady state concentration of the drug, e.g., the anti-plasma kallikrein antibodies disclosed herein, to be achieved more rapidly after administration of a maintenance dose. For example, the loading dose may be used to achieve a certain blood concentration (e.g., as measured in serum or plasma) of the drug, e.g., an anti-plasma kallikrein antibody, necessary for therapeutic efficacy of the drug, e.g., a blood concentration necessary to inhibit plasma kallikrein in a subject suffering from a plasma kallikrein dependent disorder, e.g., HAE.

As used herein, the term “maintenance dose” refers to dose of a drug, e.g., an anti-plasma kallikrein antibody, that is typically lower than and administered subsequent to the loading dose for the drug. The maintenance dose may be administered to a subject to maintain a steady state concentration of the drug and to maintain the therapeutic effect of the drug achieved by a loading dose. For example, the maintenance dose may be administered to maintain over a period of time a desired blood concentration (e.g., as measured in serum or plasma) of the drug initially achieved by a loading dose. For example, the maintenance dose is administered to maintain a blood concentration (e.g., as measured in serum or plasma) of a drug, e.g., an anti-plasma kallikrein antibody, in a subject over a period of time, in order to inhibit plasma kallikrein in a subject suffering from a plasma kallikrein dependent disorder, e.g., HAE. Maintenance doses may be periodically administered after the loading dose at fixed intervals.

As used herein, the term “month” refers to a period of at least 28 days, for example, 28 days or 4 weeks. In certain embodiments, then a month is 28 days. Accordingly, then, for example, three months is 84 days or 12 weeks, and six months is 168 days or 24 weeks. The term “about” with respect to a month refers to a variation of +10%. Accordingly, for example, the term “about one month” means 28 days±10%, i.e., 28 days #2.8 days, which also includes 28 days±2 days, and the term “about three months” refers to a period of 84 days #10%, i.e., +8.4 days, which also includes 84 days (12 weeks)+1 week.

As used herein “periodic administration” or “administered periodically” refers to administration with an interval between doses. For example, periodic administration includes administration at fixed intervals (e.g., weekly, monthly, every two months, every three months etc.) that may be recurring.

As used herein, the term “pharmaceutical composition” refers to the combination of an active agent, e.g., an antibody, such as an anti-plasma kallikrein antibody described herein, with a carrier, inert or active, making the composition especially suitable for diagnostic or therapeutic use in vivo or ex vivo.

As used herein, “pharmaceutically acceptable” or “pharmacologically acceptable” refers to molecular entities, compounds, compositions, materials and/or dosage forms that do not produce an adverse, allergic or other untoward reaction when administered to an animal, or to a human, as appropriate. For human administration, preparations should meet sterility, pyrogenicity, and general safety and purity standards as required by relevant regulatory authorities, e.g., the USFDA Office of Biologics standards. “Pharmaceutically acceptable” and “pharmacologically acceptable” can mean approved or approvable by a regulatory agency of the federal or a state government or the corresponding agency in countries other than the United States, or that is listed in the U.S. Pharmacopoeia or other generally recognized pharmacopoeia for use in animals, and more particularly, in humans.

The term, “pharmaceutically acceptable carrier” includes any and all solvents, excipients, fillers, dispersion media, coatings, antibacterial and antifungal agents, isotonic and absorption delaying agents and the like involved in carrying or transporting a pharmaceutical agent from one organ, or portion of the body, to another organ, or portion of the body. Except insofar as any conventional media or agent is incompatible with the active ingredient, its use in the therapeutic compositions is contemplated. Supplementary active ingredients can also be incorporated into the compositions. For examples of carriers, stabilizers and adjuvants, see Remington's Pharmaceutical Sciences, 18th ed. (Mack Publishing Company, 1990).

As used herein, “pharmaceutically acceptable excipient” refers to a substance that aids the administration of an active agent, e.g., the anti-plasma kallikrein antibodies disclosed herein, to and/or absorption by a subject without causing a significant adverse toxicological effect on the patient. Non-limiting examples of pharmaceutically acceptable excipients include water, NaCl, normal saline solutions, such as a phosphate buffered saline solution, emulsions (e.g., such as an oil/water or water/oil emulsions), lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, dextrose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compositions disclosed herein. For examples of excipients, see Remington's Pharmaceutical Sciences, 18th ed. (Mack Publishing Company, 1990).

As used herein, “plasma kallikrein associated disorder” refers to conditions that are caused by the function or aberrant expression of plasma kallikrein. These conditions include, but are not limited to, hereditary angioedema (including Type I, II, or III), bradykinin dependent edema, diabetic macular edema, retinal edema, Factor XII-associated cold autoinflammatory syndrome (FACAS), rheumatoid arthritis, gout, intestinal bowel disease, oral mucositis, neuropathic pain, inflammatory pain, spinal stenosis-degenerative spine disease, arterial or venous thrombosis, post-operative ileus, aortic aneurysm, osteoarthritis, vasculitis, edema, cerebral edema, pulmonary embolism, stroke, clotting induced by ventricular assistance devices or stents, head trauma or peri-tumor brain edema, sepsis, acute middle cerebral artery (MCA) ischemic event (stroke), restenosis (e.g., after angioplasty), systemic lupus erythematosus nephritis, Alzheimer's disease and burn injury.

As used herein, the term “STAR-0215” or “STAR-215” refers to an anti-plasma kallikrein antibody having two heavy chains and two light chains. Each heavy chain has the amino acid sequence of SEQ ID NO:9 and each light chain has the amino acid sequence of SEQ ID NO:10. STAR-0215 is a humanized IgG1 kappa light chain monoclonal antibody.

As used herein, the terms “subject” and “patient” are used interchangeably and refer to an organism to be treated by the methods and compositions of the present invention. Such organisms are preferably a mammal (e.g., human, mouse, rat, guinea pig, dog, cat, horse, cow, pig, or non-human primate, such as a monkey, chimpanzee, baboon, and rhesus). More preferably, the subject or patient is a human. A subject that is “in need thereof” refers to a subject that has been identified as requiring treatment for the condition, e.g., a plasma kallikrein dependent disorder, such as HAE, that is to be treated and is treated with the specific intent of treating such condition. In some embodiments, a subject is an adult human subject, i.e., 18 years of age or older. In some embodiments, a subject is a pediatric human subject, i.e., ages 0-17 years of age. In some embodiments, the pediatric human subject is 6-17 years of age or 12-17 years of age. In some embodiments, the pediatric human subject is one, two, three, four, five, six, seven, eight, nine, ten, eleven, twelve, thirteen, fourteen, fifteen, or sixteen years of age, or older, or seventeen years of age.

As used herein, the terms “therapeutically effective amount”, “therapeutically effective dose” and “effective amount” refer to an amount of pharmaceutically active agent, e.g., an anti-plasma kallikrein antibody, that, when administered alone (or in combination with an additional therapeutic agent) to a cell, tissue, or subject, is effective to cause a measurable improvement in one or more symptoms of a disease or condition or the progression of such disease or condition, e.g. hereditary angioedema (HAE) or another plasma kallikrein dependent disorder. A “therapeutically effective dose” further refers to that amount of the antibody sufficient to result in at least partial amelioration of symptoms, e.g., treatment, healing, prevention or amelioration of the relevant medical condition, e.g., HAE or another plasma kallikrein dependent disorder, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. An effective amount of a therapeutic may result in an improvement of a diagnostic measure or parameter by at least 10%; 20%; 30%; 40%, or 50%. An effective amount can also result in an improvement in a subjective measure in cases where subjective measures are used to assess disease severity. In some embodiments, an amount is a therapeutically effective amount if it is an amount that can be used to treat or prevent symptoms of a plasma kallikrein dependent disorder, e.g., swelling/edema. For example, a therapeutically effective amount of an anti-plasma kallikrein antibody disclosed herein may reduce the frequency of edema caused by a plasma kallikrein dependent disorder, or of HAE attacks in a subject, and/or may reduce the severity and/or duration of an HAE attack in a subject, for example, the level and/or duration of edema experienced by the subject during an attack.

As used herein, “therapeutically effective inhibition of plasma kallikrein” refers to a level of inhibition of pKal in a subject that is effective to treat a plasma kallikrein dependent disorder, such as hereditary angioedema. For example, “therapeutically effective inhibition of plasma kallikrein” results from administration of a therapeutically effective amount of the anti-plasma kallikrein antibody disclosed herein.

As used herein, the term “threshold blood concentration (or level)” or “threshold circulating concentration (or level)” refers to a concentration of drug that must be achieved in the blood, i.e., a circulating concentration, in order for the drug, e.g., an anti-plasma kallikrein antibody to have a desired therapeutic effect, e.g., inhibition of plasma kallikrein at a level sufficient to treat a plasma kallikrein dependent disorder, e.g., to reduce the frequency and/or severity and/or duration of HAE attacks, or edema caused by a plasma kallikrein dependent disorder. As used herein “blood concentration (or level)” refers to circulating blood levels of the drug, e.g., the anti-plasma Kallikrein antibody. In some embodiments, the blood concentration or level (including threshold blood concentration or level) is measured as a serum concentration. In other embodiments, the blood concentration or level (including threshold blood concentration) is measured as a plasma concentration.

As used herein, the terms “treat” or “treatment” includes inhibition, prevention, or postponement or prevention of the development of the symptoms associated with a disorder and/or a reduction in the severity and/or duration of the symptoms of such disorder, such as plasma kallikrein dependent disorder, e.g., HAE. The terms further include ameliorating existing uncontrolled or unwanted symptoms, preventing additional symptoms, and ameliorating or preventing the underlying causes of such symptoms, e.g., plasma kallikrein activation that causes symptoms of a plasma kallikrein dependent disorder, e.g., HAE. For example, “treating” a plasma kallikrein dependent disorder, e.g., HAE, may include inhibiting, preventing or reducing the frequency of attacks and/or reducing the severity of attacks, and/or achieving a therapeutically effective inhibition of plasma kallikrein in a subject. In the context of HAE, treatment includes prophylactic measures taken to prevent HAE attacks, reduce the frequency and/or severity and/or duration of HAE attacks in a subject.

As used herein, the term “rescue medication” in the context of an HAE attack refers to an acute treatment administered to a subject experiencing symptoms of an HAE attack to treat the attack. For example, a rescue medication may include a C1 inhibitor or C1 esterase inhibitor. A rescue medication may also be referred to as “on-demand” treatment.

### 2. Anti-Plasma Kallikrein Antibodies

Anti-plasma kallikrein antibodies useful in treating plasma kallikrein dependent disorders, such as hereditary angioedema (HAE), according to the invention potently and specifically inhibit pKal activity and have long serum half-lives. For example, these antibodies have features useful in treating such disorders including specifically inhibiting pKal activity (e.g., K_{D} of 1.0 nM), having a lower affinity for prekallikrein (e.g., >1,000-fold lower affinity), and exhibiting no measurable inhibitory activity against other serine proteases. Anti-plasma kallikrein antibodies useful according to the invention contain a YTE-modified Fc domain (corresponding to M252Y, S254T, and T256E according to EU numbering) which enhances pH-dependent FcRn binding, resulting in a prolonged serum half-life (e.g., two to three months or longer). Antibodies useful in treating plasma kallikrein dependent disorders, including HAE, are described in International Patent Application Publication No. WO 2022/165130, the contents of which are incorporated by reference herein for all purposes.

Anti-plasma kallikrein antibodies useful in treating plasma kallikrein dependent disorders, e.g., HAE, according to the invention may be defined by their CDR sequences which are disclosed in Table 1 and Table 2 below using Kabat, Chothia, IMGT and ABM numbering schemes. It is contemplated that the CDR sequences can be identified using other numbering schemes known in the art (see, e.g., Dondelinger et al. (October 2018) Frontiers Immunol. 8, 2278, 1-15).

TABLE 1
Heavy Chain CDRs

| | HCDR1 | HCDR2 | HCDR3 |
|---|---|---|---|
| Kabat | SYWMN | QIYPGDDDTNYNAKFQG | SLMVTTGAPFDY |
| | (SEQ ID NO: 1) | (SEQ ID NO: 2) | (SEQ ID NO: 3) |
| Chothia | GYAFSSY | YPGDDD | SLMVTTGAPFDY |
| | (SEQ ID NO: 11) | (SEQ ID NO: 12) | (SEQ ID NO: 13) |
| IMGT | GYAFSSYW | IYPGDDDT | AGSLMVTTGAPFDY |
| | (SEQ ID NO: 17) | (SEQ ID NO: 18) | (SEQ ID NO: 19) |
| ABM | GYAFSSYWMN | QIYPGDDDTN | SLMVTTGAPFDY |
| | (SEQ ID NO: 22) | (SEQ ID NO: 23) | (SEQ ID NO: 13) |

TABLE 2
Light Chain CDRs

| | LCDR1 | LCDR2 | LCDR3 |
|---|---|---|---|
| Kabat | KASQDVGIAVA | YASHRGW | QQYRSYPLT |
| | (SEQ ID NO: 4) | (SEQ ID NO: 5) | (SEQ ID NO: 6) |
| Chothia | SQDVGIA | YAS | YRSYPLT |
| | (SEQ ID NO: 14) | (SEQ ID NO: 15) | (SEQ ID NO: 16) |
| IMGT | QDVGIA | YAS | QQYRSYPLT |
| | (SEQ ID NO: 20) | (SEQ ID NO: 15) | (SEQ ID NO: 21) |
| ABM | KASQDVGIAVA | YASHRGW | QQYRSYPLT |
| | (SEQ ID NO: 4) | (SEQ ID NO: 5) | (SEQ ID NO: 21) |

In one embodiment, the anti-plasma kallikrein antibody has a heavy chain (or heavy chain variable region) with a CDRH1 comprising the amino acid sequence of SEQ ID NO: 1, 11, 17, or 22; a CDRH2 comprising the amino acid sequence of SEQ ID NO:2, 12, 18, or 23; and a CDRH3 comprising the amino acid sequence of SEQ ID NO:3, 13, or 19.

In one embodiment, the anti-plasma kallikrein antibody has a light chain (or light chain variable region) with a CDRL1 comprising the amino acid sequence of SEQ ID NO:4, 14, or 20; a CDRL2 comprising the amino acid sequence of SEQ ID NO:5 or 15; and a CDRL3 comprising the amino acid sequence of SEQ ID NO:6, 16, or 21.

In another embodiment, the anti-plasma kallikrein antibody has a heavy chain (or heavy chain variable region) with a CDRH1 comprising the amino acid sequence of SEQ ID NO: 1, 11, 17, or 22; a CDRH2 comprising the amino acid sequence of SEQ ID NO:2, 12, 18, or 23; and a CDRH3 comprising the amino acid sequence of SEQ ID NO:3, 13, or 19; and a light chain (or light chain variable region) with a CDRL1 comprising the amino acid sequence of SEQ ID NO:4, 14, or 20; a CDRL2 comprising the amino acid sequence of SEQ ID NO: 5 or 15; and a CDRL3 comprising the amino acid sequence of SEQ ID NO:6, 16, or 21.

In another embodiment, the anti-plasma kallikrein antibody has a heavy chain (or heavy chain variable region) with a CDRH1 comprising the amino acid sequence of SEQ ID NO: 1; a CDRH2 comprising the amino acid sequence of SEQ ID NO:2; and a CDRH3 comprising the amino acid sequence of SEQ ID NO:3; and a light chain (or light chain variable region) with a CDRL1 comprising the amino acid sequence of SEQ ID NO:4; a CDRL2 comprising the amino acid sequence of SEQ ID NO:5; and a CDRL3 comprising the amino acid sequence of SEQ ID NO6.

The CDRs of the anti-plasma kallikrein antibody may be defined according to the Kabat definition, in which case the antibody comprises the heavy chain CDRs and light chain CDRs defined by Kabat in Tables 1 and 2 above. The CDRs of the anti-plasma kallikrein antibody may be defined according to the Chothia definition, in which case the antibody comprises the heavy chain CDRs and light chain CDRs defined by Chothia in Tables 1 and 2 above. The CDRs of the anti-plasma kallikrein antibody may be defined according to the IMGT definition, in which case the antibody comprises the heavy chain CDRs and light chain CDRs defined by IMGT in Tables 1 and 2 above. The CDRs of the anti-plasma kallikrein antibody may be defined according to the ABM definition, in which case the antibody comprises the heavy chain CDRs and light chain CDRs defined by ABM in Tables 1 and 2 above.

Antibodies defined by their CDRs according to the aforementioned embodiments may contain in their heavy chains an IgG1 constant region comprising an M252Y, an S254T, and a T256E mutation as numbered according to the EU numbering index. This series of mutations is also referred to as the “YTE” mutation, and is believed to enhance pH-dependent FcRn binding, resulting in a prolonged serum half-life of the antibody.

For example, in one embodiment, the anti-plasma kallikrein antibody useful according to the invention for the treatment of plasma kallikrein dependent disorders, e.g., HAE, has a heavy chain with a CDRH1 comprising the amino acid sequence of SEQ ID NO: 1; a CDRH2 comprising the amino acid sequence of SEQ ID NO:2; and a CDRH3 comprising the amino acid sequence of SEQ ID NO:3; and a light chain with a CDRL1 comprising the amino acid sequence of SEQ ID NO:4; a CDRL2 comprising the amino acid sequence of SEQ ID NO:5; and a CDRL3 comprising the amino acid sequence of SEQ ID NO: 6. The heavy chain also has an IgG1 constant region with mutations M252Y, S254T, and T256E mutation as numbered according to the EU numbering index.

In some embodiments, the anti-plasma kallikrein antibody is defined by its heavy and/or light chain variable region sequences which are shown in Table 3 below.

TABLE 3
Variable Region Sequences

| | |
|---|---|
| Heavy Chain Variable | QVQLVQSGAEVKKPGSSVKVSCKASGYAFSSYWMNWVRQAPGQGL |
| Region | EWIGQIYPGDDDTNYNAKFQGRVTITVDKSTTTAYMELSSLRSED |
| | TAVYFCAGSLMVTTGAPFDYWGQGTTVTVSS (SEQ ID NO: 7) |
| Light Chain Variable | DIQMTQSPSSLSASVGDRVTITCKASQDVGIAVAWYQQKPGKAPK |
| Region | FLIYYASHRGWGVPDRFSGSGSGTDFTLTISSLQPEDFATYFCQQ |
| | YRSYPLTFGQGTKLEIK (SEQ ID NO: 8) |

The antibody may comprise the Complementarity Determining Sequences (CDRs) of a heavy chain variable region having the amino acid sequence of SEQ ID NO: 7, and CDRs of a light chain variable region having the amino acid sequence of SEQ ID NO: 8. Further, the heavy chain of the antibody has an IgG1 constant region with mutations M252Y, S254T, and T256E mutation as numbered according to the EU numbering index.

In one embodiment, the anti-plasma kallikrein antibody has a heavy chain variable region sequence having the amino acid sequence of SEQ ID NO:7. In another embodiment, the anti-plasma kallikrein antibody has a light chain variable region sequence having the amino acid sequence of SEQ ID NO:8.

For example, in one embodiment, the anti-plasma kallikrein antibody has the a heavy chain variable region having the amino acid sequence of SEQ ID NO:7 and a light chain variable region having the amino acid sequence of SEQ ID NO:8.

In another embodiment, the anti-plasma kallikrein antibody has a heavy chain variable region having the amino acid sequence of SEQ ID NO:7 and a light chain variable region having the amino acid sequence of SEQ ID NO:8, and the heavy chain has an IgG1 constant region with the mutations M252Y, S254T, and T256E as numbered according to the EU numbering index.

In some embodiments, the anti-plasma kallikrein antibody is defined by its full length heavy and/or light chain sequences which are shown in Table 4 below.

TABLE 4
Full Length Sequences

| | |
|---|---|
| Heavy Chain | QVQLVQSGAEVKKPGSSVKVSCKASGYAFSSYWMNWVRQAPGQGLEWIGQIY |
| | PGDDDTNYNAKFQGRVTITVDKSTTTAYMELSSLRSEDTAVYFCAGSLMVTT |
| | GAPFDYWGQGTTVTVSSASTKGPSVFPLAPSSKSTSGGTAALGCLVKDYFPE |
| | PVTVSWNSGALTSGVHTFPAVLQSSGLYSLSSVVTVPSSSLGTQTYICNVNH |
| | KPSNTKVDKRVEPKSCDKTHTCPPCPAPELLGGPSVFLFPPKPKDTLYITRE |
| | PEVTCVVVDVSHEDPEVKFNWYVDGVEVHNAKTKPREEQYNSTYRVVSVLTV |
| | LHQDWLNGKEYKCKVSNKALPAPIEKTISKAKGQPREPQVYTLPPSREEMTK |
| | NQVSLTCLVKGFYPSDIAVEWESNGQPENNYKTTPPVLDSDGSFFLYSKLTV |
| | DKSRWQQGNVFSCSVMHEALHNHYTQKSLSLSPG |
| | (SEQ ID NO: 9) |
| Light Chain | DIQMTQSPSSLSASVGDRVTITCKASQDVGIAVAWYQQKPGKAPKFLIYYAS |
| | HRGWGVPDRFSGSGSGTDFTLTISSLQPEDFATYFCQQYRSYPLTFGQGTKL |
| | EIKRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKS |
| | FNRGEC |
| | (SEQ ID NO: 10) |

For example, in one embodiment, the anti-plasma kallikrein antibody has a heavy chain having the amino acid sequence of SEQ ID NO:9, which includes an IgG1 constant region with mutations M252Y, S254T, and T256E as numbered according to the EU numbering index; the mutated residues are underlined in Table 4 above. In another embodiment, the anti-plasma kallikrein antibody has a light chain having the amino acid sequence of SEQ ID NO: 10.

In a further embodiment, the anti-plasma kallikrein antibody has a heavy chain having the amino acid sequence of SEQ ID NO:9 and a light chain having the amino acid sequence of SEQ ID NO: 10, and the heavy chain sequence has an IgG1 constant region with mutations M252Y, S254T, and T256E mutation as numbered according to the EU numbering index; the mutated residues are underlined in Table 4 above. For example, the antibody is STAR-0215.

### 3. Dosing & Administration

The invention provides methods of treating plasma kallikrein dependent disorders, e.g., hereditary angioedema (HAE), in a subject diagnosed with and/or suffering from such a disorder by administering an anti-plasma kallikrein antibody as disclosed herein. In particular, the methods disclosed herein treat plasma kallikrein dependent disorders, e.g., HAE, through infrequent dosing of the anti-plasma kallikrein antibodies disclosed herein, e.g., about every month, about every two months, about every three months, about every four months, about every five months, about every six months, about every nine months, or about every twelve months or more. Reducing the frequency of dosing, as compared to other available treatments for HAE, improves, e.g., patient compliance and quality of life, leading to better disease management.

The methods of administering the anti-plasma kallikrein antibodies disclosed herein achieve, maintain or exceed a desired threshold blood concentration, e.g., a serum or plasma concentration of the anti-plasma kallikrein antibody to ensure therapeutically effective levels of pKal inhibition for a prolonged period of time, e.g., about 2 months, about 3 months, about 4 months, about 5 months, about 6 months, or even longer, such as about 9 months or about 12 months.

Accordingly, disclosed herein are dosing regimens that maintain the blood concentration (e.g., as measured in plasma or serum) of the antibody above the threshold blood concentration required for therapeutically effective levels of pKal inhibition. Dosing regimens disclosed herein may involve the following general strategies:
(i) repeated administration of a fixed dose (each dose is the same) at equal or varying time intervals;
(ii) administration of doses, e.g., fixed doses, in repeated “treatment cycles” where the cycles include administration of specific doses at specific time intervals.
(iii) administration of one or more loading doses (e.g., one or more initial higher doses) followed by maintenance doses (e.g., lower doses) administered at equal or varying time intervals;

A prolonged duration of action may be achieved by administration of a single dose of the anti-plasma kallikrein antibody. For example, a single dose of the anti-plasma kallikrein antibody may be sufficient to maintain the required blood concentration for therapeutically effective levels of pKal inhibition for a period of time, e.g., about 2 months, about 3 months, about 4 months, about 5 months, about 6 months, or even longer, such as about 9 months or about 12 months. The duration of action of a single dose can be extended by repeated dosing of the antibody according to the methods disclosed herein.

The antibodies disclosed herein can be administered periodically, e.g., about every 2 months, every 3 months, every 4 months, every 5 months, every 6 months, every 9 months, or every 12 months, or more, to maintain the blood concentration (e.g., as measured in plasma or serum) of the antibody above the threshold blood concentration required for therapeutically effective levels of pKal inhibition. The doses administered periodically may be fixed doses of the same amount, or they may be of different amounts, e.g., a first dose that is a loading dose, followed by subsequent lower maintenance doses administered periodically. For example, administration of a single dose of the anti-plasma kallikrein antibody may be sufficient to achieve, maintain or exceed a threshold blood concentration (e.g., as measured in serum or plasma) of the antibody to provide therapeutically effective levels of pKal inhibition for, e.g., three months, thereby treating the plasma kallikrein dependent disorders, e.g., HAE, in a subject for a period of time, e.g., about 2 months, about 3 months, about 4 months, about 5 months, about six months, or even longer, such as for about 9 months, or even 12 months. As the antibody is eliminated from circulation over time, the subject is administered another dose of the antibody to provide a blood concentration of the antibody that again exceeds the threshold blood concentration for at least two months, at least three months or more. This “another dose” may be the same amount as the first dose, or it may be a different dose, such a lower “maintenance dose.” Dosing of the antibody can continue periodically every two months, every three months, every four months, every five months, every six months or more, such as every nine months or every twelve months, and can be administered chronically (e.g., for years) to treat the disorder over the lifetime of the patient. In some embodiments, dosing is every three months. In other embodiments, dosing is every six months.

In one embodiment, the anti-pKal antibody is administered according to the methods disclosed herein to achieve in a subject a blood concentration (e.g., as measured in plasma or serum) concentration of the antibody at or exceeding about 1 μg/mL, 2 μg/mL, 3 μg/mL, 4 μg/mL, 5 μg/mL, 6 μg/mL, 7 μg/mL, 8 μg/mL, 9 g/mL, 10 μg/mL, 11 μg/mL, 12 μg/mL, 13 μg/mL, 14 μg/mL, 15 μg/mL, 16 μg/mL, 17 μg/mL, 18 μg/mL, 19 μg/mL, 20 μg/mL, 21 μg/mL, 22 μg/mL, 23 μg/mL, 24 μg/mL, 25 μg/mL, 26 μg/mL, 27 μg/mL, 28 μg/mL, 29 μg/mL, 30 μg/mL, 31 μg/mL, 32 μg/mL, 33 μg/mL, 34 μg/mL, 35 μg/mL, 36 μg/mL, 37 μg/mL, 38 μg/mL, 39 μg/mL, 40 μg/mL, 41 μg/mL, 42 μg/mL, 43 μg/mL, 44 μg/mL, 45 μg/mL, 46 μg/mL, 47 μg/mL, 48 μg/mL, 49 μg/mL, 50 μg/mL, 51 μg/mL, 52 μg/mL, 53 μg/mL, 54 μg/mL, 55 μg/mL, 56 μg/mL, 57 μg/mL, 58 μg/mL, 59 μg/mL, 60 μg/mL, 61 μg/mL, 62 μg/mL, 63 μg/mL, 64 μg/mL, 65 μg/mL, 66 μg/mL, 67 μg/mL, 68 μg/mL, 69 μg/mL, 70 μg/mL, 71 μg/mL, 72 μg/mL, 73 μg/mL, 74 μg/mL, 75 μg/mL, 76 μg/mL, 77 μg/mL, 78 μg/mL, 79 μg/mL, 80 μg/mL, 81 μg/mL, 82 μg/mL, 83 μg/mL, 84 μg/mL, 85 μg/mL, 86 μg/mL, 87 μg/mL, 88 μg/mL, 89 μg/mL, 90 μg/mL, 91 μg/mL, 92 μg/mL, 93 μg/mL, 94 μg/mL, 95 μg/mL, 96 μg/mL, 97 μg/mL, 98 μg/mL, 99 μg/mL, or 100 μg/mL. In one particular embodiment, the blood, e.g., serum concentration of the anti-plasma kallikrein antibody exceeds about 8 μg/mL, or about 10 μg/mL. For example, the antibody is administered to exceed a blood concentration, e.g., serum or plasma concentration of the antibody, of about 12 μg/mL. For example, the antibody is administered to provide a blood, e.g., serum concentration of the antibody of greater than or equal to 25 μg/mL.

In one embodiment, the anti-plasma kallikrein antibody is administered according to the methods disclosed herein to achieve or maintain a blood concentration (e.g., as measured in plasma or serum) in the subject between 8-20 μg/mL, 10-20 μg/mL, 12-20 μg/mL, 8-30 μg/mL, 10-30 μg/mL, 12-30 μg/mL, 8-40 μg/mL, 10-40 μg/mL, 12-40 μg/mL, 8-50 g/mL, 10-50 μg/mL, 12-50 μg/mL, 8-60 μg/mL, 10-60 μg/mL, 12-60 μg/mL, 8-70 μg/mL, 10-70 μg/mL, 12-70 μg/mL, 8-80 μg/mL, 10-80 μg/mL, 12-80 μg/mL, 8-90 μg/mL, 10-90 μg/mL, 12-90 μg/mL, 8-100 μg/mL, 10-100 μg/mL, or 12-100 μg/mL. The blood concentration may be maintained or exceeded for the stated period of time by administration of a single dose of the antibody. The single dose may be a loading dose or maintenance dose as disclosed herein. For example, the anti-plasma kallikrein antibody is administered as a single dose to achieve a blood concentration (e.g., as measured in plasma or serum) of about 8-60 μg/mL, 10-60 μg/mL, or about 12-60 μg/mL for at least about two months, about three months, about four months, about five months, about six months, about 9 months, or about 12 months. In one embodiment, the blood concentration is maintained for about three months. In one embodiment, the blood concentration is maintained for about six months. In one embodiment, the blood concentration is maintained for about nine months. In one embodiment, the blood concentration is maintained for about twelve months.

In one embodiment, the threshold blood concentration (e.g., as measured in plasma or serum) concentration of the anti-plasma kallikrein antibody administered according to the methods disclosed herein is in a range of about 60-80 nM. For example, the threshold blood concentration is in the range of about 70-80 nM, or about 75-80 nM. For example, the threshold blood concentration is in the range of about 150-170 nM. In one embodiment, the blood concentration, e.g., the serum or plasma concentration, of the anti-plasma kallikrein antibody exceeds about 60 nM, about 61 nM, about 62 nM, about 63 nM, about 64 nM, about 65 nM, about 66 nM, about 67 nM, about 68 nM, about 69 nM, about 70 nM, about 71 nM, about 72 nM, about 73 nM, about 74 nM, about 75 nM, about 76 nM, about 77 nM, about 78 nM, about 79 nM, or about 80 nM. In another embodiment, the blood concentration, e.g., the serum or plasma concentration, of the anti-plasma kallikrein antibody exceeds about 100 nM, or about 110 nM, or about 120 nM, or about 130 nM, or about 140 nM, or about 150 nM, or about 160 nM. For example, in one embodiment, the serum concentration of the antibody is about 167 nM.

The threshold blood concentration (e.g., as measured in plasma or serum) of the anti-plasma kallikrein antibody may be maintained or exceeded for about one month, about two months, about three months, about four months, about five months, or about six months or more. The threshold blood concentration may be maintained or exceeded for the stated period of time by administration of a single dose of the antibody. The single dose may be a loading dose or maintenance dose as disclosed herein. In one particular embodiment, the threshold blood concentration is maintained or exceeded for about three months. In another embodiment, the blood concentration, e.g. the serum or plasma concentration, of the anti-plasma kallikrein antibody is maintained at or exceeds about 8 μg/mL or about 10 μg/mL, e.g., about 12 μg/mL for about three months. In one embodiment, the anti-plasma kallikrein antibody is administered in a single dose in an amount to achieve a blood concentration, e.g., a serum or plasma concentration, of the antibody that meets or exceeds, for example, about 10 μg/mL, e.g., about 12 μg/mL for about three months or about 12 weeks or about 84 days or about 90 days.

The threshold blood concentration (e.g., as measured in plasma or serum) may be maintained or exceeded for 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 91 days, 92 days, 93 days, 94 days, 95 days, 96 days, 97 days, 98 days, 99 days, 100 days, 101 days, 102 days, 103 days, 104 days, 105 days, 106 days, 107 days, 108 days, 109 days, 110 days, 111 days, 112 days, 113 days, 114 days, 115 days, 116 days, 117 days, 118 days, 119 days, or 120 days. The threshold blood concentration may be maintained or exceeded for about 1 month, about 2 months, about 3 months, about 4 months, about 5 months, about 6 months, about 7 months, about 8 months, about 9 months, about 10 months, about 11 months, or about 12 months. The threshold blood concentration may be maintained or exceeded for 3 months. The threshold blood concentration may be maintained or exceeded for 6 months. The threshold blood concentration may be maintained or exceeded for the stated period of time by administration of a single dose of the antibody. The single dose may be a loading dose or maintenance dose as disclosed herein. The threshold blood concentration may be maintained at or exceed about 12-100 μg/mL, e.g., about 12 μg/mL, for the stated period by administration of one dose of the anti-plasma kallikrein antibody. The threshold blood concentration may be maintained at or above about 12 μg/mL by administration of a loading dose and subsequent maintenance doses according to the disclosure. The threshold blood concentration may be maintained at or above about 12 μg/mL by administering fixed doses at fixed or varying intervals according to the disclosure. The threshold blood concentration may be maintained at or above about 12 μg/mL by administering treatment cycles according to the disclosure.

The threshold blood concentration may be maintained or exceeded for about 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, or more. The threshold blood concentration may be maintained or exceeded for the stated period of time by administration of a single dose of the antibody. The single dose may be a loading dose or maintenance dose as disclosed herein. The threshold blood concentration, e.g., the serum or plasma concentration, maintained or exceeded may be about 8 μg/mL, or about 10 μg/mL, or about 12 μg/mL, for the stated period of time.

Disclosed is a method for maintaining a blood (e.g., serum or plasma) concentration of greater than about 12 μg/mL of an anti-plasma kallikrein antibody, e.g., STAR-0215, in subject with hereditary angioedema (HAE). The method comprises administering to the subject an anti-plasma kallikrein antibody comprising two heavy chains and two light chains, wherein each heavy chain comprises the amino acid sequence of SEQ ID NO: 9 and each light chain comprises the amino acid sequence of SEQ ID NO:10. According to the method, the antibody is administered according to one of the following dosing regimens:
(1) administering a loading dose of 600 mg followed by administering a maintenance dose of 300 mg three months after the loading dose;
(2) administering a first dose of 600 mg followed by administering a second dose of 600 mg about 28 days after the first dose; or
(3) administering at least 450 mg of the antibody to the subject every six months;
wherein the serum concentration exceeds 12 μg/mL for at least three months from a loading dose or first dose of the antibodies. The blood concentration may be exceeded for at least three months, e.g., at least six months, according to the method. When the dosing regimen is (1) above, the blood concentration of 12 μg/mL is exceeded for at least 3 months from administering the loading dose, or from administering the maintenance dose. When the dosing regimen is (2) above, the blood concentration of 12 μg/mL is exceeded for at least 6 months from administration of the first dose, or for at least six months from administration of the second dose. When the dosing regimen is (3) above, the blood concentration of 12 μg/mL is exceeded for at least 6 months from administration of the dose.

According to the methods disclosed herein, the anti-plasma kallikrein antibody is administered as a single dose of about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1,000 mg, about 1,025 mg, about 1,050 mg, about 1,075 mg, about 1,100 mg, about 1,125 mg, about 1,150 mg, about 1,175 mg, about 1,200 mg, about 1,225 mg, about 1,250 mg, about 1,275 mg, about 1,300 mg, about 1,325 mg, about 1,350 mg, about 1,375 mg, about 1,400 mg, about 1,425 mg, about 1,450 mg, about 1,475 mg, about 1,500 mg, about 1,525 mg, about 1,550 mg, about 1,575 mg, about 1,600 mg, about 1,625 mg, about 1,650 mg, about 1,675 mg, about 1,700 mg, about 1,725 mg, about 1,750 mg, about 1,775 mg, about 1,800 mg, about 1,825 mg, about 1,850 mg, about 1,875 mg, about 1,900 mg, about 1,925 mg, about 1,950 mg, about 1,975 mg, or about 2,000 mg of the antibody to achieve a therapeutically effective level of inhibition of plasma kallikrein in the subject. The single dose may be administered periodically at regular intervals, e.g., every two months, every three months, every four months, every five months, every six months, every seven months, every eight months, every nine months, or every 12 months. The single dose may be a loading dose or maintenance dose as disclosed herein. The dose is a weight-independent dose; in other words, the dose is an absolute dose or fixed dose that is independent of the patient's weight. The dose may be administered chronically and periodically at least every two months, three months, four months, five months, six months or more, with every three months being a preferred period for certain doses, while six months, 9 months or 12 months may be preferred for other doses. The dose may be a fixed dose for the duration of treatment (e.g., the first and subsequent doses are the same). For example, in one embodiment, the dose is 450 mg administered, e.g., every three months, e.g., by subcutaneous administration. In another embodiment, the dose is 1,200 mg administered, e.g., every six months, e.g., by subcutaneous administration. In another embodiment, the dose is 600 mg administered, e.g., every six months, e.g., by intravenous administration. In another embodiment, the dose is 600 mg administered, e.g., every six months, e.g., by subcutaneous administration. In another embodiment, the dose is 1,200 mg administered, e.g., every twelve months, e.g., by intravenous administration.

According to the methods disclosed herein, the anti-plasma kallikrein antibody is administered as a single dose of about 100-600 mg, about 100-700 mg, about 100-800 mg, about 100-900 mg, about 100-1,000 mg, about 100-1,100 mg, about 100-1,200 mg, about 100-1,300 mg, about 100-1,400 mg, about 100-1,500 mg, about 100-1,600 mg, about 100-1,700 mg, about 100-1,800 mg, about 100-1,900 mg, about 100-2,000 mg, about 200-600 mg, about 200-700 mg, about 200-800 mg, about 200-900 mg, about 200-1,000 mg, about 200-1,100 mg, about 200-1,200 mg, about 200-1,300 mg, about 200-1,400 mg, about 200-1,500 mg, about 200-1,600 mg, about 200-1,700 mg, about 200-1,800 mg, about 200-1,900 mg, about 200-2,000 mg, about 300-600 mg, about 300-700 mg, about 300-800 mg, about 300-900 mg, about 300-1,000 mg, about 300-1,100 mg, about 300-1,200 mg, about 300-1,300 mg, about 300-1,400 mg, about 300-1,500 mg, about 300-1,600 mg, about 300-1,700 mg, about 300-1,800 mg, about 300-1,900 mg, about 300-2,000 mg, about 400-600 mg, about 400-700 mg, about 400-800 mg, about 400-900 mg, about 400-1,000 mg, about 400-1,100 mg, about 400-1,200 mg, about 400-1,300 mg, about 400-1,400 mg, about 400-1,500 mg, about 400-1,600 mg, about 400-1,700 mg, about 400-1,800 mg, about 400-1,900 mg, about 400-2,000 mg, about 500-600 mg, about 500-700 mg, about 500-800 mg, about 500-900 mg, about 500-1,000 mg, about 500-1,100 mg, about 500-1,200 mg, about 500-1,300 mg, about 500-1,400 mg, about 500-1,500 mg, about 500-1,600 mg, about 500-1,700 mg, about 500-1,800 mg, about 500-1,900 mg, about 500-2,000 mg, about 600-700 mg, about 600-800 mg, about 600-900 mg, about 600-1,000 mg, about 600-1,100 mg, about 600-1,200 mg, about 600-1,300 mg, about 600-1,400 mg, about 600-1,500 mg, about 600-1,600 mg, about 600-1,700 mg, about 600-1,800 mg, about 600-1,900 mg, about 600-2,000 mg, about 700-800 mg, about 700-900 mg, about 700-1,000 mg, about 700-1,100 mg, about 700-1,200 mg, about 700-1,300 mg, about 700-1,400 mg, about 700-1,500 mg, about 700-1,600 mg, about 700-1,700 mg, about 700-1,800 mg, about 700-1,900 mg, about 700-2,000 mg, about 800-900 mg, about 800-1,000 mg, about 800-1,100 mg, about 800-1,200 mg, about 800-1,300 mg, about 800-1,400 mg, about 800-1,500 mg, about 800-1,600 mg, about 800-1,700 mg, about 800-1,800 mg, about 800-1,900 mg, about 800-2,000 mg, about 900-1,000 mg, about 900-1,100 mg, about 900-1,200 mg, about 900-1,300 mg, about 900-1,400 mg, about 900-1,500 mg, about 900-1,600 mg, about 900-1,700 mg, about 900-1,800 mg, about 900-1,900 mg, about 900-2,000 mg, about 1,000-1,100 mg, about 1,000-1,200 mg, about 1,000-1,300 mg, about 1,000-1,400 mg, about 1,000-1,500 mg, about 1,000-1,600 mg, about 1,000-1,700 mg, about 1,000-1,800 mg, about 1,000-1,900 mg, about 1,000-2,000 mg, about 1,100-1,200 mg, about 1,100-1,300 mg, about 1,100-1,400 mg, about 1,100-1,500 mg, about 1,100-1,600 mg, about 1,100-1,700 mg, about 1,100-1,800 mg, about 1,100-1,900 mg, about 1,100-2,000 mg, about 1,200-1,300 mg, about 1,200-1,400 mg, about 1,200-1,500 mg, about 1,200-1,600 mg, about 1,200-1,700 mg, about 1,200-1,800 mg, about 1,200-1,900 mg, about 1,200-2,000 mg, about 1,300-1,400 mg, about 1,300-1,500 mg, about 1,300-1,600 mg, about 1,300-1,700 mg, about 1,300-1,800 mg, about 1,300-1,900 mg, about 1,300-2,000 mg, about 1,400-1,500 mg, about 1,400-1,600 mg, about 1,400-1,700 mg, about 1,400-1,800 mg, about 1,400-1,900 mg, about 1,400-2,000 mg, about 1,500-1,600 mg, about 1,500-1,700 mg, about 1,500-1,800 mg, about 1,500-1,900 mg, about 1,500-2,000 mg, about 1,600-1,700 mg, about 1,600-1,800 mg, about 1,600-1,900 mg, about 1,600-2,000 mg, about 1,700-1,800 mg, about 1,700-1,900 mg, about 1,700-2,000 mg, about 1,800-1,900 mg, about 1,800-2,000 mg, or about 1,900-2,000 mg of the antibody to achieve a therapeutically effective level of inhibition of plasma kallikrein in the subject. The single dose may be a loading dose or maintenance dose as disclosed herein. The dose is a weight-independent dose; in other words, the dose is an absolute dose or fixed dose that is independent of the patient's weight. The dose may be administered chronically and periodically at least every two months, three months, four months, five months, six months or more, with every three months being a preferred period for certain doses, while six months, 9 months or 12 months may be preferred for other doses. The dose may be a fixed dose for the duration of treatment (e.g., the first and subsequent doses are the same). For example, the dose may be 1,200 mg every 6 months. For example, the dose may be 1,100-1,500 mg every 6 months. For example, the dose may be 1,100-1,500 mg every 12 months. For example, the dose may be 1,200 mg every 12 months.

The dose may be administered, for example about every month, every two months, every three months, every four months, every five months, or every six months or more, such as every nine months or every 12 months. The dose may be administered at intervals to maintain the therapeutically effective level of inhibition of plasma kallikrein in the subject, for example about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, about every 16 weeks, about every 17 weeks, about every 18 weeks, about every 19 weeks, about every 20 weeks, about every 21 weeks, about every 22 weeks, about every 23 weeks, about every 24 weeks, about every 25 weeks, about every 26 weeks. The dose may be a fixed dose for the duration of treatment (e.g., the first and subsequent doses are the same), or the dose may be a maintenance dose (e.g., after a loading dose).

For example, the dose may be administered about every 28 days, about every 30 days about every 35 days, about every 42 days, about every 49 days, about every 56 days, about every 60 days, about every 63 days, about every 70 days, about every 77 days, about every 84 days, about every 90 days, about every 91 days, about every 98 days, about every 105 days, about every 112 days, about every 119 days, about every 120 days about every 126 days, about every 133 days, about every 140 days, about every 147 days, about every 150 days, about every 154 days, about every 161 days, about every 168 days, about every 175 days, about every 180 days, or about every 182 days. For example, the dose is administered about every three months, or about every twelve weeks or about every 90 days.

According to the methods disclosed herein, the anti-plasma kallikrein antibody is administered as a single dose of about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1,000 mg, about 1,025 mg, about 1,050 mg, about 1,075 mg, about 1,100 mg, about 1,125 mg, about 1,150 mg, about 1,175 mg, about 1,200 mg, about 1,225 mg, about 1,250 mg, about 1,275 mg, about 1,300 mg, about 1,325 mg, about 1,350 mg, about 1,375 mg, about 1,400 mg, about 1,425 mg, about 1,450 mg, about 1,475 mg, about 1,500 mg, about 1,525 mg, about 1,550 mg, about 1,575 mg, about 1,600 mg, about 1,625 mg, about 1,650 mg, about 1,675 mg, about 1,700 mg, about 1,725 mg, about 1,750 mg, about 1,775 mg, about 1,800 mg, about 1,825 mg, about 1,850 mg, about 1,875 mg, about 1,900 mg, about 1,925 mg, about 1,950 mg, about 1,975 mg, or about 2,000 mg of the antibody to achieve a therapeutically effective level of inhibition of plasma kallikrein in the subject. The dose may be a fixed dose for the duration of treatment (e.g., the first and subsequent doses are the same). The single dose may be a loading dose or maintenance dose as disclosed herein. The dose is a weight-independent dose; in other words, the dose is an absolute dose or fixed dose that is independent of the patient's weight. The dose may be administered at periodic intervals to maintain the therapeutically effective level of inhibition of plasma kallikrein in the subject, for example about every month, every two months, every three months, every four months, every five months, or every six months or more, with every three months being a preferred period for dosing.

### (i). Repeated Administration of Fixed Doses

Exemplary approaches involving repeated administration of fixed doses (each dose is the same) of anti-plasma kallikrein antibodies at equal or varying time intervals are described in the following sections.

### A. Fixed Doses Administered at Equal Time Intervals

In some embodiments of the methods of treating plasma kallikrein dependent disorders, e.g., hereditary angioedema (HAE) using anti-plasma kallikrein antibodies, a first dose, a second dose, and subsequent doses of the anti-plasma kallikrein antibody are administered at equal time intervals over the course of treatment. These doses are fixed doses (e.g., every dose is the same amount). In some embodiments, a first dose of the anti-plasma kallikrein antibody, e.g., STAR-0215, is administered to the subject, and after at first period of time from administering the first dose, a second dose of the anti-plasma kallikrein antibody is administered to the subject. Thereafter, at intervals equal to the first period of time one or more subsequent doses of the anti-plasma kallikrein antibody are administered to the subject. The first dose, second dose, and subsequent doses are the same.

For example, the first period of time is between about 3 months and about 9 months, e.g., about 3 months, about 4 months, about 5 months, about 6 months, about 7 months, about 8 months, or about 9 months. Accordingly, in one embodiment, when the first period of time is about three months, the subject receives a dose of the antibody about every three months. Alternatively, when the first period of time is about four months, the subject receives a dose of the antibody about every four months. Alternatively, when the first period of time is about five months, the subject receives a dose of the antibody about every five months. Alternatively, when the first period of time is about six months, the subject receives a dose of the antibody about every six months. Alternatively, when the first period of time is about seven months, the subject receives a dose of the antibody about every seven months. Alternatively, when the first period of time is about eight months, the subject receives a dose of the antibody about every eight months. Alternatively, when the first period of time is about nine months, the subject receives a dose of the antibody about every nine months. Alternatively, when the first period of time is about twelve months, the subject receives a dose of the antibody about every twelve months.

In some embodiments, the fixed dose, e.g., the first dose, second dose, and any subsequent dose are the same amount. For example, the dose is 300-2,000 mg. For example, the dose is 500-1,500 mg. For example, the dose is 500-550 mg, 550-600 mg, 600-650 mg, 650-700 mg, 700-750 mg, 750-800 mg, 850-900 mg, 900-50 mg, 950-1,000 mg, 1,000-1,050 mg, 1,050-1,100 mg, 1,100-1,150 mg, 1,150-1,200 mg, 1,200-1,250 mg, 1,250-1,300 mg, 1,300-1,350 mg, 1,350-1,400 mg, 1,400-1,450 mg, 1,450-1,500 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1,000 mg, 1,025 mg, 1,050 mg, 1,075 mg, 1,100 mg, 1,125 mg, 1,150 mg, 1,175 mg, 1,200 mg, 1,225 mg, 1,250 mg, 1,275 mg, 1,300 mg, 1,325 mg, 1,350 mg, 1,375 mg, 1,400 mg, 1,425 mg, 1,450 mg, 1,475 mg, or 1,500 mg. For example, the dose is 600-1,200 mg. For example, the dose is 1,100-1,500 mg. For example, the dose is 300-450 mg. For example, the dose is 300 mg. For example, the dose is 400 mg. For example, the dose is 450 mg. For example, the dose is 400-600 mg. For example, the dose is 600 mg. For example, the dose is 600-900 mg. For example, the dose is 700 mg. For example, the dose is 900 mg. For example, the dose is 1,200 mg. For example, in some embodiments, the dose is 1,200 mg every six months. In some embodiments, the dose 600 mg every three months. In some embodiments, the dose is 600 mg every six months. In some embodiments, the dose is 700 mg every six months. In some embodiments, the dose is 300-450 mg every three months. In some embodiments, the dose is 400-600 mg every three months. In some embodiments, the dose is 400-600 mg every six months. In some embodiments, the dose is 600-900 mg every six months. In some embodiments, the dose is 600 mg every six months. In some embodiments, the dose is 900 mg every six months. In some embodiments, the dose is 1,200 mg every six months. In other embodiments, the dose is 1,100-1,500 mg every twelve months. In other embodiments, the dose is 900-1,500 mg every twelve months. In some embodiments, the dose is 1,200 mg every twelve months.

In one embodiment, all doses are administered intravenously. In another embodiment, all doses are administered subcutaneously. In another embodiment, the first dose is administered intravenously and the second and subsequent doses are administered subcutaneously. For example, a first, second, and subsequent doses are 600-1,200 mg, e.g., 600 mg administered subcutaneously. For example, a first, second, and subsequent doses are 600-1,200 mg, e.g., 1,200 mg administered subcutaneously. For example, a first, second, and subsequent doses are 600-1,200 mg, e.g., 600 mg administered intravenously. For example, a first, second, and subsequent doses are 600-1,200 mg, e.g., a first dose of 1,200 mg administered intravenously, followed by second and subsequent doses of 1,200 mg administered subcutaneously. For example, a first, second, and subsequent doses are 600-1,200 mg, e.g., a first dose of 600 mg administered intravenously, followed by second and subsequent doses of 600 mg administered subcutaneously. For example, in one embodiment, a first dose of 600 mg is administered intravenously, followed by a second dose of 600 mg about 6 months later intravenously and about every six months thereafter. For, example, in one embodiment, a first dose of 1,200 mg is administered intravenously, followed by a second dose of 1,200 mg about 6 months later subcutaneously and about every six months thereafter. For example, in one embodiment, a first dose of 1,200 mg is administered subcutaneously, followed by a second dose of 1,200 mg about 6 months later subcutaneously and about every six months thereafter. For example, in one embodiment, a first dose of 600-1,200 mg is administered subcutaneously, followed by a second dose of 600-1,200 mg about 6 months later subcutaneously and about every six months thereafter, where all doses are the same. For example, in one embodiment, a first dose of 600-1,200 mg, e.g., 600 mg is administered intravenously, followed by a second dose of 600-1,200 mg, e.g., 600 mg about 6 months later intravenously and about every six months thereafter, where all doses are the same. For example, in one embodiment, a first dose of 600-1,200 mg is administered subcutaneously, followed by a second dose of 600-1,200 mg about 6 months later subcutaneously and about every six months thereafter, where all doses are the same.

### B. Fixed Doses Administered at Varying Time Intervals

Other embodiments using fixed doses (i.e., doses of the same amount) of an anti-plasma kallikrein antibody, such as STAR-0215, (e.g., every dose is the same) to treat plasma kallikrein dependent disorders, e.g., hereditary angioedema (HAE), involve administering dose(s) of the antibody initially at a first shorter intervals and then changing to a second longer interval between doses. In some embodiments, a first dose of the anti-plasma kallikrein antibody is administered to the subject, and after at first period of time from administering the first dose, a second dose of the anti-plasma kallikrein antibody is administered to the subject, and after a second period of time equal to the first period of time, a third dose of the anti-plasma kallikrein antibody is administered to the subject. Thereafter, at intervals equal to a third period of time one or more subsequent doses of the anti-plasma kallikrein antibody are administered to the subject. The third period of time is longer that the first and second periods of time. According to some embodiments, the first period of time may be between about 2 months and about 6 months, e.g., about 2 months, about 3 months, about 4 months, about 5 months, or about 6 months; and the third period of time may be between about 4 months and about 9 months, e.g., about 3 months, about 4 months, about 5 months, about 6 months, about 7 months, about 8 months, or about 9 months. Accordingly, in one embodiment, the first period of time is about two months, and the third period of time is about four months, such that the subject receives a dose of the antibody about every four months after the third dose. Alternatively, the first period of time is about three months, and the third period of time is about six months, such that the subject receives a dose of the antibody about every six months after the third dose. Alternatively, the first period of time is about four months and the third period of time is about eight months, such that the subject receives a dose of the antibody about every eight months after the third dose. Alternatively, the first period of time is about four months and the third period of time is about nine months, such that the subject receives a dose of the antibody about every eight months after the third dose. In some embodiments, the second dose may occur as early as 1 week after the first dose. For example, the first period of time may be 1-4 weeks, e.g., about 1 week, about 2 weeks, about 3 weeks, or about 4 weeks. For example, the first period of time may be about 1 week. In some embodiments, the first period of time may be about 1 week, about 2 weeks, about 3 weeks, or about 4 weeks, e.g., about 1 week, and the third period of time is about 3 months, about 4 months, about 5 months, or about 6 months, e.g., about 3 months. In some embodiments, the first period of time may be about 1-8 weeks, and the third period of time is about 3-6 months, about 4-8 months, or about 6-9 months.

According to another embodiment, the first period of time is between about 2 months and about 6 months, e.g., about 2 months, about 3 months, about 4 months, about 5 months, or about 6 months; and the third period of time is between about 6 months and about 12 months, e.g., about 6 months, about 7 months, about 8 months, about 9 months, about 10 months, about 11 months, or about 12 months. For example, the first period of time is three months and the third period of time is 12 months.

In these embodiments, the first dose, second dose, third dose, and any subsequent dose are the same amount. For example, the dose is 300-2,000 mg. For example, the dose is 500-1,500 mg. For example, the dose is 500-550 mg, 550-600 mg, 600-650 mg, 650-700 mg, 700-750 mg, 750-800 mg, 850-900 mg, 900-50 mg, 950-1,000 mg, 1,000-1,050 mg, 1,050-1,100 mg, 1,100-1,150 mg, 1,150-1,200 mg, 1,200-1,250 mg, 1,250-1,300 mg, 1,300-1,350 mg, 1,350-1,400 mg, 1,400-1,450 mg, 1,450-1,500 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1,000 mg, 1,025 mg, 1,050 mg, 1,075 mg, 1,100 mg, 1,125 mg, 1,150 mg, 1,175 mg, 1,200 mg, 1,225 mg, 1,250 mg, 1,275 mg, 1,300 mg, 1,325 mg, 1,350 mg, 1,375 mg, 1,400 mg, 1,425 mg, 1,450 mg, 1,475 mg, or 1,500 mg. For example, the dose is 600-1,200 mg. For example, the dose is 300-450 mg. For example, the dose is 300 mg. For example, the dose is 400-600 mg. For example, the dose is 400 mg. For example, the dose is 450 mg. For example, the dose is 600 mg. For example, the dose is 700 mg. For example, the dose is 900 mg. For example, the dose is 1,200 mg. For example, the dose is 1,100-1,500 mg. For example, the dose is 600-900 mg. For example, the dose is 700 mg. For example, the dose is 900 mg.

In one embodiment, all doses are administered intravenously. In another embodiment, all doses are administered subcutaneously. In another embodiment, the first dose and second dose are administered intravenously and the third and subsequent doses are administered subcutaneously. Alternatively, a first, second, third, and subsequent doses are 600-1,200 mg, e.g., 600 mg, all administered subcutaneously. Alternatively, a first, second, third and subsequent doses are 600-1,200 mg, e.g., 1,200 mg administered subcutaneously. For example, a first, second, third and subsequent doses are 600-1,200 mg, e.g., 600 mg administered intravenously. For example, a first, second, third, and subsequent doses are 600-1,200 mg, e.g., a first dose of 1,200 mg administered intravenously, followed by second, third and subsequent doses of 1,200 mg administered subcutaneously. For example, a first, second, third and subsequent doses are 600-1,200 mg, e.g., a first and second dose of 600 mg administered intravenously, followed by a third and subsequent doses of 600 mg administered subcutaneously. For example, a first dose of 600-1,200 mg is administered followed by a second dose of 600-1,200 mg about 3 months later, and a third dose of 600-1,200 mg about three months after the second dose, followed by doses about every 6 months after the third dose. The doses may all be 600 mg. For example, the doses may all be 600 mg, administered intravenously for doses 1-2, and subcutaneously for the third and subsequent doses. Alternatively, the doses may all be 1,200 mg. For example, the doses may all be 1,200 mg, administered intravenously for doses 1-2, and subcutaneously for the third and subsequent doses. Alternatively, instead of 600 mg or 1,200 mg in the aforementioned embodiments, the dose is 400-450 mg.

### C. Fixed Doses Administered at Varying Time Intervals

Another embodiment using fixed doses (i.e., doses of the same amount) of an anti-plasma kallikrein antibody, such as STAR-0215, (e.g., every dose is the same) to treat plasma kallikrein dependent disorders, e.g., hereditary angioedema (HAE), involves administering the antibody initially with a first shorter interval between a first and second dose and then changing to a second longer interval between remaining doses. For example, in one embodiment, a first dose of the anti-plasma kallikrein antibody is administered to the subject, and after at first period of time from administering the first dose, a second dose of the anti-plasma kallikrein antibody is administered to the subject, and after a second period of time that is longer than the first period of time, a third dose of the anti-plasma kallikrein antibody is administered to the subject. Thereafter, at intervals equal to the second period of time one or more subsequent doses of the anti-plasma kallikrein antibody are administered to the subject. In these embodiments, the first dose, second dose, third dose and any subsequent dose are fixed doses, i.e., they are the same amount. The dose may be a dose as disclosed herein below.

According to one embodiment, the first period of time may be between about 2 months and about 6 months, e.g., about 2 months, about 3 months, about 4 months, about 5 months, or about 6 months; and the second period of time is between about 4 months and about 9 months, e.g., about 3 months, about 4 months, about 5 months, about 6 months, about 7 months, about 8 months, or about 9 months. Accordingly, in one embodiment, the first period of time may be about two months and the second period of time is about four months, such that the subject receives a dose of the antibody about every four months after the second dose. Alternatively, the first period of time is about three months, and the second period of time is about six months, such that the subject receives a dose of the antibody about every six months after the second dose. Alternatively, the first period of time is about four months and the second period of time is about eight months, such that the subject receives a dose of the antibody about every eight months after the second dose. Alternatively, the first period of time is about four months and the second period of time is about nine months, such that the subject receives a dose of the antibody about every nine months after the second dose. In another embodiment, the first period of time may be between about 1 month and about 3 months, e.g., about 1 month. For example, the first period of time is about one month and the second period of time is about six months, such that the subject receives a dose of the antibody about every six eight months after the second dose. In these embodiments, the first dose, second dose, third dose and any subsequent dose are fixed doses, i.e., they are the same amount. The dose may be a dose as disclosed herein below.

According to another embodiment, the first period of time is between about 2 months and about 6 months, e.g., about 2 months, about 3 months, about 4 months, about 5 months, or about 6 months; and the second period of time is between about 6 months and about 12 months, e.g., about 6 months, about 7 months, about 8 months, about 9 months, about 10 months, about 11 months, or about 12 months. For example, the first period of time is three months and the third period of time is 12 months, such that the subject receives a dose of the antibody about every 12 months after the second dose. The dose may be a dose as disclosed herein below.

In other examples, the first period of time is 2-6 weeks, or 4 weeks±2 weeks. For example, the first period of time is 2 weeks, or the first period of time is 4 weeks, or the first period of time is 6 weeks. In some examples, the second period of time is 3-9 months, or 3-6 months, or 6 months. In one example, the second dose is administered 2-6 weeks after the first dose, the third dose is administered 6 months after the second dose, and subsequent doses are administered at 6 month intervals after the third dose, such that the subject receives a dose of the antibody every 6 months after the second dose. For example, the second dose is administered 4 weeks after the first dose, the third dose is administered 6 months after the second dose, and subsequent doses are administered at 6 month intervals after the third dose, such that the subject receives a dose of the antibody every 6 months after the second dose. In another example, the second dose is administered 2-6 weeks after the first dose, the third dose is administered 5 months after the second dose, and subsequent doses are administered at 5 month intervals after the third dose, such that the subject receives a dose of the antibody every 5 months after the second dose. In all of these embodiments, the first dose, second dose, third dose, and any subsequent dose are the same amount. The dose may be a dose as disclosed herein below.

In some embodiments, the second dose may occur as early as 1 week after the first dose. For example, the first period of time may be 1-4 weeks, e.g., about 1 week, about 2 weeks, about 3 weeks, or about 4 weeks. For example, the first period of time may be about 1 week. In some embodiments, the first period of time may be about 1 week, about 2 weeks, about 3 weeks, or about 4 weeks, e.g., about 1 week, and the second period of time is about 3 months, about 4 months, about 5 months, or about 6 months, e.g., about 3 months. In some embodiments, the first period of time may be about 1-8 weeks, and the second period of time is about 3-6 months, about 4-8 months, or about 6-9 months. In some embodiments, the first period of time may be about 1-2 weeks. In some embodiments, the first period of time may be about 1-2 weeks and the second period of time may be about 3 months. In some embodiments, the first period of time may be about 1-2 weeks and the second period of time may be about 3 months. The dose may be a dose as disclosed herein below.

In one example, a first dose of 300-900 mg is administered, a second dose of 300-900 mg is administered 1-2 weeks after the first dose, and a third dose of 300-900 mg is administered 3-6 months after the second dose, followed by subsequent doses of 300-900 mg administered at 3-6 month intervals after the third dose, such that the subject receives a 300-900 mg dose of the antibody every 3-6 months after the second dose. The first dose, second dose, third dose and any subsequent dose are the same amount. The dose may be a dose as disclosed herein below.

In one example, a first dose of 300-900 mg is administered, a second dose of 300-900 mg is administered 1-2 weeks after the first dose, and a third dose of 300-900 mg is administered 3 months after the second dose, followed by subsequent doses of 300-900 mg administered at 3 month intervals after the third dose, such that the subject receives a 300-900 mg dose of the antibody every 3 months after the second dose. The first dose, second dose, third dose and any subsequent dose are the same amount. The dose may be a dose as disclosed herein below.

In one example, a first dose of 300-900 mg is administered, a second dose of 300-900 mg is administered 1-2 weeks after the first dose, and a third dose of 300-900 mg is administered 6 months after the second dose, followed by subsequent doses of 300-900 mg administered at 6 month intervals after the third dose, such that the subject receives a 300-900 mg dose of the antibody every 6 months after the second dose. The first dose, second dose, third dose and any subsequent dose are the same amount. The dose may be a dose as disclosed herein below.

In one example, a first dose of 600 mg is administered, a second dose of 600 mg is administered 1-2 weeks after the first dose, and a third dose of 600 mg is administered 6 months after the second dose, followed by subsequent doses of 600 mg administered at 6 month intervals after the third dose, such that the subject receives a 600 mg dose of the antibody every 6 months after the second dose.

In one example, a first dose of 400 mg is administered, a second dose of 400 mg is administered 1-2 weeks after the first dose, and a third dose of 400 mg is administered 6 months after the second dose, followed by subsequent doses of 400 mg administered at 6 month intervals after the third dose, such that the subject receives a 400 mg dose of the antibody every 6 months after the second dose.

In one example, a first dose of 400 mg is administered, a second dose of 400 mg is administered 1-2 weeks after the first dose, and a third dose of 400 mg is administered 3 months after the second dose, followed by subsequent doses of 400 mg administered at 3 month intervals after the third dose, such that the subject receives a 400 mg dose of the antibody every 3 months after the second dose.

In one example, a first dose of 600 mg is administered, a second dose of 600 mg is administered 2-6 weeks after the first dose, and a third dose of 600 mg is administered 6 months after the second dose, followed by subsequent doses of 600 mg administered at 6 month intervals after the third dose, such that the subject receives a 600 mg dose of the antibody every 6 months after the second dose.

In another example, a first dose of 600 mg is administered, a second dose of 600 mg is administered 2-6 weeks after the first dose, and a third dose of 600 mg is administered 5 months after the second dose, followed by subsequent doses of 600 mg administered at 5 month intervals after the third dose, such that the subject receives a 600 mg dose of the antibody every 5 months after the second dose.

In one example, a first dose of 700 mg is administered, a second dose of 700 mg is administered 2-6 weeks after the first dose, and a third dose of 700 mg is administered 6 months after the second dose, followed by subsequent doses of 700 mg administered at 6 month intervals after the third dose, such that the subject receives a 700 mg dose of the antibody every 6 months after the second dose.

In another example, a first dose of 1,200 mg is administered, a second dose of 1,200 mg is administered 3 months later, followed by subsequent doses of 1,200 mg administered every twelve months after the second dose. In another example, a first dose of 1,200-1,500 mg is administered, a second dose of 1,200-1,500 mg is administered 3 months later, followed by subsequent doses of 1,200-1,500 mg administered every twelve months after the second dose, where the first second, and subsequent doses are the same.

In some embodiments, for example, the dose is 300-2,000 mg. For example, the dose is 500-1,500 mg. For example, the dose is 300-500 mg. For example, the dose is 300-450 mg. For example, the dose is 300-900 mg. For example, the dose is 500-550 mg, 550-600 mg, 600-650 mg, 650-700 mg, 700-750 mg, 750-800 mg, 850-900 mg, 900-50 mg, 950-1,000 mg, 1,000-1,050 mg, 1,050-1,100 mg, 1,100-1,150 mg, 1, 150-1,200 mg, 1,200-1,250 mg, 1,250-1,300 mg, 1,300-1,350 mg, 1,350-1,400 mg, 1,400-1,450 mg, 1,450-1,500 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1,000 mg, 1,025 mg, 1,050 mg, 1,075 mg, 1,100 mg, 1,125 mg, 1,150 mg, 1,175 mg, 1,200 mg, 1,225 mg, 1,250 mg, 1,275 mg, 1,300 mg, 1,325 mg, 1,350 mg, 1,375 mg, 1,400 mg, 1,425 mg, 1,450 mg, 1,475 mg, or 1,500 mg. For example, the dose is 600-1,200 mg. For example, the dose is 400 mg. For example, the dose is 450 mg. For example, the dose is 600 mg. For example, the dose is 700 mg. For example, the dose is 900 mg. For example, the dose is 1,200 mg. For example, the dose may be 300-900 mg, e.g., 300 mg, 350 mg, 400 mg, 450 mg, 500 mg, 550 mg, 600 mg, 650 mg, 700 mg, 750 mg, 800 mg, 850 mg, or 900 mg. For example, the dose may be 300-400 mg, 400-500 mg, 500-600 mg, 600-700 mg, 700-800 mg, or 800-900 mg.

In one embodiment, all doses are administered intravenously. In another embodiment, all doses are administered subcutaneously. In another embodiment, the first dose is administered intravenously and the second, third, and subsequent doses are administered subcutaneously. In another embodiment, the first and second dose are administered intravenously and the third and subsequent doses are administered subcutaneously. For example, a first, second, third, and subsequent doses are 600-1,200 mg, e.g., 600 mg, all administered subcutaneously. For example, a first, second, third, and subsequent doses are 300-900 mg, all administered subcutaneously. For example, a first, second, third, and subsequent doses are 600-1,200 mg, e.g., 600 mg, all administered intravenously. Alternatively, a first, second, third and subsequent doses are 600-1,200 mg, e.g., 1,200 mg administered subcutaneously. For example, a first, second, third and subsequent doses are 600-1,200 mg, e.g., 1,200 mg administered intravenously. For example, a first, second, third, and subsequent doses are 600-1,200 mg, e.g., a first dose of 1,200 mg administered intravenously, followed by second, third and subsequent doses of 1,200 mg administered subcutaneously. For example, a first, second, third and subsequent doses are 600-1,200 mg, e.g., a first dose of 600 mg administered intravenously, followed by a second, third and subsequent doses of 600 mg administered subcutaneously.

For example, a first dose of 600-1,200 mg is administered followed by 3 months later a second dose of 600-1,200 mg followed by 6 months later a third dose of 600-1,200 mg and a 600-1,200 mg dose every six months thereafter. For example, the dose is 600 mg. For example, the dose is 700 mg. For example, the dose is 900 mg. For example, the dose is 1,200 mg, or the dose may be any other dose disclosed herein.

In some embodiments, administration of the first dose, the second dose, or a subsequent dose maintains a serum concentration of the antibody of greater than about 10 μg/mL for at least 2 months, at least 3 months, at least 4 months, at least 5 months, or at least 6 months. For example, a serum concentration of 12 μg/mL is maintained for about 5 months. For example, a serum concentration of greater than or equal to 25 μg/mL is maintained for about 5 months, e.g., when the doses are 600 mg. For example, a serum concentration of greater than or equal to 12 μg/mL is maintained for about 12 months, e.g., when the doses are 1,200 mg.

In these embodiments, the antibody is STAR-0215, or any of the anti-plasma kallikrein antibodies disclosed herein.

### (ii). Administration in Repeated Treatment Cycles

Exemplary approaches for dosing anti-plasma kallikrein antibodies involving repeated treatment cycles where the cycles include administration of specific doses at specific time intervals are described in the following sections.

### D. Fixed Doses in a Treatment Cycle-Approach 1

In yet another embodiment involving fixed doses of an anti-plasma kallikrein antibody, such as STAR-0215, (e.g., every dose is the same) to treat plasma kallikrein dependent disorders, e.g., hereditary angioedema (HAE), the antibody is administered initially as a first dose, then in repeating treatment cycles of two doses at set intervals. In one example, a method of treating a human subject having a plasma kallikrein associated disorder, e.g., HAE, is disclosed where the subject is administered a first dose of 300-2,000 mg of an anti-plasma kallikrein antibody, followed by administration of one or more subsequent treatment cycles of the antibody where the treatment cycles involve two doses-a first subsequent dose of 300-2,000 mg of the antibody followed by a second subsequent dose of 300-2,000 mg of the antibody. The first subsequent dose is administered after a first period of time from the first dose, or after a third period of time from the second subsequent dose of a previous treatment cycle, and the third period of time is equal to the first period of time. The second subsequent dose is administered after a second period of time from the first subsequent dose. The first period of time is shorter than the second period of time, and the first dose, the first subsequent dose, and the second subsequent doses are the same amount.

In one embodiment, the first period of time (which is the same as the third period of time) is 2-6 weeks. For example, the first period of time (which is the same as the third period of time) is 2 weeks, 3 weeks, 4 weeks, 5 weeks, or 6 weeks, e.g., 4 weeks±2 weeks, e.g., 2 weeks, e.g., 4 weeks, e.g., 6 weeks. The second period of time may be 3-9 months, for example, 3 months, 4 months, 5 months, 6 months, 7 months, 8 months, or 9 months. In one example, the second period of time is 6 months. In another example, the second period of time may be 5 months. In another embodiment, the first period of time may be 2-6 months, e.g., 3 months, and the second period of time may be 6-12 months, e.g., 12 months. In another embodiment, the first period of time (which is the same as the third period of time) is about 1-2 weeks. For example, the first period of time is about 1 week. For example, the first period of time is 2 weeks.

In one particular embodiment, the first subsequent dose is administered 2-6 weeks after the first dose, or 2-6 weeks after the second subsequent dose of a previous treatment cycle, and the second subsequent dose is administered 3-9 months after the first subsequent dose. In another particular embodiment, the first subsequent dose is administered 4 weeks±2 weeks after the first dose, or 4 weeks±2 weeks after the second subsequent dose of a previous treatment cycle, and a second subsequent dose administered 6 months after the first subsequent dose. In another particular embodiment, the first subsequent dose is administered 4 weeks±2 weeks after the first dose, or 4 weeks±2 weeks after the second subsequent dose of a previous treatment cycle, and a second subsequent dose administered 6 months±2 weeks after the first subsequent dose. In yet another particular embodiment, the first subsequent dose is administered 2 weeks after the first dose, or 2 weeks after the second subsequent dose of a previous treatment cycle, and the second subsequent dose is administered 6 months after the first subsequent dose. In yet another particular embodiment, the first subsequent dose is administered about 1 week after the first dose, or about 1 week after the second subsequent dose of a previous treatment cycle, and the second subsequent dose is administered 6 months after the first subsequent dose. In yet another particular embodiment, the first subsequent dose is administered about 1 week after the first dose, or about 1 week after the second subsequent dose of a previous treatment cycle, and the second subsequent dose is administered 3 months after the first subsequent dose. And in yet a further particular embodiment, the first subsequent dose is administered 4 weeks after the first dose, or 4 weeks after the second subsequent dose of a previous treatment cycle, and the second subsequent dose is administered 6 months after the first subsequent dose. Each dose of the antibody may be 600 mg. Each dose of the antibody may be 700 mg. Each dose of the antibody may be 400 mg. The dose may be any dose disclosed herein.

In another particular embodiment, the first subsequent dose is administered 4 weeks±2 weeks after the first dose, or 4 weeks±2 weeks after the second subsequent dose of a previous treatment cycle, and a second subsequent dose administered 5 months after the first subsequent dose. In another particular embodiment, the first subsequent dose is administered 4 weeks±2 weeks after the first dose, or 4 weeks±2 weeks after the second subsequent dose of a previous treatment cycle, and a second subsequent dose administered 5 months±2 weeks after the first subsequent dose. In yet another particular embodiment, the first subsequent dose is administered 2 weeks after the first dose, or 2 weeks after the second subsequent dose of a previous treatment cycle, and the second subsequent dose is administered 5 months after the first subsequent dose. And in yet a further particular embodiment, the first subsequent dose is administered 4 weeks after the first dose, or 4 weeks after the second subsequent dose of a previous treatment cycle, and the second subsequent dose is administered 5 months after the first subsequent dose. Each dose of the antibody may be 600 mg. In yet another embodiment, the first subsequent dose is administered 3 months after the first dose or 3 months after the second subsequent dose of a previous treatment cycle, and the second subsequent dose is administered 12 months after the first subsequent dose. Each of the antibody may be 1,200-1,500 mg, e.g., 1,200 mg.

Alternatively, each dose of the antibody may be 500-1,500 mg, or 600-1,200 mg, e.g., 600 mg, 900 mg or 1,200 mg or any of the following: 500-550 mg, 550-600 mg, 600-650 mg, 650-700 mg, 700-750 mg, 750-800 mg, 850-900 mg, 900-50 mg, 950-1,000 mg, 1,000-1,050 mg, 1,050-1,100 mg, 1,100-1,150 mg, 1,150-1,200 mg, 1,200-1,250 mg, 1,250-1,300 mg, 1,300-1,350 mg, 1,350-1,400 mg, 1,400-1,450 mg, 1,450-1,500 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1,000 mg, 1,025 mg, 1,050 mg, 1,075 mg, 1,100 mg, 1,125 mg, 1,150 mg, 1,175 mg, 1,200 mg, 1,225 mg, 1,250 mg, 1,275 mg, 1,300 mg, 1,325 mg, 1,350 mg, 1,375 mg, 1,400 mg, 1,425 mg, 1,450 mg, 1,475 mg, or 1,500 mg. Each of the doses may be administered subcutaneously.

Alternatively, the doses may be administered intravenously. Yet in another embodiment, the doses may be administered by a combination of intravenous and subcutaneous administration. For example, the first dose and the first subsequent dose may be administered by intravenous administration while the second subsequent dose is administered subcutaneously.

In one embodiment, administration of the first dose followed by a first subsequent dose maintains a serum concentration of the antibody of greater than about 10 μg/mL for at least about 5 months, e.g., 6 months, or, or administration of a second subsequent dose followed by a first subsequent dose maintains a serum concentration of the antibody of greater than about 10 μg/mL for at least about 5 months, e.g., 6 months from the time of administration of the second subsequent dose. For example, a serum concentration of greater than or equal to 12 μg/mL is maintained for at least about 5 months, e.g., 6 months. For example, a serum concentration of greater than or equal to 25 μg/mL is maintained for at least about 5 months, e.g., about 6 months, for example, when the doses are each 600 mg or greater, e.g., 600 mg.

For example, in one embodiment, the first dose is 600 mg followed by one or more treatment cycles consisting of a first subsequent dose of 600 mg administered 4 weeks +2 weeks after the first dose (or after a second subsequent dose of a previous treatment cycle), and a second subsequent dose of 600 mg administered 6 months after the first subsequent dose. The anti-plasma kallikrein antibody may be, e.g., STAR-0215 or may be any, of the anti-plasma kallikrein antibodies disclosed herein. In yet another embodiment, the first dose is 600 mg followed by one or more treatment cycles consisting of a first subsequent dose of 600 mg administered 4 weeks±2 weeks after the first dose (or after a second subsequent dose of a previous treatment cycle), and a second subsequent dose of 600 mg administered 5 months after the first subsequent dose. The anti-plasma kallikrein antibody may be, e.g., STAR-0215 or may be any, of the anti-plasma kallikrein antibodies disclosed herein. In these examples, the subject may have HAE.

### E. Fixed Doses in a Treatment Cycle-Approach 2

In still another embodiment involving fixed doses of an anti-plasma kallikrein antibody, such as STAR-0215, (e.g., every dose is the same) to treat plasma kallikrein dependent disorders, e.g., hereditary angioedema (HAE), a method is disclosed for treating a human subject with a plasma kallikrein associated disorder, e.g., HAE, that involves administering treatment cycles of an anti-plasma-kallikrein antibody, e.g., STAR-0215, or one of the anti-plasma kallikrein antibodies disclosed herein, to the subject. A treatment cycle involves administering a first dose of 300-2,000 mg of the antibody to the subject, then after a first period of time from administering the first dose, administering a second dose of 300-2,000 mg of an anti-plasma kallikrein antibody to the subject. The first and second doses are the same amount. After a second period of time from the second dose, administration of the treatment cycle is repeated one or more times, wherein the second period of time is longer than the first period of time, wherein the first dose and the second dose are the same. The treatment cycle can be administered, for example, one time, two times, three times, four times, five times or six times or more. However, for prophylactic treatment, treatment cycles are repeated indefinitely, e.g., chronically, for example, as long as the subject experiences a clinical benefit.

In one embodiment, the first period of time is 2-6 weeks, for example, 4 weeks±2 weeks. For example, the first period of time may be about 2 weeks, 4 weeks, or 6 weeks. The second period of time may be 3-9 months, for example, about 3 months, about 4 months, about 5 months, about 6 months, about 7 months, about 8 months, or about 9 months. In another embodiment, the second period of time may be 3 months±2 weeks, 4 months±2 weeks, 5 months±2 weeks, 6 months±2 weeks, 7 months±2 weeks, 8 months±2 weeks, or 9 months±2 weeks. For example, the second period of time is 6 months. In another example, the second period of time is 6 months±2 weeks. In another example, the first period of time is 1-2 weeks. For example, the first period of time is about 1 week. For example, the first period of time is about 2 weeks.

In one embodiment, the second dose is administered 2-6 weeks after the first dose and the treatment cycle is repeated 6 months±2 weeks after the second dose. In another embodiment, the second dose is administered 4 weeks±2 weeks after the first dose and the treatment cycles is repeated 6 months±2 weeks after the second dose. In yet another embodiment, the second dose is administered 2 weeks after the first dose and the treatment cycle is repeated 6 months±2 weeks after the second dose. In still another embodiment, the second dose is administered 4 weeks after the first dose, and the treatment cycle is repeated 6 months±2 weeks after the second dose. The treatment cycle may be repeated, for example, 6 months after the second dose.

In one embodiment, the second dose is administered 1-2 weeks after the first dose and the treatment cycle is repeated 3-6 months after the second dose. For example, the second dose is administered about 1 week after the first dose and the treatment cycle is repeated about 6 months±2 weeks after the second dose. For example, the second dose is administered about 1 week after the first dose and the treatment cycle is repeated about 3 months±2 weeks after the second dose. The dose may be any dose disclosed herein. For example, the first and second doses may be 300-900 mg, e.g., 300 mg or 450 mg or 600 mg.

In another embodiment, the second dose is administered 3 months after the first dose and the treatment cycle is repeated 12 months after the second dose. The dose may be 1,200 mg-1,500 mg, e.g., 1,200 mg.

In some embodiments, a treatment cycle lasts about 6 months, e.g., 6 months. For example, in some embodiments, the second dose is administered 2-6 weeks after the first dose and the treatment cycle is repeated 6 months after the first dose, such that the first period of time plus the second period of time equals 6 months., while in other embodiments, the second dose is administered 4 weeks±2 weeks after the first dose and the treatment cycles is repeated 6 months after the first dose, such that the first period of time plus the second period of time equals 6 months. In yet further embodiments, the second dose is administered 2 weeks after the first dose and the treatment cycle is repeated 6 months after the first dose, such that the first period of time plus the second period of time equals 6 months, while in a still further embodiment, the second dose is administered 4 weeks after the first dose, and the treatment cycle is repeated 6 months after the first dose, such that the first period of time plus the second period of time equals 6 months. In some embodiments, the first and second doses may be about 300 mg to about 900 mg, e.g., about 600 mg.

In some embodiments, the second period of time is 5 months, while in other embodiments, the second period of time is 5 months±2 weeks. For example, in some embodiments, the second dose is administered 2-6 weeks after the first dose and the treatment cycle is repeated 5 months±2 weeks after the second dose, while in other embodiments, the second dose is administered 4 weeks±2 weeks after the first dose and the treatment cycles is repeated 5 months±2 weeks after the second dose, while in still further embodiments, the second dose is administered 2 weeks after the first dose and the treatment cycle is repeated 5 months±2 weeks after the second dose. In yet a further embodiment, the second dose is administered 4 weeks after the first dose, and the treatment cycle is repeated 5 months±2 weeks after the second dose. For example, the second dose may be administered 5 months after the second dose. In some embodiments, the first and second doses may be about 300 mg to about 900 mg, e.g. about 600 mg; however, the first and second doses are the same.

In some embodiments, a treatment cycle lasts 7 months. For example, some embodiments, the second dose is administered 2-6 weeks after the first dose and the treatment cycle is repeated 7 months after the first dose, such that the first period of time plus the second period of time equals 7 months, while in still other embodiment, the second dose is administered 4 weeks±2 weeks after the first dose and the treatment cycle is repeated 7 months after the first dose, such that the first period of time plus the second period of time equals 7 months. In further embodiments, the second dose is administered 2 weeks after the first dose and the treatment cycle is repeated 7 months after the first dose, such that the first period of time plus the second period of time equals 7 months, while in other embodiments, the second dose is administered 4 weeks after the first dose, and the treatment cycle is repeated 7 months after the first dose, such that the first period of time plus the second period of time equals 7 months. The first and second doses may be about 600 mg.

According to certain embodiments of the method, administration of a treatment cycle maintains a serum concentration of the antibody of greater than about 10 μg/mL for at least about 5 months from the time of administration of the first dose. For example, in some embodiments, the serum concentration is maintained at greater than or equal to 12 μg/mL for at least 5 months from the administration of the first dose, while in other embodiments the serum concentration is maintained at greater than or equal to 25 μg/mL for at least 5 months from the administration of the first dose. For example, the serum concentration can be maintained for at least 6 months from administration of the first dose.

In some embodiments, the first dose and the second dose of the treatment cycle as disclosed herein are each 500-1,500 mg. For example, the first dose and the second dose may be selected from one of 500-550 mg, 550-600 mg, 600-650 mg, 650-700 mg, 700-750 mg, 750-800 mg, 850-900 mg, 900-50 mg, 950-1,000 mg, 1,000-1,050 mg, 1,050-1,100 mg, 1,100-1,150 mg, 1,150-1,200 mg, 1,200-1,250 mg, 1,250-1,300 mg, 1,300-1,350 mg, 1,350-1,400 mg, 1,400-1,450 mg, 1,450-1,500 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1,000 mg, 1,025 mg, 1,050 mg, 1,075 mg, 1,100 mg, 1,125 mg, 1,150 mg, 1,175 mg, 1,200 mg, 1,225 mg, 1,250 mg, 1,275 mg, 1,300 mg, 1,325 mg, 1,350 mg, 1,375 mg, 1,400 mg, 1,425 mg, 1,450 mg, 1,475 mg, or 1,500 mg. For example, the first dose and the second dose may be 600-1,200 mg. In one embodiment, the first and second dose are each about 600 mg, e.g., 600 mg. In one embodiment, the first and second dose are each about 700 mg, e.g., 700 mg. In yet another embodiment, the first dose and the second dose are each about 900 mg, e.g., 900 mg, while in a further embodiment, the first dose and the second dose are each about 1,200 mg, e.g., 1,200 mg. In some embodiments, the first and second dose are each about 400-600 mg. For example, the doses are 400 mg.

According to the methods disclosed herein, the first dose and the second dose of the treatment cycle may be administered to the subject subcutaneously. Alternatively, they may be administered intravenously. The first dose may be administered intravenously and the second dose may be administered subcutaneously. A subcutaneous dose may be administered from a prefilled syringe, autoinjector device, or via an on-body or wearable injection device, whereas an intravenous dose may be administered as a bolus injection or by infusion.

In one embodiment, the treatment cycle is a first dose of 600 mg administered to the subject followed by administration of a second dose of 600 mg to the subject 4 weeks±2 weeks after the first dose and the treatment cycle is repeated 6 months±2 weeks after administration of the second dose. For example, the second 600 mg dose may be administered 2 weeks after the first 600 mg dose and the treatment cycle repeated 6 months after the first 600 mg dose such that the full treatment cycle is 6 months. For example, the second 600 mg dose may be administered 4 weeks after the first 600 mg dose and the treatment cycle repeated 6 months after the first 600 mg dose such that the full treatment cycle is 6 months. For example, the second 600 mg dose may be administered 6 weeks after the first 600 mg dose and the treatment cycle repeated 6 months after the first 600 mg dose such that the full treatment cycle is 6 months. For example, the second 600 mg dose may be administered 2-6 weeks after the first 600 mg dose and the treatment cycle repeated 6 months after the first 600 mg dose such that the full treatment cycle is 6 months. Alternatively, the doses may be 400 mg instead of 600 mg. In yet another alternative, the doses may be 700 mg instead of 600 mg. However, the first and second doses may be any of the doses disclosed herein.

In other embodiments, the second 600 mg dose may be administered 4 weeks±2 weeks after the first 600 mg dose and the treatment cycle repeated 6 months after the first 600 mg dose such that the full treatment cycle is 6 months. In one embodiment, the treatment cycle is a first dose of 600 mg administered to the subject followed by administration of a second dose of 600 mg to the subject 4 weeks±2 weeks after the first dose and the treatment cycle is repeated 5 months±2 weeks after administration of the second dose. Alternatively, the doses may be 400 mg instead of 600 mg. In yet another alternative, the doses may be 700 mg instead of 600 mg. However, the first and second doses may be any of the doses disclosed herein.

In yet other embodiments, the second 600 mg dose may be administered 2 weeks after the first 600 mg dose and the treatment cycle repeated 7 months after the first 600 mg dose such that the full treatment cycle is 7 months. For example, the second 600 mg dose may be administered 4 weeks after the first 600 mg dose and the treatment cycle repeated 7 months after the first 600 mg dose such that the full treatment cycle is 7 months. For example, the second 600 mg dose may be administered 6 weeks after the first 600 mg dose and the treatment cycle repeated 7 months after the first 600 mg dose such that the full treatment cycle is 7 months. For example, the second 600 mg dose may be administered 2-6 weeks after the first 600 mg dose and the treatment cycle repeated 6 months after the first 600 mg dose such that the full treatment cycle is 7 months. Alternatively, the doses may be 400 mg instead of 600 mg. In yet another alternative, the doses may be 700 mg instead of 600 mg. However, the first and second doses may be any of the doses disclosed herein.

For example, the second 600 mg dose may be administered 4 weeks±2 weeks after the first 600 mg dose and the treatment cycle repeated 7 months after the first 600 mg dose such that the full treatment cycle is 7 months. For example, the second 600 mg dose may be administered 4 weeks±2 weeks after the first dose and the treatment cycle is repeated 6 months±2 weeks after administration of the second dose. For example, the second 600 mg dose may be administered 4 weeks±2 weeks after the first dose and the treatment cycle is repeated 5 months±2 weeks after administration of the second dose. For example, the second 600 mg dose is administered 4 weeks after administration of the first 600 mg dose, and the treatment cycle is repeated 6 months after administration of the second dose. For example, the second 600 mg dose is administered 4 weeks after administration of the first 600 mg dose, and the treatment cycle is repeated 5 months after administration of the second dose. Alternatively, the doses may be 400 mg instead of 600 mg. In yet another alternative, the doses may be 700 mg instead of 600 mg. However, the first and second doses may be any of the doses disclosed herein.

The subject may have HAE in these embodiments.

(iii). Administration of Loading and Maintenance Doses

Exemplary approaches for dosing anti-plasma kallikrein antibodies involving doses (e.g., one or more initial higher doses) followed by maintenance doses (e.g., lower doses) administered at equal or varying time intervals are described in the following section.

### F. Administration of Loading & Maintenance Doses

According to certain methods of treating plasma kallikrein dependent disorders, e.g., hereditary angioedema (HAE) disclosed herein, a loading dose of an anti-plasma kallikrein antibody, such as STAR-0215, is administered followed by one or more maintenance doses. A loading dose provides an initial rapid increase in circulating blood levels or concentrations, e.g., serum or plasma levels of the antibody to exceed a threshold blood concentration required for therapeutically effective pKal inhibition. As the antibody is eliminated from circulation over time, the circulating blood level (e.g., plasma or serum concentration or level) decreases. Accordingly, a certain period of time after administration of the loading dose, e.g., before the blood concentration drops below the threshold blood concentration required for therapeutically effective pKal inhibition, and periodically thereafter, the subject is administered a maintenance dose. The subject may be administered one or more maintenance doses. Chronic administration may be necessary. Maintenance doses are typically remain the same. Accordingly, in some example, the one or more maintenance doses are the same amount.

The first maintenance dose may be administered about 1-6 months after the loading dose. For example, the first maintenance dose may be administered about one month, about two months, about three months, about four months, about five months, or about six months or more, after the loading dose. In one embodiment, the first maintenance dose is administered about three months after the loading dose. In another embodiment, the first maintenance dose is administered about six months after the loading dose. After the first maintenance dose, subsequent maintenance doses may be administered about every one month, about every two months, about every three months, about every four months, about every five months, or about every six months or more, for example, every three months, or every six months in certain embodiments. The first and subsequent maintenance doses are the same amount.

The first maintenance dose may be administered about 1-8 weeks after the loading dose. For example, the first maintenance dose may be administered about 1-4 weeks after the loading dose. For example, the first maintenance dose may be administered about 1-2 weeks after the loading dose. For example, the first maintenance dose may be administered about 2-4 weeks after the loading dose. For example, the first maintenance dose may be administered about 2-6 weeks after the loading dose.

The time period between the loading dose and the first maintenance dose and between the first maintenance dose and subsequent maintenance doses may be the same. For example, the first maintenance and subsequent maintenance doses may be administered at intervals of about one month, about two months, about three months, about four months, about five months, or about six months or more, after the loading dose. For example, in one embodiment, first and subsequent maintenance doses may be administered every 2-6 months, or every 3-6 months. For example, in one embodiment, first and subsequent maintenance doses may be administered in about three month intervals beginning about three months after the loading dose, e.g., every three months after the loading dose. In another embodiment, first and subsequent maintenance doses are administered in about six month intervals beginning about six months after the loading dose, e.g., every six months after the loading dose. In other examples, after the first maintenance dose, subsequent maintenance doses may be administered about every one month, about every two months, about every three months, about every four months, about every five months, or about every six months or more, for example, every three months, or every six months in certain examples. First and subsequent maintenance doses may be the same amount.

Alternatively, the time period between the first maintenance dose and subsequent maintenance doses may be different than the time period between the loading dose and the first maintenance dose. For example, the time period between the loading dose and the first maintenance dose may be from 1 week to 3 months, for example, about 1 week, about 2 weeks, about 3 weeks, about 4 weeks, about 2 months, or about 3 months, and the time period between the first maintenance dose and a second maintenance dose may be 3-9 months, e.g., 3 months, or e.g., 6 months. The first maintenance dose may be administered about 1-8 weeks after the loading dose. For example, the first maintenance dose may be administered about 1-4 weeks after the loading dose. For example, the first maintenance dose may be administered about 1-2 weeks after the loading dose. For example, the first maintenance dose may be administered about 2-4 weeks after the loading dose. For example, the first maintenance dose may be administered about 2-6 weeks after the loading dose.

For example, the time period between the loading dose and the first maintenance dose may be from 1 week to 3 months, and the time period between the first maintenance dose and second maintenance dose may be about 3-9 months, e.g., about 3 months, about 4 months, about 5 months, about 6 months, about 7 months, about 8 months, or about 9 months. The second maintenance dose may even be 12 months after the first maintenance dose.

For example, the first maintenance dose is administered about one week, about two weeks, about three weeks, or about four weeks after the loading dose, and subsequent maintenance doses are administered about every two months, about every three months, about every four months, about every five months, or about every six months or more, for example, every three months, or every six months in certain embodiments. For example, in one embodiment, a first maintenance dose is administered about 1-4 weeks after a loading dose, and subsequent maintenance doses are administered about every 3-6 months, e.g. about every three months, or about every four months, or about every five months, or about every six months after the first maintenance dose. For example, in one embodiment, a first maintenance dose is administered about 1-2 weeks after a loading dose, and subsequent maintenance doses are administered about every three months after the first maintenance dose. For example, in one embodiment, a first maintenance dose is administered about 1-2 weeks after a loading dose, and subsequent maintenance doses are administered about every six months after the first maintenance dose. For example, in one embodiment, a first maintenance dose is administered about 1-8 weeks after a loading dose, and subsequent maintenance doses are administered about every three months, or about every four months, or about every five months, or about every six months after the first maintenance dose. For example, in one embodiment, a first maintenance dose is administered about 2 weeks after a loading dose, and subsequent maintenance doses are administered about every three months after the first maintenance dose. For example, in one embodiment, a first maintenance dose is administered about 2 weeks after a loading dose, and subsequent maintenance doses are administered about every six months after the first maintenance dose. For example, in one embodiment, a first maintenance dose is administered about 4 weeks after a loading dose, and subsequent maintenance doses are administered about every six months after the first maintenance dose.

In one example, a loading dose is administered, a first maintenance dose is administered 1-2 weeks after the loading dose, and a second maintenance dose is administered 3-6 months after the first maintenance dose, followed by subsequent maintenance doses administered at 3-6 month intervals after the second maintenance dose, such that the subject receives a maintenance dose of the antibody every 3-6 months after the first maintenance dose. The interval between maintenance doses is the same. The loading and/or maintenance dose may be any of the loading or maintenance doses disclosed herein. The maintenance dose may be 50% of the loading dose.

In one example, a loading dose of 300-900 mg is administered, a first maintenance dose of 150-700 mg is administered about 1-2 weeks after the loading dose, and a second maintenance dose of 150-700 mg is administered about 3-6 months after the first maintenance dose, followed by subsequent maintenance doses administered at about 3-6 month intervals after the second maintenance dose, such that the subject receives a maintenance dose of the antibody about every 3-6 months after the first maintenance dose. The maintenance dose may be 50% of the loading dose. The loading dose may be 600 mg and the maintenance dose may be 300 mg. The loading dose may be 400 mg and the maintenance dose may be 200 mg. The loading and maintenance dose may be as disclosed herein below. The interval between maintenance doses is the same.

In one example, a loading dose of 300-900 mg is administered, a first maintenance dose of 150-700 mg is administered about 1-2 weeks after the loading dose, and a second maintenance dose of 150-700 mg is administered about 3 months after the first maintenance dose, followed by subsequent maintenance doses administered at about 3 month intervals after the second maintenance dose, such that the subject receives a maintenance dose of the antibody about every 3 months after the first maintenance dose. The maintenance dose may be 50% of the loading dose. The loading dose may be 600 mg and the maintenance dose may be 300 mg. The loading dose may be 400 mg and the maintenance dose may be 200 mg. The loading and maintenance dose may be as disclosed herein below.

In one example, a loading dose of 300-900 mg is administered, a first maintenance dose of 150-700 mg is administered about 1-2 weeks after the loading dose, and a second maintenance dose of 150-700 mg is administered about 6 months after the first maintenance dose, followed by subsequent maintenance doses administered at about 6 month intervals after the second maintenance dose, such that the subject receives a maintenance dose of the antibody about every 6 months after the first maintenance dose. The maintenance dose may be 50% of the loading dose. The loading dose may be 600 mg and the maintenance dose may be 300 mg. The loading dose may be 400 mg and the maintenance dose may be 200 mg. The loading and maintenance dose may be as disclosed herein below.

In one embodiment, a subject having a plasma kallikrein associated disorder, e.g., HAE, is administered a loading dose of the anti-plasma kallikrein antibody with a lower maintenance dose of the antibody administered about every two months thereafter, to treat the disorder. In one embodiment, the loading and maintenance doses are administered subcutaneously. In one embodiment, the loading and maintenance doses are administered intravenously. In yet another embodiment, the loading dose is administered intravenously while the maintenance doses are administered subcutaneously.

In one embodiment, a subject having a plasma kallikrein associated disorder, e.g., HAE, is administered a loading dose of the anti-plasma kallikrein antibody with a lower maintenance dose of the antibody administered about every three months thereafter, to treat the disorder. In one embodiment, the loading and maintenance doses are administered subcutaneously. In one embodiment, the loading and maintenance doses are administered intravenously. In yet another embodiment, the loading dose is administered intravenously while the maintenance doses are administered subcutaneously.

In another embodiment, a subject having a plasma kallikrein associated disorder, e.g., HAE, is administered a loading dose of the anti-plasma kallikrein antibody with a lower maintenance dose of the antibody administered about every four months thereafter, to treat the disorder. In one embodiment, the loading and maintenance doses are administered subcutaneously. In one embodiment, the loading and maintenance doses are administered intravenously. In yet another embodiment, the loading dose is administered intravenously while the maintenance doses are administered subcutaneously.

In another embodiment, a subject having a plasma kallikrein associated disorder, e.g., HAE, is administered a loading dose of the anti-plasma kallikrein antibody with a lower maintenance dose of the antibody administered about every five months thereafter, to treat the disorder. In one embodiment, the loading and maintenance doses are administered subcutaneously. In one embodiment, the loading and maintenance doses are administered intravenously. In yet another embodiment, the loading dose is administered intravenously while the maintenance doses are administered subcutaneously.

In another embodiment, a subject having a plasma kallikrein associated disorder, e.g., HAE, is administered a loading dose of the anti-plasma kallikrein antibody with a lower maintenance dose of the antibody administered about every six months thereafter, to treat the disorder. In one embodiment, the loading and maintenance doses are administered subcutaneously. In one embodiment, the loading and maintenance doses are administered intravenously. In yet another embodiment, the loading dose is administered intravenously while the maintenance doses are administered subcutaneously.

The invention provides that a loading dose and a maintenance dose maintain a blood concentration, e.g., serum concentration of the antibody, e.g., STAR-0215, that exceeds a threshold blood concentration e.g., serum or plasma, concentration of the antibody required for therapeutically effective inhibition of pKal. For example, the threshold blood concentration, e.g., the serum or plasma concentration is 60-80 nM or 8-12 μg/mL. Accordingly, any loading dose with any maintenance dose administered thereafter at periodic intervals that achieves a blood concentration, e.g., a serum or plasma level of the antibody, e.g., STAR-0215, that exceeds a threshold blood concentration, e.g., serum or plasma concentration of the antibody, e.g., 60-80 nM or 8-12 μg/mL is contemplated by the invention.

According to certain methods disclosed herein, the loading dose of the anti-plasma kallikrein antibody, e.g., STAR-0215, may be about 100-600 mg, about 100-700 mg, about 100-800 mg, about 100-900 mg, about 100-1,000 mg, about 100-1,100 mg, about 100-1,200 mg, about 100-1,300 mg, about 100-1,400 mg, about 100-1,500 mg, about 100-1,600 mg, about 100-1,700 mg, about 100-1,800 mg, about 100-1,900 mg, about 100-2,000 mg, about 200-600 mg, about 200-700 mg, about 200-800 mg, about 200-900 mg, about 200-1,000 mg, about 200-1,100 mg, about 200-1,200 mg, about 200-1,300 mg, about 200-1,400 mg, about 200-1,500 mg, about 200-1,600 mg, about 200-1,700 mg, about 200-1,800 mg, about 200-1,900 mg, about 200-2,000 mg, about 300-600 mg, about 300-700 mg, about 300-800 mg, about 300-900 mg, about 300-1,000 mg, about 300-1,100 mg, about 300-1,200 mg, about 300-1,300 mg, about 300-1,400 mg, about 300-1,500 mg, about 300-1,600 mg, about 300-1,700 mg, about 300-1,800 mg, about 300-1,900 mg, about 300-2,000 mg, about 400-600 mg, about 400-700 mg, about 400-800 mg, about 400-900 mg, about 400-1,000 mg, about 400-1,100 mg, about 400-1,200 mg, about 400-1,300 mg, about 400-1,400 mg, about 400-1,500 mg, about 400-1,600 mg, about 400-1,700 mg, about 400-1,800 mg, about 400-1,900 mg, about 400-2,000 mg, about 500-600 mg, about 500-700 mg, about 500-800 mg, about 500-900 mg, about 500-1,000 mg, about 500-1,100 mg, about 500-1,200 mg, about 500-1,300 mg, about 500-1,400 mg, about 500-1,500 mg, about 500-1,600 mg, about 500-1,700 mg, about 500-1,800 mg, about 500-1,900 mg, about 500-2,000 mg, about 600-700 mg, about 600-800 mg, about 600-900 mg, about 600-1,000 mg, about 600-1,100 mg, about 600-1,200 mg, about 600-1,300 mg, about 600-1,400 mg, about 600-1,500 mg, about 600-1,600 mg, about 600-1,700 mg, about 600-1,800 mg, about 600-1,900 mg, about 600-2,000 mg, about 700-800 mg, about 700-900 mg, about 700-1,000 mg, about 700-1,100 mg, about 700-1,200 mg, about 700-1,300 mg, about 700-1,400 mg, about 700-1,500 mg, about 700-1,600 mg, about 700-1,700 mg, about 700-1,800 mg, about 700-1,900 mg, about 700-2,000 mg, about 800-900 mg, about 800-1,000 mg, about 800-1,100 mg, about 800-1,200 mg, about 800-1,300 mg, about 800-1,400 mg, about 800-1,500 mg, about 800-1,600 mg, about 800-1,700 mg, about 800-1,800 mg, about 800-1,900 mg, about 800-2,000 mg, about 900-1,000 mg, about 900-1,100 mg, about 900-1,200 mg, about 900-1,300 mg, about 900-1,400 mg, about 900-1,500 mg, about 900-1,600 mg, about 900-1,700 mg, about 900-1,800 mg, about 900-1,900 mg, about 900-2,000 mg, about 1,000-1,100 mg, about 1,000-1,200 mg, about 1,000-1,300 mg, about 1,000-1,400 mg, about 1,000-1,500 mg, about 1,000-1,600 mg, about 1,000-1,700 mg, about 1,000-1,800 mg, about 1,000-1,900 mg, about 1,000-2,000 mg, about 1,100-1,200 mg, about 1,100-1,300 mg, about 1,100-1,400 mg, about 1,100-1,500 mg, about 1,100-1,600 mg, about 1,100-1,700 mg, about 1,100-1,800 mg, about 1,100-1,900 mg, about 1,100-2,000 mg, about 1,200-1,300 mg, about 1,200-1,400 mg, about 1,200-1,500 mg, about 1,200-1,600 mg, about 1,200-1,700 mg, about 1,200-1,800 mg, about 1,200-1,900 mg, about 1,200-2,000 mg, about 1,300-1,400 mg, about 1,300-1,500 mg, about 1,300-1,600 mg, about 1,300-1,700 mg, about 1,300-1,800 mg, about 1,300-1,900 mg, about 1,300-2,000 mg, about 1,400-1,500 mg, about 1,400-1,600 mg, about 1,400-1,700 mg, about 1,400-1,800 mg, about 1,400-1,900 mg, about 1,400-2,000 mg, about 1,500-1,600 mg, about 1,500-1,700 mg, about 1,500-1,800 mg, about 1,500-1,900 mg, about 1,500-2,000 mg, about 1,600-1,700 mg, about 1,600-1,800 mg, about 1,600-1,900 mg, about 1,600-2,000 mg, about 1,700-1,800 mg, about 1,700-1,900 mg, about 1,700-2,000 mg, about 1,800-1,900 mg, about 1,800-2,000 mg, or about 1,900-2,000 mg. For example, the loading dose may be 600-900 mg in one embodiment, while in another embodiment, the loading dose is 900-1,200 mg. For example, the loading dose may be 900 mg, or the loading dose may be 1,200 mg. In another embodiment, the loading dose is 400 mg-600 mg. In one embodiment, the loading dose may be 600 mg. In another embodiment, the loading dose is 400 mg. The loading dose may be administered in a single dosage form, i.e., a single subcutaneous bolus injection, or a single intravenous infusion or bolus injection. The dose may be administered as two or more sequential subcutaneous injections at the time of administration. In one embodiment, the loading dose is 400 mg administered by a single subcutaneous bolus injection. In one embodiment, the loading dose is 600 mg administered by a single subcutaneous bolus injection. In another embodiment, the loading dose is 900 mg administered by a single subcutaneous bolus injection. In another embodiment, the loading dose is 1,200 mg administered by a single subcutaneous bolus injection. In another embodiment, the loading dose is 150 mg administered every week for one week, two weeks, three weeks, or four weeks subcutaneously or intravenously. In another embodiment, the loading dose is 300 mg administered every week for one week, two weeks, three weeks, or four weeks subcutaneously or intravenously.

According to certain methods disclosed herein, the loading dose of the anti-plasma kallikrein antibody, e.g., STAR-0215, may be about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1,000 mg, about 1,025 mg, about 1,050 mg, about 1,075 mg, about 1,100 mg, about 1,125 mg, about 1,150 mg, about 1,175 mg, about 1,200 mg, about 1,225 mg, about 1,250 mg, about 1,275 mg, about 1,300 mg, about 1,325 mg, about 1,350 mg, about 1,375 mg, about 1,400 mg, about 1,425 mg, about 1,450 mg, about 1,475 mg, about 1,500 mg, about 1,525 mg, about 1,550 mg, about 1,575 mg, about 1,600 mg, about 1,625 mg, about 1,650 mg, about 1,675 mg, about 1,700 mg, about 1,725 mg, about 1,750 mg, about 1,775 mg, about 1,800 mg, about 1,825 mg, about 1,850 mg, about 1,875 mg, about 1,900 mg, about 1,925 mg, about 1,950 mg, about 1,975 mg, or about 2,000 mg. The dose may be administered in a single dosage form, i.e., a single subcutaneous injection, or a single intravenous infusion. The dose may be administered as two or more sequential subcutaneous injections at the time of administration. In one embodiment, the loading dose is 400 mg. For example, 400 mg of the antibody is administered as a loading dose by subcutaneous bolus injection. In one embodiment, the loading dose is 600 mg. For example, 600 mg of the antibody is administered as a loading dose by subcutaneous bolus injection. In one embodiment, the loading dose is 900 mg. For example, 900 mg of the antibody is administered as a loading dose by subcutaneous bolus injection. In one embodiment, the loading dose is 1,200 mg. For example, 1,200 mg of the antibody is administered as a loading dose by subcutaneous bolus injection.

According to certain methods disclosed herein, the maintenance dose of the anti-plasma kallikrein antibody, e.g., STAR-0215, may be about 100-600 mg, about 100-700 mg, about 100-800 mg, about 100-900 mg, about 100-1,000 mg, about 100-1,100 mg, about 100-1,200 mg, about 100-1,300 mg, about 100-1,400 mg, about 100-1,500 mg, about 100-1,600 mg, about 100-1,700 mg, about 100-1,800 mg, about 100-1,900 mg, about 100-2,000 mg, about 200-600 mg, about 200-700 mg, about 200-800 mg, about 200-900 mg, about 200-1,000 mg, about 200-1,100 mg, about 200-1,200 mg, about 200-1,300 mg, about 200-1,400 mg, about 200-1,500 mg, about 200-1,600 mg, about 200-1,700 mg, about 200-1,800 mg, about 200-1,900 mg, about 200-2,000 mg, about 300-600 mg, about 300-700 mg, about 300-800 mg, about 300-900 mg, about 300-1,000 mg, about 300-1,100 mg, about 300-1,200 mg, about 300-1,300 mg, about 300-1,400 mg, about 300-1,500 mg, about 300-1,600 mg, about 300-1,700 mg, about 300-1,800 mg, about 300-1,900 mg, about 300-2,000 mg, about 400-600 mg, about 400-700 mg, about 400-800 mg, about 400-900 mg, about 400-1,000 mg, about 400-1,100 mg, about 400-1,200 mg, about 400-1,300 mg, about 400-1,400 mg, about 400-1,500 mg, about 400-1,600 mg, about 400-1,700 mg, about 400-1,800 mg, about 400-1,900 mg, about 400-2,000 mg, about 500-600 mg, about 500-700 mg, about 500-800 mg, about 500-900 mg, about 500-1,000 mg, about 500-1,100 mg, about 500-1,200 mg, about 500-1,300 mg, about 500-1,400 mg, about 500-1,500 mg, about 500-1,600 mg, about 500-1,700 mg, about 500-1,800 mg, about 500-1,900 mg, about 500-2,000 mg, about 600-700 mg, about 600-800 mg, about 600-900 mg, about 600-1,000 mg, about 600-1,100 mg, about 600-1,200 mg, about 600-1,300 mg, about 600-1,400 mg, about 600-1,500 mg, about 600-1,600 mg, about 600-1,700 mg, about 600-1,800 mg, about 600-1,900 mg, about 600-2,000 mg, about 700-800 mg, about 700-900 mg, about 700-1,000 mg, about 700-1,100 mg, about 700-1,200 mg, about 700-1,300 mg, about 700-1,400 mg, about 700-1,500 mg, about 700-1,600 mg, about 700-1,700 mg, about 700-1,800 mg, about 700-1,900 mg, about 700-2,000 mg, about 800-900 mg, about 800-1,000 mg, about 800-1,100 mg, about 800-1,200 mg, about 800-1,300 mg, about 800-1,400 mg, about 800-1,500 mg, about 800-1,600 mg, about 800-1,700 mg, about 800-1,800 mg, about 800-1,900 mg, about 800-2,000 mg, about 900-1,000 mg, about 900-1,100 mg, about 900-1,200 mg, about 900-1,300 mg, about 900-1,400 mg, about 900-1,500 mg, about 900-1,600 mg, about 900-1,700 mg, about 900-1,800 mg, about 900-1,900 mg, about 900-2,000 mg, about 1,000-1,100 mg, about 1,000-1,200 mg, about 1,000-1,300 mg, about 1,000-1,400 mg, about 1,000-1,500 mg, about 1,000-1,600 mg, about 1,000-1,700 mg, about 1,000-1,800 mg, about 1,000-1,900 mg, about 1,000-2,000 mg, about 1,100-1,200 mg, about 1,100-1,300 mg, about 1,100-1,400 mg, about 1,100-1,500 mg, about 1,100-1,600 mg, about 1,100-1,700 mg, about 1,100-1,800 mg, about 1,100-1,900 mg, about 1,100-2,000 mg, about 1,200-1,300 mg, about 1,200-1,400 mg, about 1,200-1,500 mg, about 1,200-1,600 mg, about 1,200-1,700 mg, about 1,200-1,800 mg, about 1,200-1,900 mg, about 1,200-2,000 mg, about 1,300-1,400 mg, about 1,300-1,500 mg, about 1,300-1,600 mg, about 1,300-1,700 mg, about 1,300-1,800 mg, about 1,300-1,900 mg, about 1,300-2,000 mg, about 1,400-1,500 mg, about 1,400-1,600 mg, about 1,400-1,700 mg, about 1,400-1,800 mg, about 1,400-1,900 mg, about 1,400-2,000 mg, about 1,500-1,600 mg, about 1,500-1,700 mg, about 1,500-1,800 mg, about 1,500-1,900 mg, about 1,500-2,000 mg, about 1,600-1,700 mg, about 1,600-1,800 mg, about 1,600-1,900 mg, about 1,600-2,000 mg, about 1,700-1,800 mg, about 1,700-1,900 mg, about 1,700-2,000 mg, about 1,800-1,900 mg, about 1,800-2,000 mg, or about 1,900-2,000 mg. The dose may be administered in a single dosage form, i.e., a single subcutaneous injection, or a single intravenous infusion. The dose may be administered as two or more sequential subcutaneous injections at the time of administration. The maintenance dose may be half (or 50%) of the amount of the loading dose, e.g., half the mass of the loading dose.

According to certain methods disclosed herein, the maintenance dose of the anti-plasma kallikrein antibody, e.g., STAR-0215, is about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1,000 mg, about 1,025 mg, about 1,050 mg, about 1,075 mg, about 1,100 mg, about 1,125 mg, about 1,150 mg, about 1,175 mg, about 1,200 mg, about 1,225 mg, about 1,250 mg, about 1,275 mg, about 1,300 mg, about 1,325 mg, about 1,350 mg, about 1,375 mg, about 1,400 mg, about 1,425 mg, about 1,450 mg, about 1,475 mg, about 1,500 mg, about 1,525 mg, about 1,550 mg, about 1,575 mg, about 1,600 mg, about 1,625 mg, about 1,650 mg, about 1,675 mg, about 1,700 mg, about 1,725 mg, about 1,750 mg, about 1,775 mg, about 1,800 mg, about 1,825 mg, about 1,850 mg, about 1,875 mg, about 1,900 mg, about 1,925 mg, about 1,950 mg, about 1,975 mg, or about 2,000 mg. The maintenance dose may be half (or 50%) of the amount of the loading dose, e.g., half the mass of the loading dose. The dose may be administered in a single dosage form, i.e., a single subcutaneous injection, or a single intravenous infusion. The dose may be administered as two or more sequential subcutaneous injections at the time of administration. In one embodiment, the maintenance dose is 450 mg. In another embodiment, the maintenance dose is 300 mg. In one embodiment, the maintenance dose is 200 mg. In one embodiment, the maintenance dose is 250 mg. In one embodiment, the maintenance dose is 400 mg. In one embodiment, the maintenance dose is 900 mg.

The loading dose may be about 300-2,000 mg and the maintenance doses may be about 150-1,500 mg. The loading dose may be about 500-2,000 mg and the maintenance doses may be about 250-1,500 mg. The loading dose may be about 400-1,000 mg and the maintenance doses may be about 200-750 mg. The loading dose may be about 600-1,200 mg and the maintenance doses may be about 300-900 mg. The loading dose may be about 900-1,200 mg and the maintenance doses may be about 450-900 mg. The loading dose may be about 600-900 mg and the maintenance doses may be about 300-600 mg. The loading dose may be about 400-600 mg and the maintenance doses may be about 200-300 mg. The loading dose may be 400 mg and the maintenance doses may be 200 mg. The loading dose may be 400 mg and the maintenance doses may be 250 mg. The loading dose may be 600 mg and the maintenance doses may be 300 mg. The loading dose may be 600 mg and the maintenance doses may be 450 mg. The loading dose may be be about 900 mg and the maintenance doses may be about 675 mg. The loading dose may be be about 1,200 mg and the maintenance doses may be about 600 mg. The loading dose may be be about 900 mg and the maintenance doses may be about 675 mg. The loading dose may be be about 900 mg and the maintenance doses may be about 450 mg. The loading and maintenance dose may be administered subcutaneously. The loading dose and the maintenance dose may be administered intravenously. A loading dose in the range of 900-1,200 mg may be administered with maintenance doses in the range of 450-900 mg occurring in six month intervals after the loading dose. For example, the loading dose may be 1,200 mg with a maintenance dose of 900 mg. The loading and maintenance doses may be administered subcutaneously. Alternatively, the loading and maintenance doses may be administered by intravenously, or by a combination of intravenous and subcutaneous administration.

In one embodiment, the loading dose is about 600 mg followed by a maintenance dose of about 300 mg about every 3 months after the loading dose. In another embodiment, the loading dose is about 600 mg, followed by a maintenance dose of about 450 mg about every 3 months after the loading dose. In another embodiment, the loading dose is about 600 mg, followed by a maintenance dose of about 450 mg about every 6 months after the loading dose. In another embodiment, the loading dose is about 600 mg, followed by a maintenance dose of about 400 mg about every 6 months after the loading dose. In another embodiment, the loading dose is about 400 mg, followed by a maintenance dose of about 250 mg about every 3 months after the loading dose. In another embodiment, the loading dose is about 400 mg, followed by a maintenance dose of about 200 mg about every 3 months after the loading dose. In another embodiment, the loading dose is about 1,200 mg, followed by a maintenance dose of about 900 mg about every 6 months after the loading dose. In another embodiment, the loading dose is about 1,200 mg, followed by a maintenance dose of about 600 mg about every 6 months after the loading dose.

The loading dose may also be accomplished by a series of doses over a period of time to achieve a total loading dose that is one of the loading doses disclosed herein. For example, a loading dose of 600 mg may be administered as a 150 mg dose every week for 1, 2, 3, or 4 weeks followed by a maintenance doses every three months thereafter, e.g., in an amount of 300 mg. The loading dose may also be 150 mg every week for 1, 2, 3, or 4 weeks followed by a maintenance doses every three months thereafter, e.g., in an amount of 450 mg. The loading dose may also be 300 mg every week for 1, 2, 3, or 4 weeks followed by a maintenance doses every three months thereafter, e.g., in an amount of 300 mg. The loading dose may also be 300 mg every week for 1, 2, 3, or 4 weeks followed by a maintenance doses every three months thereafter, e.g., in an amount of 450 mg. The loading and maintenance dose may be administered subcutaneously. The loading dose and the maintenance dose may be administered intravenously.

In one embodiment, the loading dose and maintenance dose of the anti-plasma kallikrein antibody, e.g., STAR-0215, is selected from one of the combinations of loading and maintenance doses (e.g., one of combinations 1-43) provided in Table 5 below.

TABLE 5
Exemplary Loading Dose and Maintenance Dose Combinations

| | | | Mainte- | | | | Mainte- | |
|---|---|---|---|---|---|---|---|---|
| Combina- | Loading | | nance | Combina- | Loading | | nance | |
| tion | Dose | | Doses | tion | Dose | | Doses | |
| 1. | 350 | mg | 175 mg | 2. | 1,100 | mg | 875 | mg |
| 3. | 400 | mg | 200 mg | 4. | 1,100 | mg | 550 | mg |
| 5. | 400 | mg | 300 mg | 6. | 1,150 | mg | 575 | mg |
| 7. | 450 | mg | 225 mg | 8. | 1,200 | mg | 600 | mg |
| 9. | 500 | mg | 250 mg | 10. | 1,200 | mg | 900 | mg |
| 11. | 500 | mg | 375 mg | 12. | 1,250 | mg | 625 | mg |
| 13. | 550 | mg | 275 mg | 14. | 1,300 | mg | 650 | mg |
| 15. | 600 | mg | 300 mg | 16. | 1,350 | mg | 675 | mg |
| 17. | 600 | mg | 450 mg | 18. | 1,400 | mg | 700 | mg |
| 19. | 650 | mg | 325 mg | 20. | 1,450 | mg | 725 | mg |
| 21. | 700 | mg | 350 mg | 22. | 1,500 | mg | 750 | mg |
| 23. | 700 | mg | 525 mg | 24. | 1,550 | mg | 775 | mg |
| 25. | 750 | mg | 375 mg | 26. | 1,600 | mg | 800 | mg |
| 27. | 800 | mg | 400 mg | 28. | 1,650 | mg | 825 | mg |
| 29. | 800 | mg | 600 mg | 30. | 1,700 | mg | 850 | mg |
| 31. | 850 | mg | 425 mg | 32. | 1,750 | mg | 875 | mg |
| 33. | 900 | mg | 450 mg | 34. | 1,800 | mg | 900 | mg |
| 35. | 900 | mg | 675 mg | 36. | 1,850 | mg | 925 | mg |
| 37. | 950 | mg | 475 mg | 38. | 1,900 | mg | 950 | mg |
| 39. | 1,000 | mg | 500 mg | 40. | 1,950 | mg | 975 | mg |
| 41. | 1,000 | mg | 750 mg | 42. | 2,000 | mg | 1,000 | mg |
| 43. | 1,050 | mg | 525 mg | 44. | 400 | mg | 250 | mg |

| | | | |
|---|---|---|---|
| 45. | 600 | mg | 400 mg |

In one embodiment, a combination of loading and maintenance doses described in Table 5 (e.g., any one of combinations 1-43 or 44-45) is administered such that a maintenance dose is administered about three months (or 84 days or 12 weeks) after the loading dose and about every three months (or 84 days or twelve weeks) thereafter. In one embodiment, the loading and maintenance doses are administered subcutaneously. In one embodiment, the loading and maintenance doses are administered intravenously. In yet another embodiment, the loading dose is administered intravenously while the maintenance doses are administered subcutaneously. In another embodiment, the loading and maintenance doses are within +/−10% of the stated dose in Table 5.

In one embodiment, a combination of loading and maintenance doses described in Table 5 (e.g., any one of combinations 1-43, or 44-45) is administered such that a maintenance dose is administered about four months (or 112 days or 16 weeks) after the loading dose and about every four months (or 112 days or 16 weeks) thereafter. In one embodiment, the loading and maintenance doses are administered subcutaneously. In one embodiment, the loading and maintenance doses are administered intravenously. In yet another embodiment, the loading dose is administered intravenously while the maintenance doses are administered subcutaneously. In another embodiment, the loading and maintenance doses are within +/−10% of the stated dose in Table 5.

In one embodiment, a combination of loading and maintenance doses described in Table 5 (e.g., any one of combinations 1-43, or 44-45) is administered such that a maintenance dose is administered about five months (or 140-150 days or 20 weeks) after the loading dose and about every five months (or 140 days or 20 weeks) thereafter. In one embodiment, the loading and maintenance doses are administered subcutaneously. In one embodiment, the loading and maintenance doses are administered intravenously. In yet another embodiment, the loading dose is administered intravenously while the maintenance doses are administered subcutaneously. In another embodiment, the loading and maintenance doses are within +/−10% of the stated dose in Table 5.

In one embodiment, a combination of loading and maintenance doses described in Table 5 (e.g., any one of combinations 1-43, or 44-45) is administered such that a maintenance dose is administered about six months (or 160-180 days or 25-26 weeks) after the loading dose and about every six months (or 160-180 days or 25-26 weeks) thereafter. In one embodiment, the loading and maintenance doses are administered subcutaneously. In one embodiment, the loading and maintenance doses are administered intravenously. In yet another embodiment, the loading dose is administered intravenously while the maintenance doses are administered subcutaneously. In another embodiment, the loading and maintenance doses are within +/−10% of the stated dose in Table 5.

In one embodiment, a combination of loading and maintenance doses described in Table 5 (e.g., any one of combinations 1-43, or 44-45) is administered such that a maintenance dose is administered about nine months (or 250-270 days or 36-37 weeks) after the loading dose and about every nine months (or 250-270 days or 36-37 weeks) thereafter. In one embodiment, the loading and maintenance doses are administered subcutaneously. In one embodiment, the loading and maintenance doses are administered intravenously. In yet another embodiment, the loading dose is administered intravenously while the maintenance doses are administered subcutaneously. In another embodiment, the loading and maintenance doses are within +/−10% of the stated dose in Table 5.

In one embodiment, a combination of loading and maintenance doses described in Table 5 (e.g., any one of combinations 1-43, or 44-45) is administered such that a maintenance dose is administered about twelve months (or 336-368 days or 48-52 weeks) after the loading dose and about every twelve months (or 336-368 days or 48-52 weeks) thereafter. In one embodiment, the loading and maintenance doses are administered subcutaneously. In one embodiment, the loading and maintenance doses are administered intravenously. In yet another embodiment, the loading dose is administered intravenously while the maintenance doses are administered subcutaneously. In another embodiment, the loading and maintenance doses are within +/−10% of the stated dose in Table 5.

In one embodiment, a combination of loading and maintenance doses described in Table 5 (e.g., any one of combinations 1-45) is administered such that a first maintenance dose is administered at least about one week after the loading dose, e.g., about 1 week to 8 weeks after the loading dose, and subsequent maintenance doses are administered about every two months, every three months, every four months, every five months, or every six months after the first maintenance dose. For example, the first maintenance dose is administered 1 week after the loading dose and then every three months thereafter. In another embodiment, the first maintenance doses is administered 2 weeks after the loading dose and then every 3-6 months thereafter, e.g., about every 3 months or about every 6 months. In one embodiment, the loading and maintenance doses are administered subcutaneously. In one embodiment, the loading and maintenance doses are administered intravenously. In yet another embodiment, the loading dose is administered intravenously while the maintenance doses are administered subcutaneously. In another embodiment, the loading and maintenance doses are within +/−10% of the stated dose in Table 5.

In one embodiment, a subject diagnosed with a plasma kallikrein associated disorder, e.g., HAE is administered a loading dose of 300-2,000 mg of an anti-plasma kallikrein antibody disclosed herein, e.g., STAR-0215, followed by a maintenance dose of 150-1,500 mg of the antibody beginning at least about 2 months, e.g., about every 3 months, after administering the loading dose and then at least about every two months, e.g., about every 3 months thereafter. In one embodiment, a subject diagnosed with a plasma kallikrein associated disorder, e.g., HAE is administered a loading dose of 500-2,000 mg of an anti-plasma kallikrein antibody disclosed herein, e.g., STAR-0215, followed by a maintenance dose of 250-1,500 mg of the antibody beginning at least about 2 months, e.g., about every 3 months, after administering the loading dose and then at least about every two months, e.g., about every 3 months thereafter. In one embodiment, the loading dose is 600 mg and the maintenance dose is 300 mg, administered subcutaneously, wherein the maintenance dose is administered subcutaneously about 3 months after the loading dose and about every three months thereafter. In one embodiment, the loading dose is 600 mg and the maintenance dose is 450 mg, administered subcutaneously, wherein the maintenance dose is administered about 3 months after the loading dose and about every three months thereafter. In one embodiment, the loading dose is about 600 mg and the maintenance dose is about 400 mg or 450 mg, administered subcutaneously, wherein the maintenance dose is administered about 6 months after the loading dose and about every six months thereafter. In one embodiment, the loading dose is about 400 mg and the maintenance dose is about 250 mg, administered subcutaneously, wherein the maintenance dose is administered about 3 months after the loading dose and about every three months thereafter. In one embodiment, the loading dose is about 400 mg and the maintenance dose is about 200 mg, administered subcutaneously, wherein the maintenance dose is administered about 3 months after the loading dose and about every three months thereafter. The loading dose may be be about 900 mg and the maintenance doses may be about 675 mg. The loading dose may be about 1,200 mg and the maintenance doses may be about 900 mg, wherein the maintenance dose is administered about 6 months after the loading dose and about every six months thereafter. The loading and maintenance dose may be administered subcutaneously. The loading dose and the maintenance dose may be administered intravenously. The anti-plasma kallikrein antibody may be STAR-0215 according to these embodiments.

The antibodies and methods disclosed herein are used to treat a subject who has or is suspected of having a plasma kallikrein associated disorder. The plasma kallikrein associated disorder may be any of hereditary angioedema, bradykinin dependent edema, diabetic macular edema, retinal edema, Factor XII-associated cold autoinflammatory syndrome (FACAS), rheumatoid arthritis, gout, intestinal bowel disease, oral mucositis, neuropathic pain, inflammatory pain, spinal stenosis-degenerative spine disease, arterial or venous thrombosis, post-operative ileus, aortic aneurysm, osteoarthritis, vasculitis, edema, cerebral edema, pulmonary embolism, stroke, clotting induced by ventricular assistance devices or stents, head trauma or peri-tumor brain edema, sepsis, acute middle cerebral artery (MCA) ischemic event (stroke), restenosis (e.g., after angioplasty), systemic lupus erythematosus nephritis, Alzheimer's disease, and burn injury. In one embodiment, the plasma kallikrein associated disorder treated according to the methods disclosed herein is FACAS.

The Angioedema Quality of Life Questionnaire (AE-QoL) (Weller et al., 2012, Allergy, 67 (10); 1289-1298) is a validated, angioedema-specific, patient-reported outcome measure that is self-administered and used to evaluate the impact of recurrent angioedema on patients' quality of life. It is a 17-item questionnaire covering 4 domains (functioning, fatigue/mood, fears/shame, and nutrition). Each item has 5 answer options (scored 1 to 5), with higher scores indicating a more adverse effect. Raw scores are transformed into a linear scale that ranges from 0 to 100, with a score of 100 indicating the worst possible impairment in quality of life. The minimally clinically important difference is defined as a change of 6 points from baseline. The baseline score is the score determined prior to beginning treatment according to the methods disclosed herein. Accordingly, subjects treated according to the methods disclosed herein may attain an improvement in AE-QoL score of at least 6 points from baseline (i.e., the score decreases by at least 6 points). In some embodiments, the improvement in AE-QoL score is at least 6 points from baseline, at least 10 points from baseline, at least 15 points from baseline, at least 20 points from baseline, at least 25 points from baseline, at least 30 points from baseline, at least 35 points from baseline, at least 40 points from baseline, at least 45 points from baseline, or at least 50 points from baseline. The subject may have HAE.

In some embodiments according to the invention, a subject treated according to the methods disclosed herein experiences a decrease in the number of attacks, e.g., HAE attacks as compared to a baseline. In some embodiments, the baseline number of attacks is 1-4 attacks per month. For example, the baseline number of attacks is about 2 attacks per month. According to the methods disclosed herein, subjects may experience a 75-90% decrease in frequency of attacks per month (monthly attack rate) compared to baseline, which is based on the attack rate prior to initiation (onset) of the treatment. The decrease in frequency of attacks per month from baseline in a subject may even be as high as 75-100% reduction in frequency of attacks per month. For example, the decrease in the monthly attack rate for the subject may be 80-100%, 85-100%, or 90-100% as compared to baseline when the subject is treated with an antibody as disclosed herein, e.g., STAR-0215, such as according to the dosing regimens disclosed herein, e.g., a dosing regimen disclosed in Example 5 for Cohort 1, Cohort 2, or Cohort 3. For example, the decrease in monthly attack rate for the subject may be 85-90%, 85-95%, 86-95%. 87-95%, 88-95%, 89-95%, 90-95%, 91-95%, 92-95%, 93-95%, 94-95%, 85-100%, 86-100%, 87-100%, 88-100%, 89-100%, 90-100%, 91-100%, 92-100%, 93-100%, 94-100%, or 95-100% as compared to baseline. For example, the decrease in monthly attack rate for the subject may be 90-95%, 90-96%, 90-97%, 90-98%, 90-99% as compared to baseline. For example, the decrease in monthly attack rate for the subject may be about 85%, about 86%, about 87%, about 88%, about 89%, about 90%, about 91%, about 92%, about 93%, about 94%, about 95%, about 96%, about 97%, about 98%, or about 99% as compared to baseline. In some instances, the subject may be attack-free compared to baseline (e.g., the subject experiences a 100% reduction in the monthly attack rate compared to baseline). These decreases in monthly attack rate may occur as a result of treatment with STAR-0215 as disclosed herein, for example, according to a dosing regimen outlined in Example 5 herein. These decreases in monthly attack rate may occur for at least three months, e.g., six months, from commencing treatment, e.g., treatment with STAR-0215, for example, according to a dosing regimen disclosed in Example 5 herein. Baseline may be determined by assessing the number of attacks for a period of time prior to treatment, e.g., for four weeks prior to treatment, for eight weeks prior to starting the treatment, or for twelve weeks prior to starting treatment. A comparison to baseline may involve a period of time from the subject starting treatment that is the same length of time or longer than the baseline period of time to determine if an improvement over baseline has occurred while the patient is receiving treatment with the anti-plasma kallikrein antibody, or it can be assessed on a “per month” basis.

In some embodiments according to the invention, subjects treated according to the methods disclosed herein experience a decrease in the severity of attacks, e.g., HAE attacks, as compared to a baseline. Baseline may be determined by assessing the severity of attacks for a period of time prior to treatment, e.g., for four weeks, eight weeks or twelve weeks prior to starting the treatment. A comparison to baseline may involve a period of time from the subject starting treatment that is the same length of time or longer than the baseline period of time to determine if an improvement over baseline has occurred while the patient is receiving treatment with the anti-plasma kallikrein antibody, or it can be assessed on a monthly basis, e.g., the level of severity of attacks on average over a month prior to treatment versus a month receiving treatment. The baseline may be determined by calculating a monthly average of severity of attacks, e.g., a monthly rate of severe attacks and/or a monthly rate of moderate attacks and/or a monthly rate of mild attacks, and then comparing that to the monthly rate of attacks of the same severity after receiving treatment. Severity of attacks may be assessed as mild, moderate, or severe according to the following criteria: Mild: transient or mild discomfort; Moderate: mild to moderate limitation in activity, some assistance with daily activities needed; Severe: marked limitation in activity, assistance with daily activities required. The decrease in monthly attack rate for moderate and/or severe attacks resulting from treatment according to the methods disclosed herein may be 85-100%, 85-95%, 85-90%, 90-95%, 95-100%, or 90-100% as compared to baseline. The decrease in monthly attack rate for moderate and/or severe attacks may be 90-100% as compared to baseline when the subject is treated with an antibody as disclosed herein, e.g., STAR-0215, such as according to the dosing regimens disclosed herein, for example, dosing regimens according to Example 5 for Cohort 1, Cohort 2, or Cohort 3. For example, the decrease in monthly attack rate for moderate and/or severe attacks in the subject may be 91-100%, 92-100%, 93-100%, 94-100%, 95-100%, or 96-100% as compared to a baseline. For example, the decrease in monthly attack rate for moderate and/or severe attacks for the subject may be 90-95% as compared to a baseline. For example, the decrease in monthly attack rate for moderate and/or severe attacks in the subject may be at least about 85%, about 86%, about 87%, about 88%, about 89%, about 90%, about 91%, about 92%, about 93%, about 94%, about 95%, about 96%, about 97%, about 98%, or about 99% as compared to baseline. In some instances, the subject may be severe attack-free compared to baseline (e.g., the subject experiences a 100% reduction in the severe monthly attack rate compared to baseline) after commencing treatment with the anti-plasma kallikrein antibody, e.g., STAR-0215. These decreases in severe and/or moderate monthly attack rate may occur as a result of treatment with STAR-0215, for example, according to a dosing regimen disclosed herein, such as in Example 5 herein, and may occur for at least three months or at least six months from commencing treatment, e.g., treatment with STAR-0215 as disclosed herein, for example, as disclosed in Example 5 herein.

In some embodiments according to the invention, a subject treated according to the methods disclosed herein experiences a decrease in the number of attacks, e.g., HAE attacks requiring use by the subject of an on-demand or “rescue” medication to treat the attack as compared to a baseline. The baseline may be calculated by determining the number of attacks per month that required use of on-demand therapy by the subject prior to the subject beginning treatment with an antibody according to the methods of the invention, e.g., STAR-0215, such as according to a dosing regimen disclosed herein, for example a dosing regimen of Example 5 herein. According to the methods disclosed herein, subjects may experience at least an 85% decrease in frequency of attacks per month requiring rescue medication compared to baseline, but the decrease may be as high as 100%. For example, the decrease in the frequency of attacks per month requiring rescue medication may be 85-100%, e.g., 90-100%, as compared to baseline when the subject is treated with STAR-0215, such as according to the dosing regimens disclosed herein, for example, the dosing regimens in Example 5 for Cohort 1, Cohort 2, or Cohort 3, where the decrease is over a 3 month period from beginning treatment with STAR-0215, or is over a 6 month period from beginning treatment with STAR-0215. For example, the decrease in the frequency of attacks per month requiring rescue medication for the subject may be 85-90%, 85-95%, 91-100%, 92-100%, 93-100%, 94-100%, or 95-100% as compared to baseline. For example, the in the frequency of attacks per month requiring rescue medication for the subject may be 90-95% as compared to baseline. For example, the decrease in the frequency of attacks per month requiring rescue medication for the subject may be about 85%, about 86%, about 87%, about 88%, about 89%, about 90%, about 91%, about 92%, about 93%, about 94%, about 95%, about 96%, about 97%, about 98%, or about 99% as compared to baseline. In some instances, the subject may experience no attacks requiring rescue medication compared to baseline (e.g., the subject experiences a 100% reduction in the number of monthly attacks requiring rescue medication compared to baseline). These decreases in the number of monthly attacks requiring rescue medication may occur as a result of treatment with STAR-0215, for example, according to a dosing regimens outlined in Example 5 herein. These decreases in the number of monthly attacks requiring rescue medication may occur for at least three months from commencing treatment, e.g., treatment with STAR-0215 as disclosed in Example 5 herein. The decrease in the number of monthly attacks requiring rescue medication may occur for at least six months from commencing treatment, e.g., treatment with STAR-0215 as disclosed in Example 5 herein.

### 4. Pharmaceutical Compositions and Modes of Administration

The anti-plasma kallikrein antibodies disclosed herein can be formulated as pharmaceutical compositions, e.g., for administration to a subject. As used herein, the term “pharmaceutical composition” refers to the combination of an active agent, e.g., the anti-plasma kallikrein antibodies described herein, with a carrier, inert or active, making the composition especially suitable for diagnostic or therapeutic use in vivo or ex vivo.

Pharmaceutical compositions containing antibodies can be presented in a dosage unit form and can be prepared by any suitable method. In some embodiments, to prepare pharmaceutical or sterile compositions of the anti-plasma kallikrein antibodies, the antibody or antigen binding fragment thereof or other proteins provided herein are admixed with a pharmaceutically acceptable carrier or excipient. A pharmaceutical composition should be formulated to be compatible with its intended route of administration, e.g., subcutaneous or intravenous administration. Useful formulations can be prepared by methods known in the pharmaceutical art. For example, see Remington's Pharmaceutical Sciences, 18th ed. (Mack Publishing Company, 1990).

The anti-plasma kallikrein antibodies administered according to the methods disclosed herein can be formulated into pharmaceutical compositions for administration according to the skill in the art. Pharmaceutical compositions containing the antibodies may be formulated with pharmaceutically acceptable buffers (e.g., amino acid buffers, citrate buffers, succinate buffers, acetate buffers, phosphate buffers, etc.), stabilizers ((e.g., amino acids such as arginine, glycine, lysine, histidine, glutamic acid, aspartic acid, isoleucine, leucine, alanine, phenylalanine, tyrosine, tryptophane, methionine, serine, proline, or cysteine), or sugars (e.g., polyols, monosaccharides, disaccharides, or oligosaccharides, such as mannitol, sorbitol, glucose, fructose, mannose, sorbose, galactose, trehalose, maltose, raffinose, etc.)), tonicifiers (e.g., salts or sugars), surfactants (e.g., ionic surfactants (carboxylates, sulfates, sulfonates, phosphate esters) or ionic surfactants (e.g., polysorbates or polyoxyethylenes (Tweens), polaxamers, or sorbitans (Spans)), and diluents (e.g., sterile water, phosphate-buffered saline etc.), among other pharmaceutically acceptable excipients suitable for use in the formulations of antibodies. The formulation should be suitable for administration to a human subject by the desired mode of administration, e.g., by subcutaneous or intravenous injection. For example, a formulation for subcutaneous administration may include a dispersion enhancer to support dispersion of a subcutaneously injected volume. In one embodiment, the antibodies disclosed herein may be formulated at a concentration of 150 mg/mL in sterile solution at a pH of 5.8 with histidine, L-methionine, sorbitol, glycine, and polysorbate 80. For example, the formulation of the antibodies includes 10 mM histidine-HCl, 75 mM glycine, 10 mM L-methionine, 150 mM sorbitol, and 0.05% w/v polysorbate 80 at pH 5.8.

In some embodiments, the anti-plasma kallikrein antibodies administered according to the methods disclosed herein are formulated in a pharmaceutical composition at concentration of 100-200 μg/mL, e.g., the concentration may be 100-150 μg/mL or 150-200 μg/mL. For example the concentration is about 100 μg/mL, about 105 μg/mL, about 110 μg/mL, about 115 μg/mL, about 120 μg/mL, about 125 μg/mL, about 130 μg/mL, about 135 μg/mL, about 140 μg/mL, about 145 μg/mL, about 150 μg/mL, about 155 μg/mL, about 160 μg/mL, about 165 μg/mL, about 170 μg/mL, about 175 μg/mL, about 180 g/mL, about 185 μg/mL, about 190 μg/mL, about 195 μg/mL, or about 200 μg/mL. In one embodiment, the concentration of the antibody is 150 μg/mL.

For intravenous administration, suitable carriers include physiological saline, bacteriostatic water, Cremophor EL™ (BASF, Parsippany, NJ), phosphate buffered saline (PBS) or dextrose solution. The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyethylene glycol), and suitable mixtures thereof.

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

Formulations of the antibodies provided herein may be prepared by mixing with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie ( ) Excipient Toxicity and Safety, Marcel Dekker, Inc., New York, NY).

Formulations of antibodies may be filled in a fixed dose into a vial; a syringe fitted with a needle; a syringe without a pre-fitted needle (where the person administering the antibody adds the needle prior to administration); into an autoinjector pen; into a cartridge for an autoinjector pen

Toxicity and therapeutic efficacy of the antibody compositions, administered alone or in combination with another agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). In particular aspects, antibodies exhibiting high therapeutic indices are desirable. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

Antibody compositions disclosed herein may be administered to a subject, for example, in accordance with the Physicians' Desk Reference 2003 (Thomson Healthcare; 57th edition (Nov. 1, 2002)).

The mode of administration can vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial.

According to one embodiment, antibodies are administered parenterally according to the methods disclosed herein.

A preferred route of administration according to the methods disclosed herein is subcutaneous administration. The anti-plasma kallikrein antibodies disclosed herein may be administered subcutaneously via a prefilled syringe fitted, e.g., fitted with a needle, for example a 23-27 gauge ⅝ inch needle. Alternatively, the antibodies may be administered subcutaneously via a prefilled autoinjector pen, or via a prefilled cartridge inserted into an autoinjector pen. The autoinjector may be an electronic autoinjector. The antibody formulation may be contained within a vial where the dose is removed from the vial and injected subcutaneously with a needle, for example a 23-27 gauge ⅝ inch needle. Alternatively, the antibodies can be administered subcutaneously via a wearable or “on-body” device loaded with the dose. In some embodiments, the antibody or antigen binding fragment thereof can be administered by an invasive route such as by injection. Subcutaneous injection may be to the subject's thigh, buttock, or abdomen, for example. In some embodiments, the subcutaneous injection is performed by the subject, i.e., the antibody is self-administered.

The antibodies disclosed herein may also be administered intravenously, for example, by intravenous infusion, such as drip infusion or pump infusion. Alternatively, the antibodies disclosed herein may be administered via intravenous push or bolus injection via a port or catheter. Infusion may be effected through a port or catheter placed in a vein of the patient. The port or catheter may be a PICC line, tunneled catheter, or implanted port, for example. In some embodiments, the intravenous administration is performed by a medical professional, whereas in some embodiments, the administration is performed by the subject, i.e., the antibody is self-administered. A fixed dose of the antibody may be provided into the port or catheter, or may be provided into a delivery solution for drip administration.

In some embodiments, the anti-plasma kallikrein antibody, or antigen binding fragment thereof, is administered in combination with at least one additional therapeutic agent, such as, but not limited to any therapeutic used to treat the disorders provided for herein. In some embodiments, the antibody is administered in combination with another treatment for the disorders provided for herein. For example, the anti-plasma kallikrein antibody of the invention may be administered with one or more additional therapies used to treat HAE, such as BERINERT® (C1 esterase inhibitor), FIRAZY® (icatibant injection), KALBITOR® (ecallantide), and RUCONEST® (C1 esterase inhibitor, contestat alfa), CINRYZE® (C1 esterase inhibitor), HAEGARDA® (C1 esterase inhibitor subcutaneous), TAKHZYRO® (lanadelumab-flyo), ORLADEYO™ (berotralstat), garadacimab, donidalorsen, synthetic 17-α-alkylated androgens such as danazol and stanozolol, and/or antifibrinolytic agents such as aminocaproic acid. The anti-plasma kallikrein antibody may be administered simultaneously with one or more aforementioned additional therapies, e.g., they may be co-administered. The one or more aforementioned additional therapies may be a chronic therapy (i.e., chronically administered), or an on-demand or “rescue” therapy (i.e., administered to treat an attack). The anti-plasma kallikrein antibody may be administered sequentially, either before or after, one of the aforementioned therapies, for example, on different days or different weeks or even in different months than any of the aforementioned additional therapies.

Pharmaceutical compositions containing anti-plasma kallikrein antibodies can be administered with medical devices known in the art. For example, a pharmaceutical composition disclosed herein can be administered by injection with a hypodermic needle, including, e.g., a prefilled syringe or autoinjector. The pharmaceutical compositions may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556. The pharmaceutical compositions may also be administered by infusion. Examples of well-known implants and modules form administering pharmaceutical compositions include: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art. Infusion may also be carried out by intravenous delivery of an antibody in a pharmaceutically acceptable carrier, such as physiologic saline, over a defined time period. Intravenous delivery of the pharmaceutical composition may occur via a port implanted into a vein or artery in the subject.

The methods described herein can be used alone or in combination with other therapeutic methods. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

Unless defined otherwise, all technical and scientific terms used herein have the same meaning as commonly understood by one of ordinary skill in the art to which this invention belongs.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise. The expression “and/or” in connection with three or more recited objects should be understood to have the same meaning unless otherwise understood from the context.

It should be understood that the expression “at least one of” includes individually each of the recited objects after the expression and the various combinations of two or more of the recited objects unless otherwise understood from the context and use.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the disclosure also includes the specific quantitative value itself, unless specifically stated otherwise. As used herein, the term “about” refers to a ±10% variation from the nominal value unless otherwise indicated or inferred. For example, “about 10” contemplates both 10 and 10±10%.

The term “in combination with” as used herein means that the described agents can be administered to an animal or subject together in a mixture, concurrently as single agents, or sequentially as single agents in any order.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As a general matter, compositions specifying a percentage are by weight unless otherwise specified. Further, if a variable is not accompanied by a definition, then the previous definition of the variable controls.

It should be understood that the order of steps or order for performing certain actions is immaterial so long as the present invention remain operable. Moreover, two or more steps or actions may be conducted simultaneously.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The subject matter is now described with reference to the following examples. These examples are provided for the purpose of illustration only and the claims should in no way be construed as being limited to these examples, but rather should be construed to encompass any and all variations which become evident as a result of the teaching provided herein. Those of skill in the art will readily recognize a variety of non-critical parameters that could be changed or modified to yield essentially similar results.

### EXAMPLES

### Example 1. STAR-0215 Inhibits pKal-Mediated Cleavage of HMWK in Cynomolgus Monkey

The presence of cleaved high molecular weight kininogen (cHMWK) in plasma is a potential endogenous physiologic pharmacodynamic (PD) marker for STAR-0215 pKal inhibitory activity in normal cynomolgus monkeys. Western blotting analysis was used to evaluate the presence of cHMWK in plasma samples following subcutaneous (SC) dosing of STAR-0215 at 10, 30, or 100 mg/kg. As cynomolgus monkeys have low, non-pathologic levels of plasma kallikrein and therefore low levels of cHMWK, ex vivo activation of plasma by an exogenous trigger, such as activated FXIIa, is required to generate high levels of cHMWK. This methodology has previously been utilized during the early development of other plasma kallikrein inhibitors (See, e.g., Banerji et al., 2017, N Engl J Med, 376:717-728; Kenniston et al., 2014, J. Biol Chem, 289:23596-23608).

Animals were administered STAR-0215 or vehicle control (10 mM histidine-HCl, 75 mM glycine, 10 mM L-methionine, 150 mM sorbitol, 0.05% w/v PS80 at pH 5.8) via SC at 10, 30, or 100 mg/kg with an 84-day dose-free period in between doses. Plasma samples were collected using 3.2% Na-Citrate tubes and quickly aliquoted and snap-frozen. Samples were thawed on ice, and were either left untreated or treated with active human Factor XII (hFXIIa) at a final concentration of 10 nM for 10 min at 37° C. to simulate activation of the contact pathway to induce the activation of PK, and the formation of pKal.

To monitor the enzyme reaction, a TQC control (a pool of cynomolgus monkey plasma samples with no added protease inhibitor), was also treated with hFXIIa exactly as the samples being treated in each run. After treatment, both hFXIIa-treated and untreated samples were added to a protease inhibitor cocktail. All samples, including High Positive Control (HQC; 100% cHMWK), Mid Positive Control (MQC; 60% cHMWK), Low Positive Control (LQC; 15% cHMWK) and treated TQC samples were further diluted (1:20) in ice-cold 1× PBS with protease inhibitors. Samples were loaded onto Novex™ 4 to 20% Tris-Glycine Protein Gels (ThermoFisher) for SDS-PAGE electrophoresis. Proteins were transferred onto nitrocellulose membranes, blocked with Intercept® (TBS) Blocking Buffer (LI-COR, Lincoln, NE) for 1 hr. This was followed by incubation with an anti-kininogen/kininostatin antibody (0.1 ng/μL; R&D Systems) overnight at 4° C. Membranes were washed 4 times with 1× TBST (20 mM Tris, 150 mM NaCl, 0.1% Tween-20). Infrared-dye-labeled secondary antibody (IRDye® 800CW Donkey anti-Goat IgG) was added at 1:10,000 dilution. The membranes were washed again and scanned on a LI-COR Odyssey scanner to obtain blot images. The signal intensities of single-chain (approximately 110 kDa) and two-chain HMWK bands (approximately 45 and 55 kDa) were analyzed using LI-COR Odyssey software and the ratios of two-chain HMWK over the total HMWK in the absence (% cleavage) and presence of 10 nM of FXIIa treatment (% cleavage under 10 nM FXIIa) were reported for each sample as percentages.

The Western blotting method can distinguish between HMWK and cHMWK and allows for quantitation of both forms. Because the ex vivo activation mediated production of cHMWK for pre-dose samples and was variable between animals, the data were normalized as percent change from pre-dose on an animal-by-animal basis.

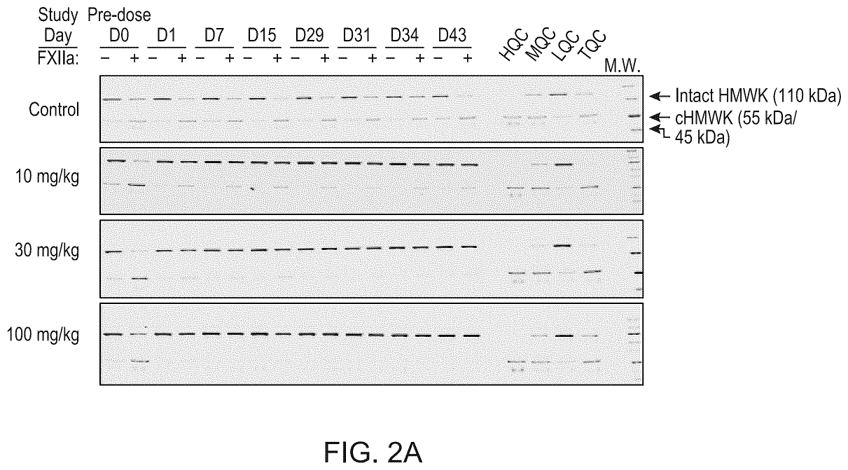

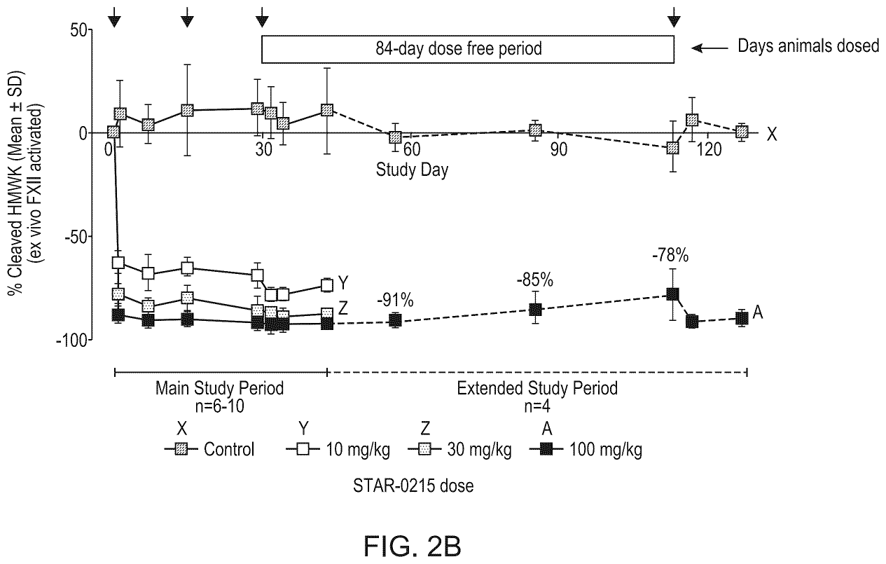

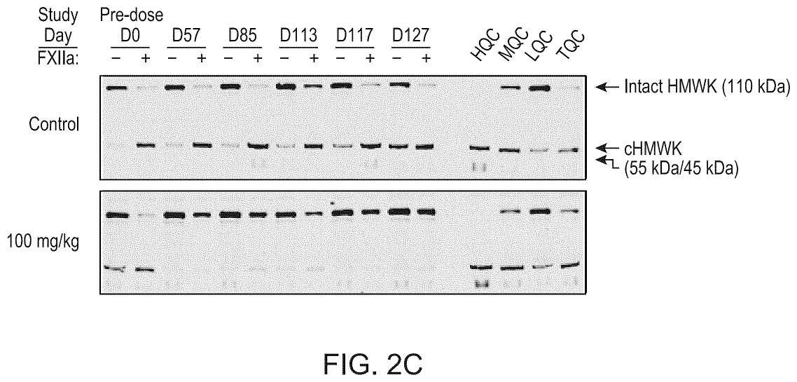

The Western Blots provided in FIGS. 2A and 2C show intact (the endogenous pKal substrate) and cleaved HMWK pre-dose at specific time points after dosing with STAR-0215 on Day 1, Day 14, Day 29, with and without treatment with FXIIa (main study period). High Positive Control (HQC), Mid Positive Control (MQC), and Low Positive Control (LQC) are pre-aliquoted cynomolgus plasma samples with approximately 99, 67, and 18% cHMWK, respectively visible on the right side of each of the blots. TQC is an aliquot of the LQC sample treated with FXIIa as an additional treatment control. FIG. 2A shows Western blots of intact HMWK and cHMWK from control animals and animals dosed with STAR-0215 at 10, 30, and 100 mg/kg up to day 43. FIG. 2B is an analysis of STAR-0215 inhibitory activity (% cHMWK) from both the main study period (days 0-43) derived from FIG. 2A and the extended study period (days 44-127) with an additional dose given on Day 113. Representative Western blots from control and animals dosed with STAR-0215 (100 mg/kg) up to day 127 are shown in FIG. 2C. The signal intensities of single-chain (approximately 110 kDa) and two-chain HMWK (cHMWK) bands (approximately 45 and 55 kDa) were analyzed using LI-COR Odyssey software and the ratios of two-chain HMWK over the total HMWK in the absence (% cleavage) and presence of 10 nM of FXIIa treatment were reported for each sample as percentages. n=6-10 for main study period, and n=4 for extended study period.

The percent change in cHMWK from the pre-dose values in ex vivo FXIIa activated cHMWK in post-dose plasma samples compared to the pre-dose values demonstrated a clear PD effect, with a strong inhibition of cHMWK observed with all doses of STAR-0215 (10, 30 and 100 mg/kg) (FIGS. 2A and 2B). The greatest inhibition of cHMWK compared with pre-dose values was observed at the 100 mg/kg dose level (FIGS. 2B and 2C). At the two lowest dose levels of STAR-0215 inhibition of cHMWK was sustained for the duration of the study, including an 84-day dose-free period (between doses on Day 29 and Day 113).

This demonstrates that STAR-0215 Inhibits pKal Mediated Cleavage of HMWK in a cynomolgus monkey, but more importantly, it demonstrates that long-lasting inhibition of pKal is achievable after a single subcutaneous dose of STAR-0215.

### Example 2. Pharmacokinetic Modeling to Suggest STAR-0215 Exposures Required to Suppress HAE Attacks

A minimum physiologically based pharmacokinetic (mPBPK) model was established using the data from the cynomolgus PK study. Simulations were performed to determine the exposure of STAR-0215 required to achieve target exposure level in humans. The human PK profiles of STAR-0215 at a single subcutaneous dose of 100, 300, and 600 mg over a period of 3 months was simulated for a typical 70-kg healthy subject.

The mPBPK model and simulations were performed using Phoenix WinNonlin (Version 8.3). The model uses physiological constants and inputs from the literature (Cao et al., J Pharmacokinet Pharacodyn, 40:597-607; Offman et al., 2016, In Silico Pharmacol, 4:3). The mPBPK model from Cao et al. was implemented in WinNonlin for IV exposure and modified as per Offman et al. for subcutaneous exposure. The mPBPK model was fit to the IV data to estimate chemical specific PK parameters (i.e., non-specific plasma clearance). Then, the IV mPBPK parameters was used to estimate the SC specific mPBPK model parameters (related to absorption rate and bioavailability) using the SC data. The reference body weight was based on the actual measured weight in animal studies. Human model was obtained by substituting the monkey physiological parameters with the literature human values. All other parameters (bioavailability and tissue refraction coefficients) were using model fitted values. Human model simulation was based on a typical 70 kg body weight.

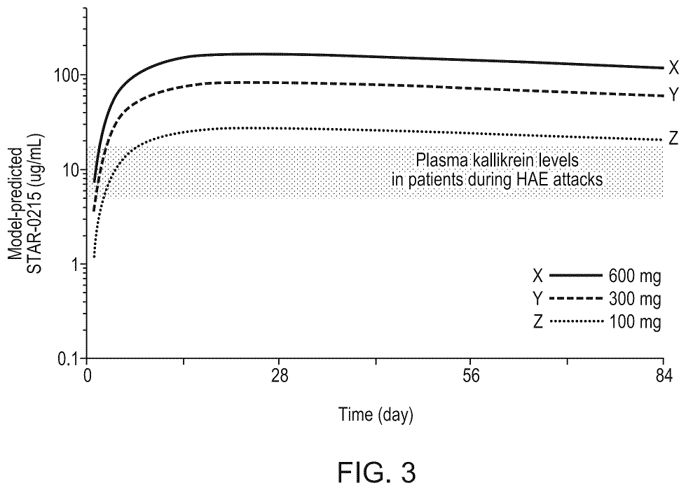

The mPBPK model suggested a prolonged circulating t_{1/2} of STAR-0215 in humans and the simulated profiles (semi-logarithmic scale) are shown in FIG. 3. Simulations are contrasted to target an exposure range of 12 μg/mL (approximately 80 nM), the concentration for adequate inhibition of circulating plasma kallikrein for the resolution of HAE attack in patients. The simulated PK profile of STAR-0215 shows that a single SC administration of STAR-0215 at dose levels of ≥100 mg would achieve exposures that completely inhibit circulating plasma kallikrein and suppress the HAE attacks in patients, for a duration of 3 months or longer. The predicted PK profile suggests that STAR-0215 has a long duration of action for suppressing HAE attacks, making long term prophylactic therapy feasible.

The mPBPK model suggested STAR-0215 would be absorbed rapidly following SC administration and be eliminated more slowly, with a circulating t_{1/2} of approximately 108 days. Based on the projected PK profile, following a single SC administration, a blood concentration of STAR-0215 required to control plasma kallikrein activity in order to prevent HAE attacks, would be achievable within 7 days following a single SC administration. At the dose levels evaluated, the mPBPK model predicts that STAR-0215 would be maintained at blood concentrations that inhibit plasma kallikrein activity inhibition for 3 months or longer.

These data support that a STAR-0215 serum concentration of approximately 80 nM may be sufficient to produce 90% inhibition of plasma kallikrein activity during an HAE attack and may represent the minimal required trough serum concentration required to prevent HAE attacks. This PK model predicts that STAR-0215 need only be administered once every 3 months to provide protection from HAE attacks. This indicates that STAR-0215 could potentially be an improvement on other currently approved prophylactic treatments in terms of efficacy and dosing regimen. In contrast, ORLADEYO® (Berotralstat), a small molecule inhibitor, is limited by its short t_{1/2}, and requires oral administration every day; the SC injected plasma-derived HAEGARDA® (C1 Esterase Inhibitor Subcutaneous (human)) has a relatively short circulating t_{1/2} and has an every 3-4 day dosing schedule; and TAKHZYRO® (lanadelumab), while having as a considerably longer t_{1/2}, however, still requires administration every 2 weeks.

As shown in Table 6 below, a population PK model suggested that, at steady state, about 85% of subjects would experience a Cₘᵢₙ≥25 μg/mL 84 days after a maintenance dose level of ≥600 mg. Accordingly, higher doses, e.g., ≥600 mg of STAR-0215 may, in some cases, lead to longer periods of inhibition of HAE attacks.

TABLE 6

| | | | |
|---|---|---|---|
| | | % Population | % Population |
| Maintenance | Projected Median | with Cₘᵢₙ > | with Cₘᵢₙ > |
| Dose | Cₘᵢₙ Level | 12 μg/mL | 25 μg/mL |
| (mg/kg) | (μg/mL) | (%) | (%) |

At Steady-State Cₘᵢₙ

| | | | |
|---|---|---|---|
| 150 | 12.2 | 51.9 | 5 |
| 300 | 21.6 | 94.4 | 30.8 |
| 450 | 29.3 | 97.3 | 64.2 |
| 600 | 38.8 | 98.7 | 84.9 |

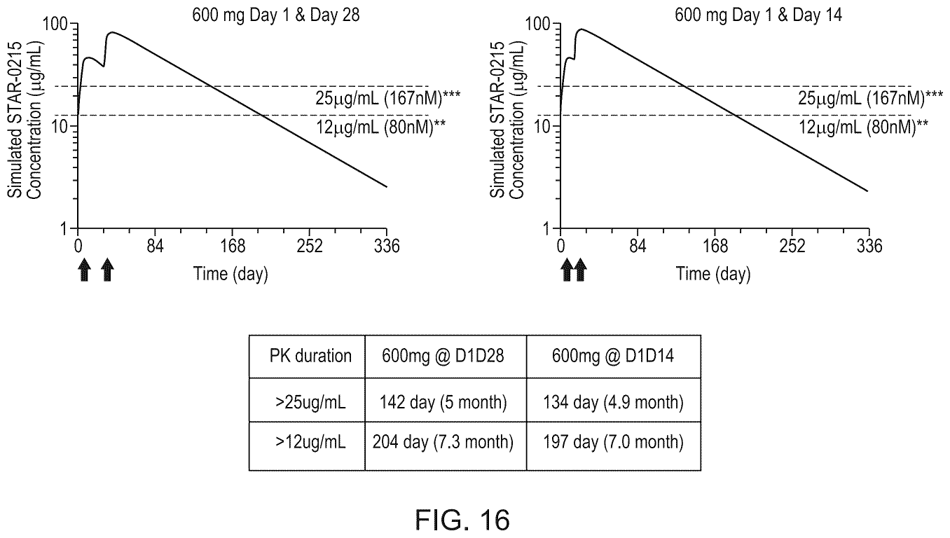

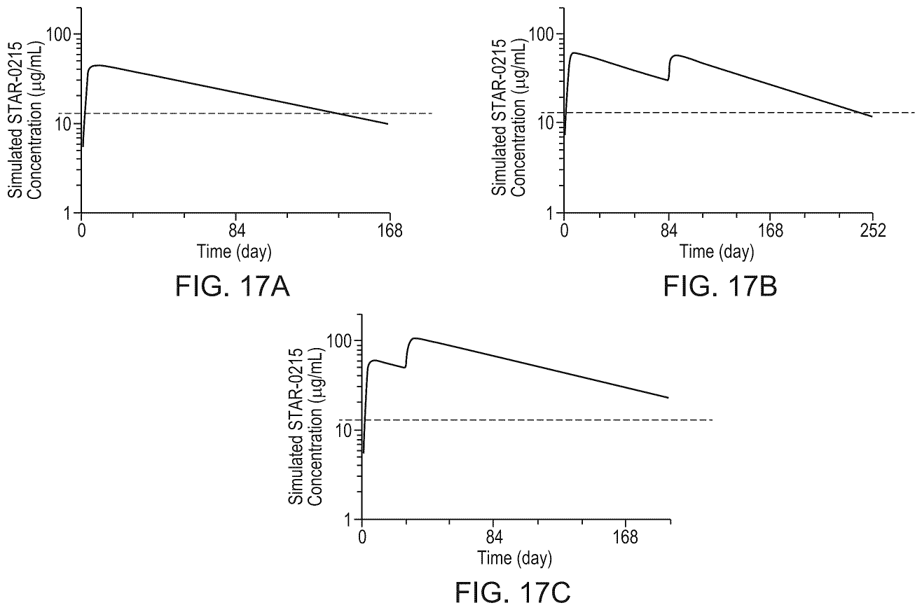

Further population PK modeling was performed to evaluate the pharmacokinetic duration of a 600 mg dose as shown in FIGS. 16 and 17C. The model predicts that a plasma serum concentration of STAR-0215 can be maintained for up to five months by administering 600 mg of STAR-0215 at Day 1 and again at Day 14, or at Day 1 and again at Day 28 (FIG. 16, FIG. 17C). The serum levels of STAR-0215 are predicted to fall below a target Cₘᵢₙ of 12 μg/mL around 7 months, whereas predicted serum levels of STAR-0215 will fall below a target Cₘᵢₙ of 25 μg/mL around 5 months. The Day 1/Day 14 dosing had an increased Cₘₐₓ compared to the Day 1/Day 28 dosing. The predictions show that there is some flexibility around the timing of the second dose, as the data for the Day 1/Day 28 regimen and the Day 1/Day 14 regimen were quite similar with the 25 μg/mL target threshold being exceeded by both regimens for 142 days (5 months) and 134 days (4.9 months) respectively, and the 12 μg/mL target threshold being exceeded by both regimens for 197 days (7 months) and 204 days (7.3 months) respectively. Accordingly, the model predicts that dosing regimens involving a second dose of 600 mg administered anywhere from 2-6 weeks, e.g., 2 weeks, 4 weeks or 6 weeks, after a first 600 mg dose could all achieve pharmacokinetic profiles similar to one another.

Further, as shown in FIG. 17A, population PK modeling predicts that a serum concentration of STAR-0215 can be maintained for at least 3 months after administering a 450 mg dose of STAR-0215 on Day 1, with the 12 μg/mL target threshold being exceeded by the regimen beyond 112 days. Further, as shown in FIG. 17B, population PK modeling predicts that a plasma serum concentration of STAR-0215 above the threshold concentration of 12 μg/mL can be maintained by administering an initial loading dose of 600 mg at Day 1 followed by a 300 mg dose at day 84, suggesting that a dosing regimen of an initial loading dose of 600 mg followed every three months thereafter of 300 mg may be sufficient to suppress HAE attacks.

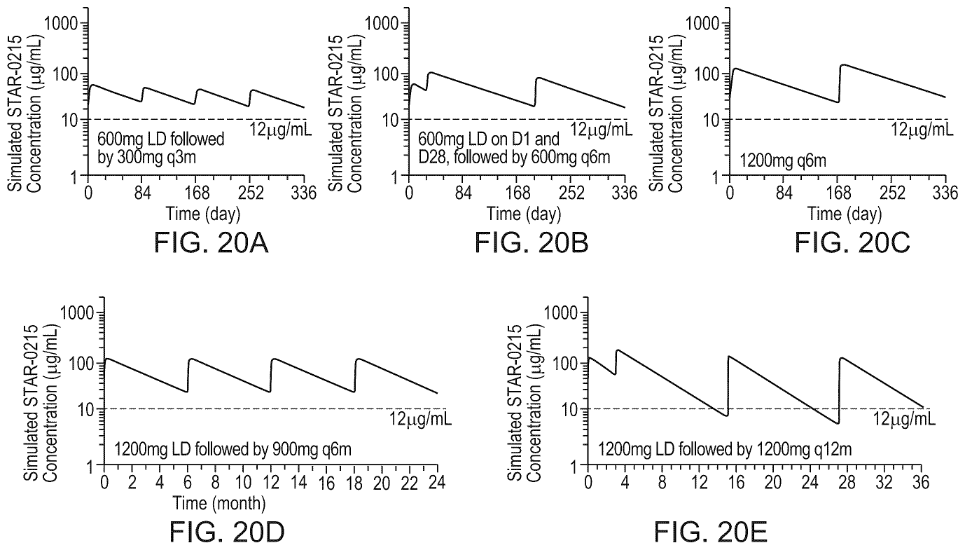

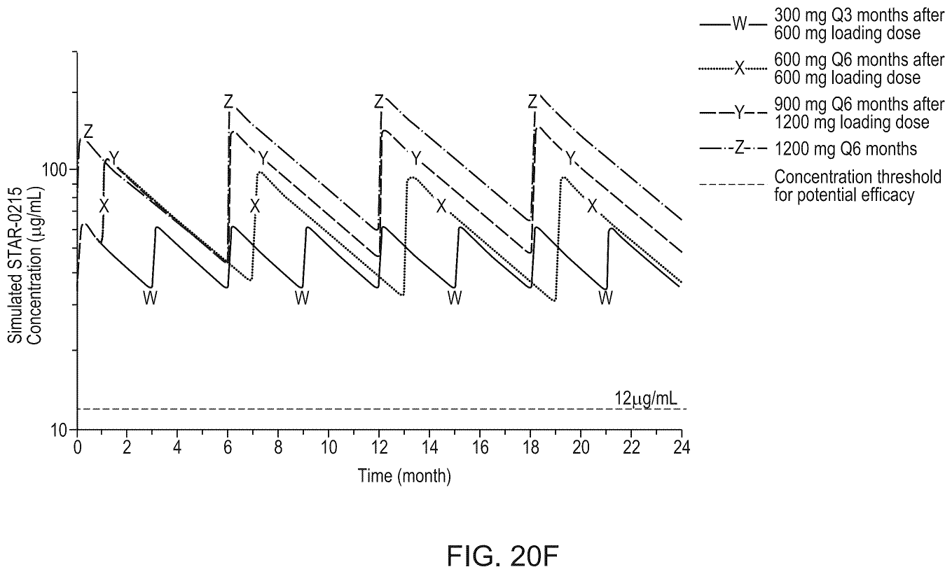

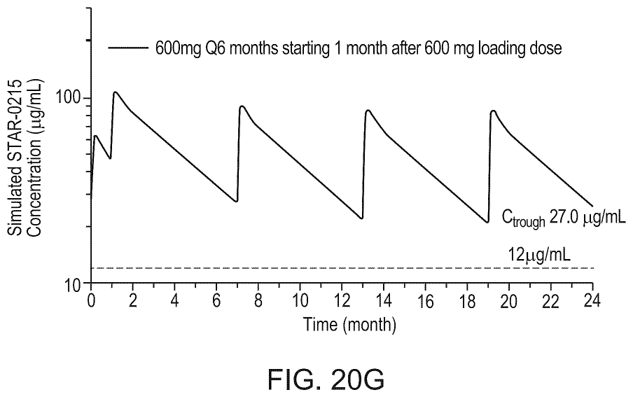

PK modeling was further used to evaluate the pharmacokinetics of various dosing regimens using data obtained from the Phase 1a study in healthy volunteers described below in Example 4. FIG. 20A shows the results of a model of administering a 600 mg loading dose of STAR-0215 administered at Day 1 followed by 300 mg maintenance doses administered every 3 months (84 days) thereafter. The model was run to 336 days past the initial dose. The C_{trough} is 25.1 μg/mL which is the concentration of STAR-0215 at the end of the dosing interval. FIGS. 20B and 20G show the results of the model of administering a 600 mg dose on Day 1, followed by a 600 mg dose on Day 28 and 600 mg again after 6 months. The model was run through 336 days (12 months) past the initial dose in FIG. 20B, and through 24 months past the initial dose in FIG. 20G. The C_{trough} is 27.0 μg/mL which is the concentration of STAR-0215 at the end of the dosing interval. FIG. 20C shows the results of the model administering a 1,200 mg dose on Day 1 followed by 1,200 mg on Day 168. The model was run through 336 days past the initial dose. FIG. 20D shows the results of administering a 1,200 mg loading dose followed by 900 mg maintenance doses every 6 months thereafter. The model was run through 24 months from the initial dose. The model showed that these dosing regimens were sufficient to maintain a simulated STAR-0215 median concentration in excess of 12 μg/mL throughout the duration of the model suggesting that these dosing regimens, when administered to adult human subjects, would be sufficient to suppress HAE attacks. FIG. 20E shows the results of pK modeling of administering 1,200 mg on Day 1 followed by 1,200 mg after 3 months followed by 1,200 mg every 12 months thereafter. The model was run through month 36 after the initial dose. These results demonstrate that this dosing regimen for STAR-0215 can achieve blood levels of STAR-0215 sufficient to suppress HAE attacks in the majority of HAE patients. Accordingly, a range of doses administered every 3-6 months may be effective in HAE attack prevention

### Example 3: Quantitative Systems Pharmacology Modeling (QSP) to Explore Impact of STAR-0215 Dosing Regimens on the Suppression of HAE Attacks

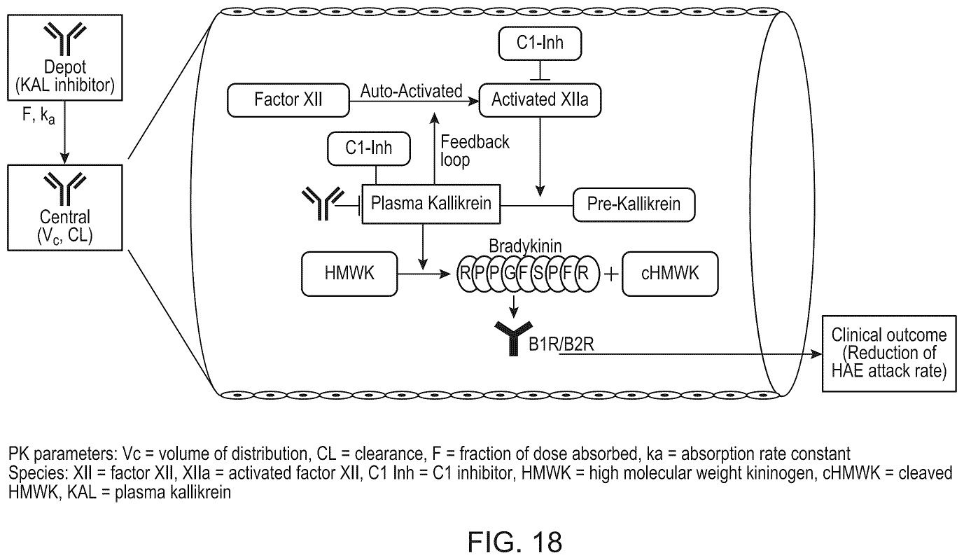

To investigate the potential for and duration of HAE attack suppression by STAR-0215, a simplified, mechanistic, quantitative systems pharmacology (QSP) model was established based on known reaction parameters for the plasma kallikrein-kininogen pathway in the vascular space and adjacent to the endothelial surface. A schematic of the model is shown in FIG. 18.

The QSP model was configured to represent the simplified biological process of the contact system, including production of bradykinin and cleaved HMWK (cHMWK), as well as to determine the likelihood of an HAE attack. This model, when combined with specific pharmacokinetic information regarding adult subjects who receive STAR-0215 can be used to evaluate the binding and inhibition of plasma kallikrein, the inhibition of HMWK cleavage, the generation of bradykinin and cHMWK, and subsequently, to predict the potential of a given dosing regimen to suppress HAE attacks.

The QSP model leveraged “virtual” HAE patients for simulations. The virtual HAE patients' baseline HAE attack frequency was set to be 3 attacks per month (per 28 days), and the timing of the attack event was randomly assigned using a Poisson distribution. Attacks were triggered by an increase in FXII autoactivation and the duration of the auto-activation was set to be 12 hours. An attack event was defined as bradykinin levels reaching a threshold of 20 pM. PK parameter variability was identified from PK data obtained from healthy adult subjects in a Phase 1a trial of STAR-0215 (see Example 4 below). Each simulation was performed on a virtual cohort of HAE patients (n=500 per cohort), and the C1-inhibitor was set to 30% of the healthy control levels.

In the first simulation, a first virtual cohort (n=500) received a dosing regimen of a single subcutaneous loading dose of 600 mg of STAR-0215 on Day 1, followed by subcutaneous maintenance doses of 300 mg of STAR-0215 every 3 months (84 days) thereafter. In a second simulation, a second virtual cohort (n=500) received a dosing regimen of a subcutaneous dose of 600 mg STAR-0215 on Day 1, again on Day 28, and then every 6 months thereafter (every 168 days).

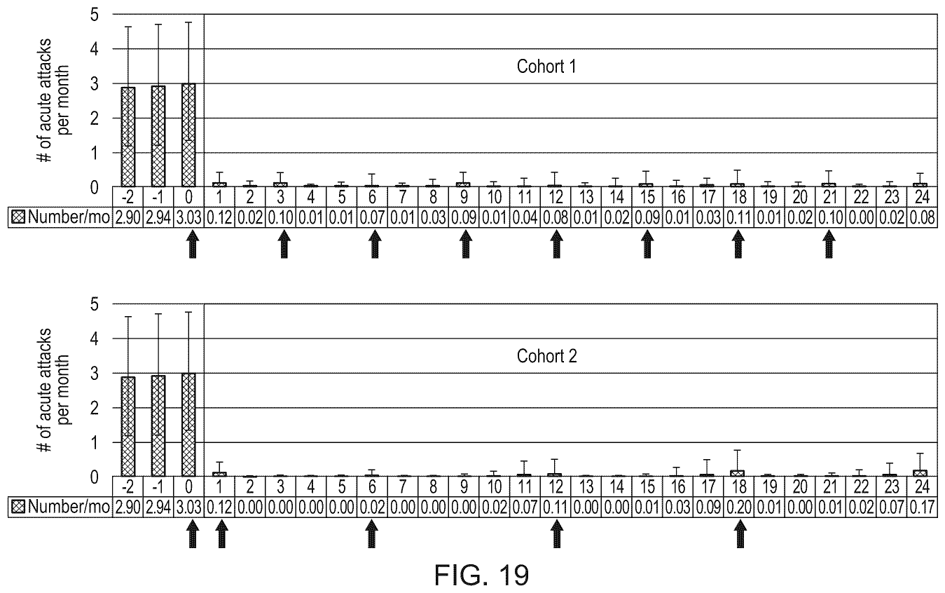

FIG. 19 presents the results of the simulations as bar graphs which show the number of HAE attacks per month for an individual in each of the virtual cohorts. The simulation showed that the baseline attack frequency decreased following STAR-0215 administration. With the dosing regimen of a dose of 600 mg of STAR-0215 on Day 1, followed by subcutaneous maintenance doses of 300 mg of STAR-0215 every 3 months (84 days) thereafter, the attack frequency for the cohort decreased to an average of 0.12 attacks per month in the first month, and maintained an average of <0.25 attacks per month thereafter (Cohort 1 in FIG. 19). Similarly, with the dosing regimen of a subcutaneous dose of 600 mg STAR-0215 on Day 1, again on Day 28, and then every 6 months thereafter (every 168 days), the attack frequency decreased to an average of 0.12 attacks per month in the first month, and maintained an average of <0.25 attacks per month thereafter (Cohort 2 in FIG. 19). The model estimated a ≥90% reduction in monthly HAE attack rate starting at the 1st month of treatment for both dosing regimens. The model also estimated an average of 80% of treated HAE patients to be attack-free during the first 6 months of treatment, for both dosing regimens.

Accordingly, the results from the QSP model showed that a 600 mg loading dose, followed by 300 mg maintenance dose every 3 months would provide effective HAE attack suppression. The option for every 6-month dosing was also evaluated and the results showed that an initial dose of 600 mg followed one month later by a 600 mg dose with 600 mg doses following every 6 months thereafter would also adequately suppress HAE attacks.

### Example 4: Phase 1a Clinical Study

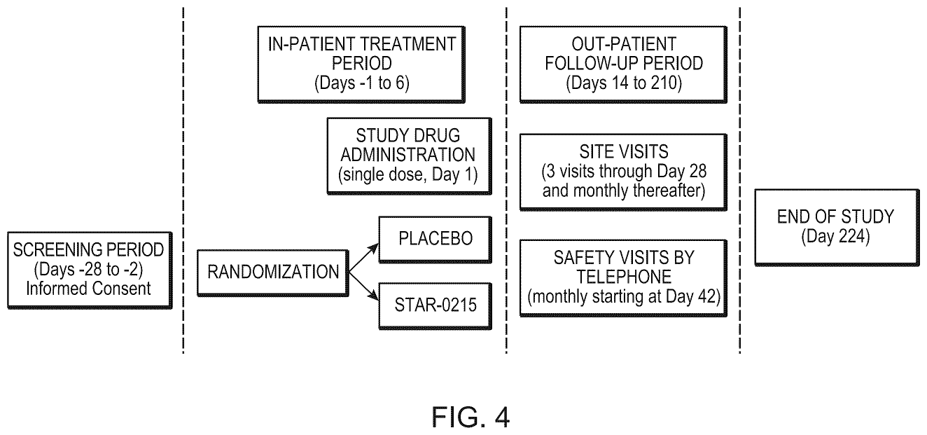

A first-in-human, Phase 1a, Randomized, Double-Blind, Placebo Controlled, Single Ascending Dose Study was undertaken to assess the safety, tolerability, pharmacokinetics, pharmacodynamics, and immunogenicity of STAR-0215 in healthy adult subjects. A schematic diagram showing the stages of the Phase I study design is provided in FIG. 4. The primary objective of the study was to assess the safety and tolerability of escalating single subcutaneous administrations of STAR-0215 in healthy adult subjects. Secondary objectives of the study were to characterize the pharmacokinetics (PK) of escalating single subcutaneous administrations of STAR-0215 in healthy adult subjects, to characterize the pharmacodynamics (PD) of escalating single subcutaneous administrations of STAR-0215 in healthy adult subjects, and to assess the immunogenicity of escalating single subcutaneous administrations of STAR-0215 in healthy adult subjects.

Primary Endpoints of the study were to observe adverse events (AEs) and serious adverse events (SAEs), obtain clinical laboratory results, 12-lead ECG and vital signs. The secondary endpoint was to obtain serum concentrations of STAR-0215 and the derived pharmacokinetic parameters.

An exploratory objective of the study was to evaluate exploratory laboratory biomarkers. The exploratory endpoint was to obtain an orthogonal measure of plasma kallikrein activity.

The study design employed a standard approach for differentiation between the safety profiles of an active and placebo treatment when administered to subjects. The safety measures employed in this protocol were standard measures routinely used for the evaluation of the safety and tolerability of an investigational product.

### Methodology and Study Design

Healthy adult subjects aged 18 to 60 were eligible to participate in the study. Eligible subjects were randomized 3:1 to receive STAR-0215 or placebo in cohorts of 8 subjects each. In each cohort, subjects received 1 dose of study drug (STAR-0215, supplied as a sterile solution for subcutaneous administration in a single-use vial at a concentration of 150 mg/ml) or placebo (sterile solution of 0.9% saline for subcutaneous administration), administered subcutaneously by bolus injection into the abdomen on day 0. There were 3 cohorts with dose levels of 100, 300, and 600 mg, and with a potential for up to 3 additional cohorts to be added. Two additional cohorts were added after the commencement of the study, one receiving 1,200 mg by bolus subcutaneous administration and the other receiving 600 mg by IV administration. Each of these additional cohorts included 8 subjects randomized 3:1 to receive STAR-0215 or placebo.

Properly consented, screened, and eligible subjects were admitted to the clinic on Day-1 and remained in the clinic through the assessments performed 120 hours after study drug administration. Subjects were discharged from the clinic on Day 6 after all required study procedures were completed and after being cleared by the Investigator or designee. Subjects returned to the study site for 10 follow-up visits through 224 days after study drug administration (3 visits through the first month after study drug administration (Day 28) and 7 monthly visits thereafter). Safety visits were conducted by telephone between the monthly visits. In each cohort, a sentinel group of 2 subjects was randomized and dosed on Day 1 (1 active and 1 placebo). The remaining six subjects in each cohort were dosed after no clinically significant safety or tolerability issues were noted in the sentinel group within the first 24 hours after dosing. Available data from an individual cohort through 14 days after the administration of STAR-0215 was reviewed before dose escalation. A 14-day safety review period was employed based on the SC absorption profile of STAR-0215 and an anticipated Tₘₐₓ of 2 to 8 days.

The dose regimen was as shown in Table 7:

TABLE 7

| | STAR-0215 Dose Level | # Active | # Number Placebo |
|---|---|---|---|
| Cohort | (mg) | Subjects | Subjects |
| 1 | 100 SC | 7 | 2 |
| 2 | 300 SC | 6 | 2 |
| 3 | 600 SC | 6 | 2 |
| 4 | 1,200 SC | 6 | 2 |
| 5 | 600 IV bolus | 6 | 2 |

The duration of study participation for each subject was up to 252 days including screening.

Safety assessments included adverse events (AEs), vital signs, physical examinations, electrocardiograms, and clinical laboratory results, including chemistry, hematology, coagulation, and urinalysis. For pharmacokinetic analysis, blood samples were collected to measure the serum concentration of STAR-0215 before and after study drug administration. For pharmacodynamic analysis, samples were collected to measure the plasma levels of cleaved high-molecular-weight kininogen (cHMWK; measure of plasma kallikrein activity). For immunogenicity assessments, blood samples were collected to assess the formation of STAR-0215 anti-drug antibodies in serum before and after study drug administration.

41 healthy subjects were randomized in this study. This was considered adequate to characterize the safety, tolerability, PK, PD, and immunogenicity of STAR-0215. Data was summarized using descriptive statistics (mean, median, standard deviation, minimum and maximum) or frequency counts and percentages, as appropriate. Results are presented by dose of STAR-0215 vs combined placebo.

### Results

Pharmacodynamic analysis: Plasma samples obtained from each cohort (placebo, 100 mg, 300 mg, 600 mg, and 1,200 mg SC, and 600 IV) were evaluated to determine the levels of plasma kallikrein inhibition resulting from administration of STAR-0215. Because study volunteers were healthy and therefore had low, non-pathologic levels of plasma kallikrein and consequently low levels of cHMWK, the plasma was activated ex vivo by FXIIa leading to increased plasma kallikrein activity and therefore increased cleavage of HMWK to cHMWK. It was hypothesized that STAR-0215 would inhibit pKal activity and therefore lower levels of cHMWK would be observed. Western blot analysis was performed to determine the levels of both HMWK and cHMWK in the plasma samples from each cohort.

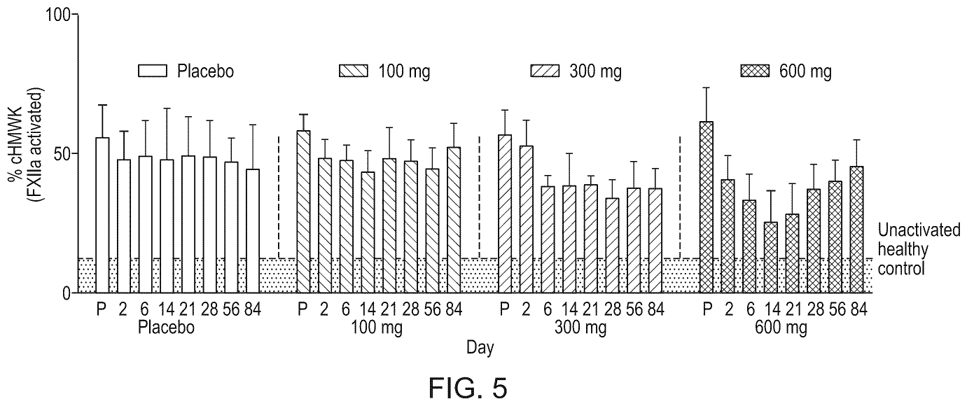

As shown in FIG. 5, STAR-0215 demonstrated sustained engagement with the target, pKal leading to functional pKal inhibition. There was a significant reduction in levels of HMWK cleavage at least from day 2 through day 84 for the 600 mg cohort and from at least day 6 through day 84 in the 300 mg cohort as compared to the predose levels.

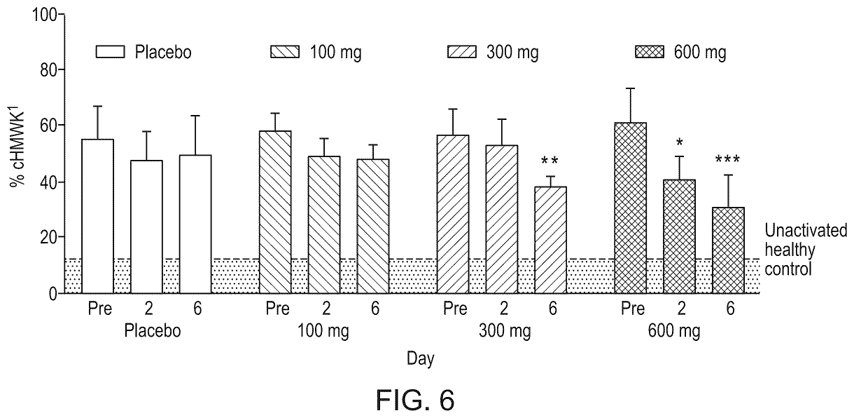

As shown in FIG. 6, STAR-0215's engagement of pKal was rapid and dose related, providing statistically significant decreases in cHMWK levels by day 6 with a 300 mg dose and by day 2 with a 600 mg dose.

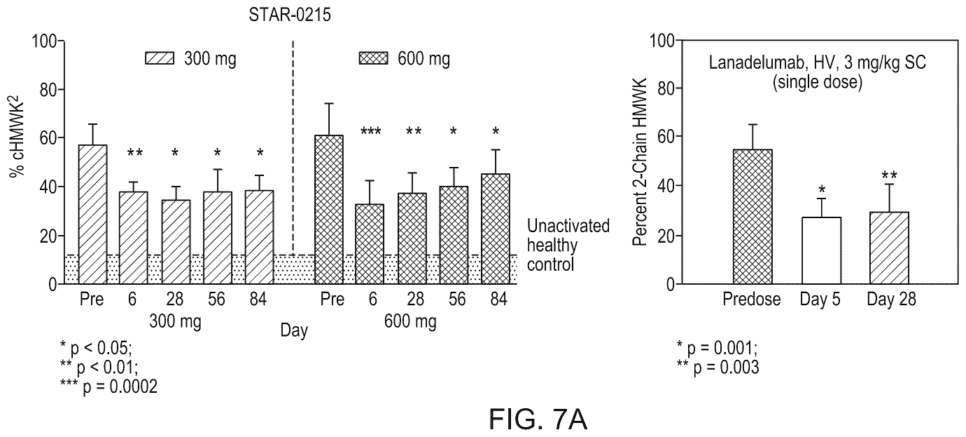

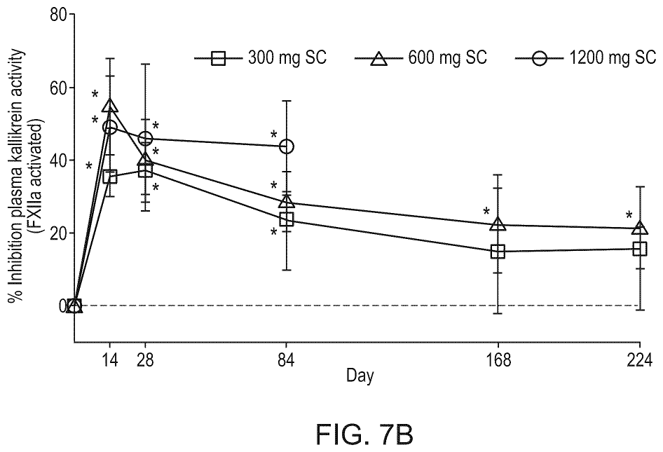

As shown in FIG. 7A, STAR-0215 achieved sustained inhibition of ex vivo FXIIa activated pKal as determined by cHMWK through day 84 for the 300 mg dose and the 600 mg dose as compared to predose (P) levels. These levels of cHMWK are similar to levels of pKal activation observed by Chyung et al. (Ann Allergy Asthma Immunol, 2014, 113:460-466) with respect to lanadelumab, another commercially available anti-pKal antibody. Data for the 1,200 mg SC cohort through day 84 is presented in FIG. 7B, which shows that STAR-0215 also achieved sustained inhibition of ex vivo FXIIa activated pKal as determined by cHMWK. FIG. 7B, which also includes pKal inhibition for the 600 mg cohort, shows that inhibition of plasma kallikrein activity was sustained to 224 days after a single dose of 600 mg of STAR-0215, suggesting that the potential clinical efficacy to reduce/inhibit HAE attacks for the 600 mg and 1,200 mg doses for a sustained period of time.

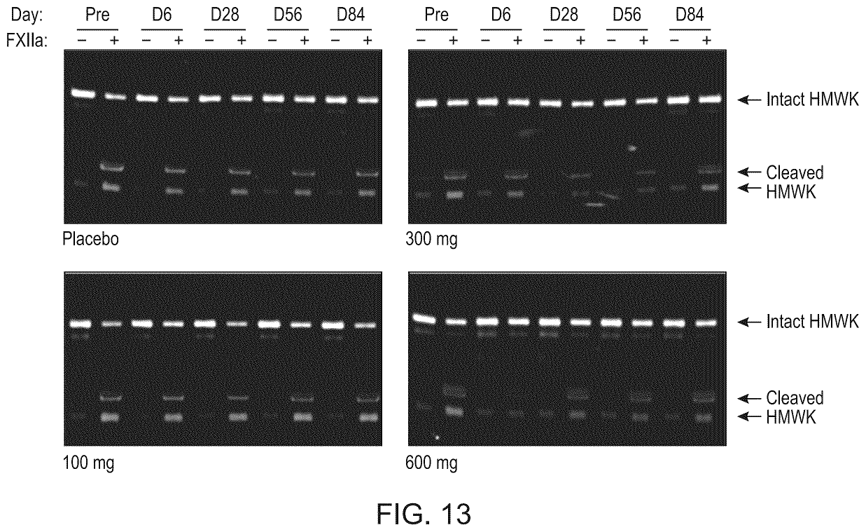

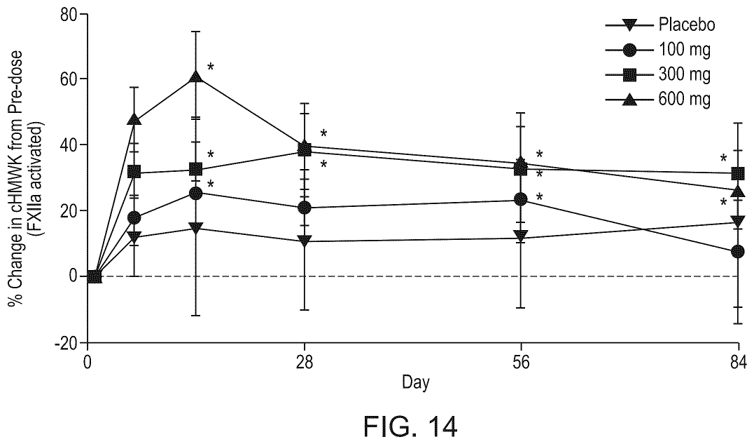

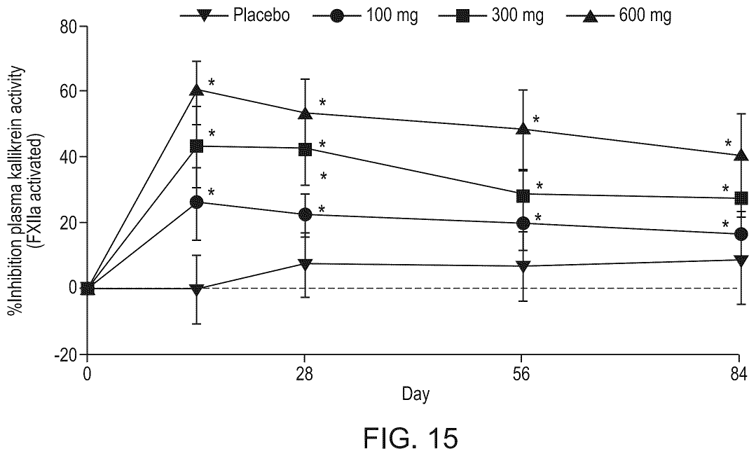

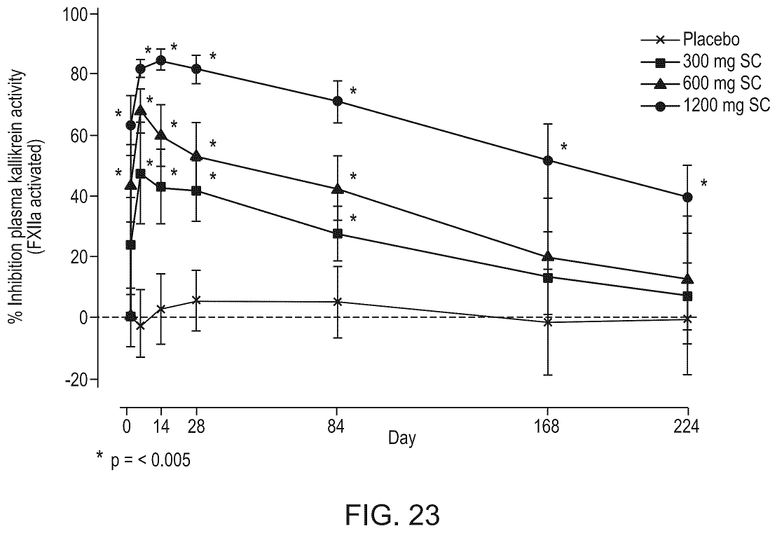

Pharmacodynamic analysis also revealed that suppression of cHMWK to levels consistent with robust plasma kallikrein inhibition by STAR-0215 was achieved through Day 84 as observed in an ex vivo FXIIa-induced cleavage of plasma HMWK Western blot assay, the results of which are shown in FIGS. 13 and 14, as well as in an orthogonal chromogenic reporter-substrate (PFR-pNA) assay, results of which are shown in FIG. 15. As shown in FIG. 23, a statistically significant (p<0.005) inhibition of ex vivo FXIIa-activated plasma kallikrein activity was observed through day 84 after single doses of 300 mg and 600 mg subcutaneous administration, and through day 224 after a single dose of 1,200 mg subcutaneous administration. This data suggests the potential for a 1,200 mg dose administered infrequently, e.g., at least every 6 months or more, such as every 7 months, every 8 months, or every 9 months or more, while still inhibiting pKal activity.

For the ex vivo FXIIa-induced cleavage of plasma HMWK Western blot assay, sodium citrate plasma samples from subjects in each cohort were incubated ex vivo in the presence or absence of 5 nM FXIIa to activate plasma kallikrein. (Samples without added exogenous FXIIa were used as controls and are seen in FIG. 13 as rows marked “—”). The samples were protease-inhibited and prepared for loading on a 4-20% tris-glycine polyacrylamide gel, followed by transfer to a PVDF membrane. The separated HMWK and cHMWK bands were visualized with a goat anti-human kininogen antibody and IR dye labeled donkey anti-goat IgG secondary antibody. Target band signal intensities were captured with an Odyssey Imager and percentage of cHMWK in the sample relative to total HMWK+cHMWK was calculated.

As shown in FIG. 13, the presence of STAR-0215 inhibited plasma kallikrein activity and thus formation of the cHMWK relative to predose activated samples. The bands of cleaved HMWK were most intense for the 100 mg cohort, and least intense for the 600 mg cohort for all time points, showing that the 600 mg dose resulted in lower cleavage from higher pKal inhibition. The results for the 300 mg cohort were similar to those for the 600 mg cohort.

As shown in FIG. 14, the percent change in cHMWK relative to total HMWK+cHMWK in the 300 mg and 600 mg cohorts, as measured by the signal intensities, was statistically significant through day 84, indicating significant inhibition of cHMWK at all post dose timepoints for these two cohorts, whereas the percent change for the 100 mg cohort was not statistically significant beyond Day 56 (p<0.05).

FIG. 15 shows the results of an orthogonal chromogenic reporter-substrate (PFR-pNA) assay to determine percent inhibition of plasma kallikrein activity when exogenously activated by Factor XIIa. Sodium citrate plasma samples were incubated ex vivo in the presence or absence of 10 nM FXIIa to activate plasma kallikrein, followed by addition of Pro-Phe-Arg-p-nitroaniline (PFR-pNA) chromogenic substrate. The activity of plasma kallikrein was measured by the increase in absorbance due to cleavage of substrate to p-nitroaniline. The presence of STAR-0215 inhibited plasma kallikrein activity and thus formation of the colorimetric product p-nitroaniline relative to predose FXIIa activated samples. As shown in FIG. 15, for each of the 100 mg, 300 mg, and 600 mg cohorts, the results were statistically significant (p<0.05) for each time point. This assay demonstrates a dose-responsive, robust, and long-lasting inhibition of plasma kallikrein activity after a single dose of STAR-0215. As shown in FIG. 23, a statistically significant (p<0.005) inhibition of ex vivo FXIIa-activated plasma kallikrein activity was observed through day 84 after single doses of 300 mg and 600 mg subcutaneous administration, and through day 224 after a single dose of 1,200 mg subcutaneous administration.

Overall, the plasma kallikrein inhibition effects of STAR-0215 were found to be rapid, sustained, and consistent with or superior to those achieved by lanadelumab.

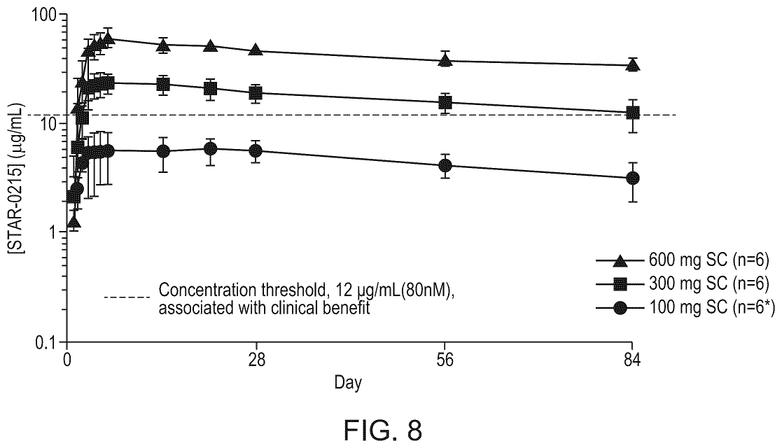

Pharmacokinetic analysis: The levels of STAR-0215 in the plasma of patients from each cohort were determined over the study period. The results, as shown in FIG. 8, indicate that subcutaneous dosing of 100 mg, 300 mg and 600 mg of STAR-0215 resulted in a rapid attainment of Cₘₐₓ followed by sustained concentrations of STAR-0215 through day 84 in the 100 mg, and 300 mg, and 600 mg cohorts. Accordingly, as demonstrated in FIG. 8, the serum concentrations of STAR-0215 are proportional to the dose with an estimated half-life of 117 days. Both the 300 mg and 600 mg doses maintained or exceeded a threshold concentration of 12 μg/mL (80 nM) believed to be associated with clinical benefit through day 84, suggesting that these doses may be effective to inhibit pKal when dosed at 3 month intervals. The elimination phase for the antibody is shown to be long.

A summary of pharmacokinetic parameters of STAR-0215 based on a day 224 final analysis in the 100, 300, and 600 mg SC cohorts, and an interim analysis for the ongoing 1,200 mg SC and 600 mg IV cohorts (Day 112 and Day 84 respectively) is provided in Table 8 below. (The data for the 100 mg cohort is presented twice, once for the total group of study patients based on an n=7, and again based on an n=6* where one patient who received a partial dose was excluded.)

TABLE 8

| | | | | | Cₐᵥₑ | AUC₀₋₈₄ | | |
|---|---|---|---|---|---|---|---|---|
| | T_{1/2} | T_{12 μg/ml} | Tₘₐₓ | Cₘₐₓ | Day | (days* | CL/F | Cₗₐₛₜ |
| Dose Group | (days) | (hrs) | (days) | (μg/ml) | 84 | μg/ml) | (mL/h) | (μg/ml) |

100 mg, N = 7

| | | | | | | | | |
|---|---|---|---|---|---|---|---|---|
| Mean | 69 | n/a | 13 | 6.4 | 2.7 | 354 | 5.4 | 3.0 |
| SD | 21.3 | | 10 | 2.1 | 1.7 | 136 | 1.99 | 1.3 |

100 mg, N = 6*

| | | | | | | |
|---|---|---|---|---|---|---|
| Mean | 83 | n/a | 13 | 6.57 | 385 | 2.60 |
| SD | 28 | | 11 | 2.23 | 118 | 1.08 |

300 mg, N = 6

| | | | | | | | | |
|---|---|---|---|---|---|---|---|---|
| Mean | 83 | 25 | 8 | 24.9 | 12.5 | 1445 | 4.8 | 12.5 |
| SD | 12.8 | | 4 | 6.1 | 4.47 | 314 | 1.87 | 4.5 |

600 mg, N = 6

| | | | | | | | | |
|---|---|---|---|---|---|---|---|---|
| Mean | 94 | 11 | 6 | 64.0 | 35.7 | 3762 | 3.7 | 35.7 |
| SD | 20.9 | | 3 | 13.4 | 5.09 | 581 | 0.67 | 5.1 |

1,200 mg SC, N = 6

| | | | | | | | |
|---|---|---|---|---|---|---|---|
| Mean | 127 | 7 | 4.8 | 154.2 | 72.7 | 8100 | n/a |
| SD | 36.7 | | 0.41 | 40.72 | 16.18 | 1761 | |

600 mg IV, N = 6

| | | | | | | | |
|---|---|---|---|---|---|---|---|
| Mean | 109 | <0.1 | 0.13 | 203.2 | 55.5 | 6658 | n/a |
| SD | 35.8 | | 0.10 | 27.17 | 11.24 | 1077 | |

Abbreviations: t_{1/2}, half-life; t_{12 μg/mL}, time to reach concentration threshold; Tₘₐₓ, time to reach peak drug concentration; Cₘₐₓ, peak drug concentration; Cₐᵥₑ, average concentration on day 84; AUC_{0-84 day}, area under concentration-time curve from time 0 to 84 days; CL/F,
clearance; Cₗₐₛₜ is the last concentration measured at the end of the study period.

The pharmacokinetic analysis shows that STAR-0215 demonstrated a dose-dependent PK, rapid absorption with an average Tₘₐₓ between 4.8-13 days for the SC cohorts, and an estimated t_{1/2} up to 127 days. Dose-related increases in Cₘₐₓ and AUC were observed. The STAR-0215 concentration exceeded 12 μg/mL (80 nM) (threshold for potential efficacy) approximately 11 hours after administration. At Day 84, mean concentrations remained above 12 μg/mL (80 nM) after a single 300 mg SC dose and 3 times greater than 80 nM after 600 mg SC dosing suggesting that 300 mg and 600 mg doses have potential for infrequent dosing, e.g., every three months. The data for the 1,200 mg SC and 600 mg IV cohorts suggest infrequent dosing at even longer intervals, e.g., at least every 6 months or more.

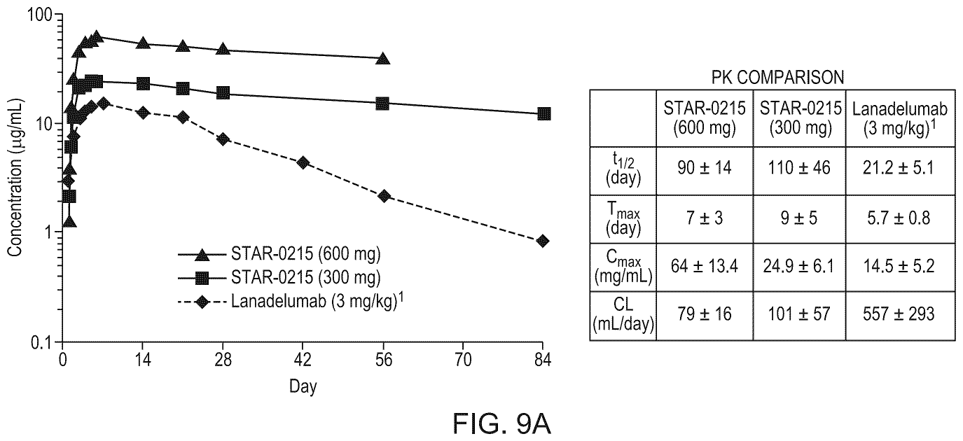

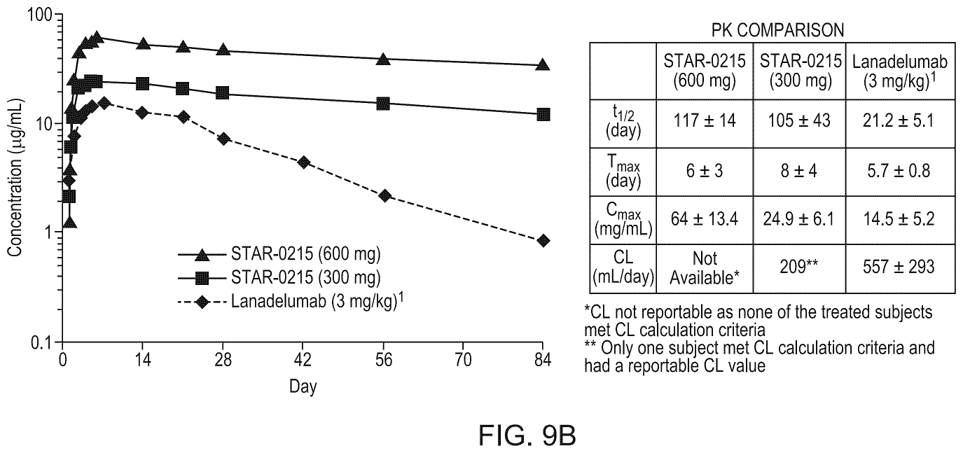

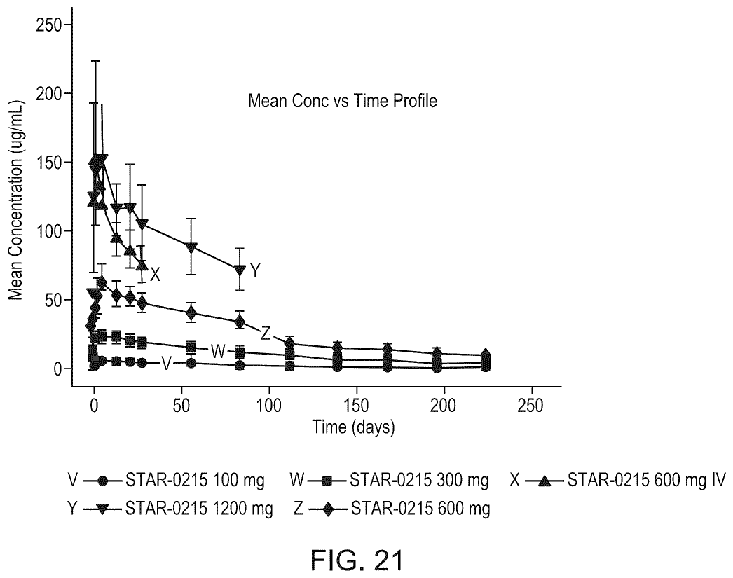

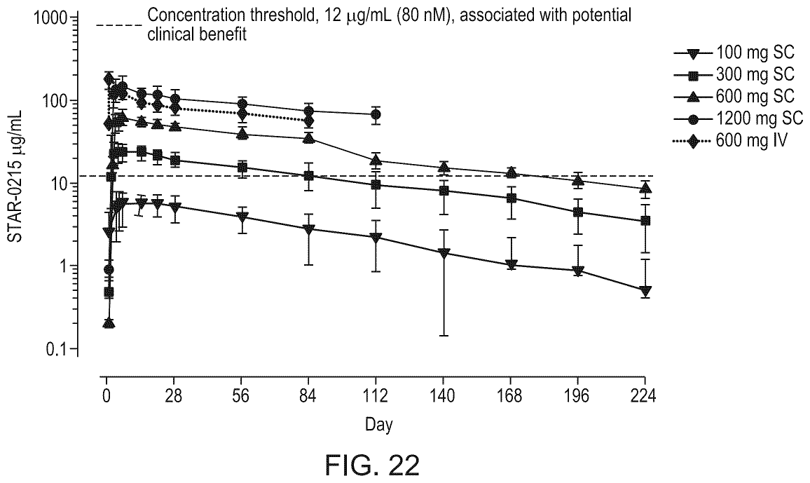

The serum levels of STAR-0215 in the 300 mg and 600 mg were compared to those of lanadelumab as reported by Chyung et al. (Ann Allergy Asthma Immunol, 2014, 113:460-466) as shown in FIGS. 9A-B. The plasma concentration of lanadelumab dropped significantly after 28 days. In contrast, the serum concentration of STAR-0215 remained steady for the 300 mg and 600 mg dose cohorts through day 84. The estimated half-life of STAR-0215 at the 600 mg dose based on FIG. 9A was 90±14 and for 300 mg was 110±46 compared to 21.2±5.1 for lanadelumab. The clearance rate (CL) for STAR-0215 compared to lanadelumab was markedly lower as well. When the day 84 data for the 600 mg cohort was included, as shown in FIG. 9B, the half-life of STAR-0215 at the 600 mg dose was 117±14 days compared to compared to 21.2±5.1 for lanadelumab. CL was not available for the 600 mg cohort as shown in FIG. 9B as none of the cohort members met the calculation criteria (*), and for the 300 mg cohort as shown in FIG. 9B, only one subject met the CL calculation criteria and had a reportable CL (**). (The difference in PK values provided in FIGS. 9A and 9B for the 300 mg cohort may be attributed to the fact that the number of subjects in the cohort changed over time). For the 1,200 mg SC cohort, data available at 84 days showed the mean concentration of STAR-0215 was 72.7 μg/mL (FIG. 21), and at day 112 was 66.6 μg/mL (FIG. 22), both time points being well above the 12 μg/mL threshold believed necessary for HAE inhibition. At 28 days, the mean concentration of STAR-0215 for the 600 mg i.v. cohort was 75.4 μg/mL (FIG. 21), and at day 84 was 55.5 μg/mL (FIG. 22), well above the 12 μg/mL threshold believed necessary for HAE inhibition.

These pharmacokinetic data demonstrate rapid increase in concentration of STAR-0215 in the blood after both subcutaneous and intravenous administration and dose-related increases in concentrations and parallel elimination phases. That these doses reached or exceeded the therapeutic threshold quickly (≤1 day) implies potential for rapid-onset clinical benefit in preventing HAE, as well as sustained clinical benefit for ≥3 months at doses >100 mg SC as for each cohort (with the exception of the 100 mg SC cohort) mean concentrations remained at 12 μg/mL for ≥84 days (FIG. 22). These data suggest that STAR-0215 has potential to be a best in class molecule for treating HAE given the demonstrated long half-life and sustained circulating concentrations to day 84 for the 300 mg cohort, which will permit dosing about every three months, and about every six months based on the results of the 600 mg cohort through day 168 (FIG. 22) due to demonstrated sustained levels of STAR-0215 above the therapeutic threshold in the 300 mg and 600 mg SC cohorts. Extrapolating the results from the 600 mg IV (Day 84) and 1,200 mg SC cohorts (Day 112) as shown in FIG. 22 suggest that similarly long intervals between doses, e.g. at least every six months or more, may be possible due to demonstrated sustained levels of STAR-0215 above the therapeutic threshold.

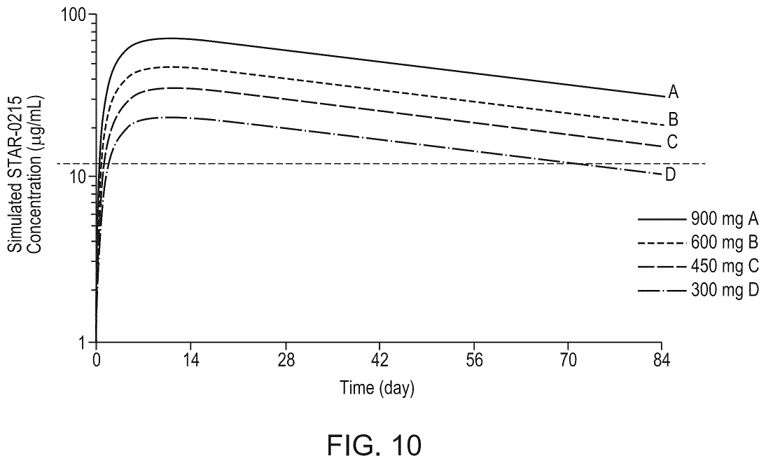

Based on the PK data obtained from the Phase 1a study, an updated PK model was created that suggests a single dose of ≥300-450 mg could achieve the target plasma level of STAR-0215 for a duration of 3 months. The results of the PK modeling are shown in FIG. 10.

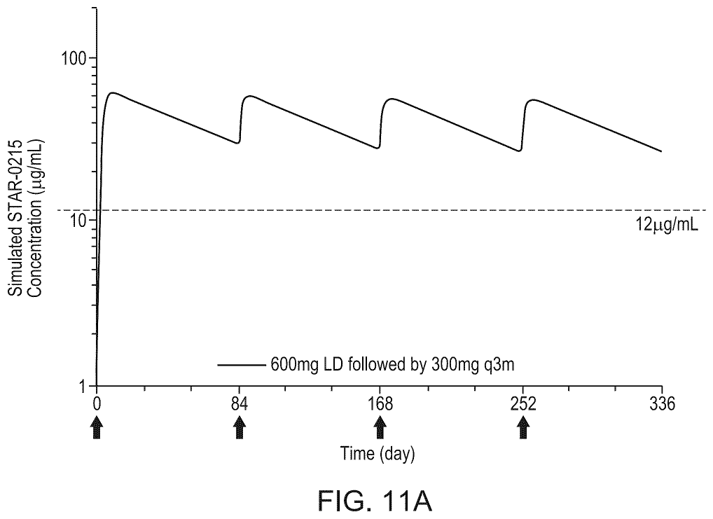

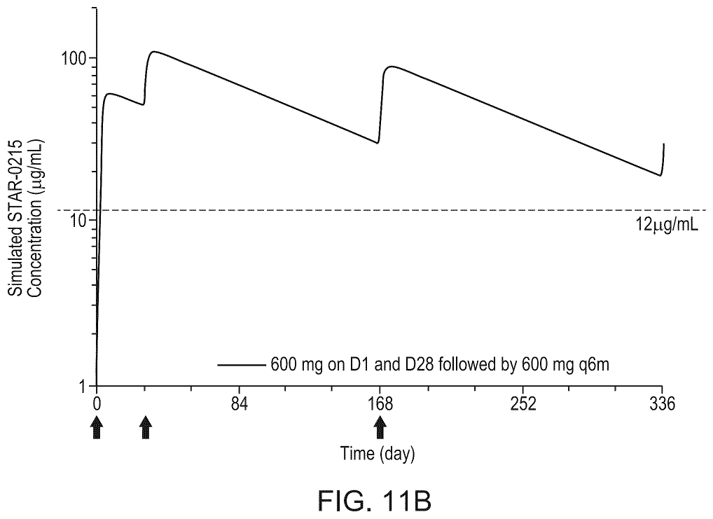

Based on the results from the PK data and the updated modeling, a simulated human PK profile was generated as shown in FIG. 11A. A loading dose of 600 mg at day 0 followed by 300 mg maintenance doses in 84 day (3 month) intervals thereafter was shown by the model to be sufficient to maintain a serum concentration of STAR-0215 in excess of the threshold concentration of 80 nM (12 μg/mL) believed necessary for pKal inhibition sufficient to prevent HAE attacks. The steady state concentration contemplated to be achieved by the model was 27 μg/mL, which is believed to be sufficient to confer HAE attack prevention.

A second simulated human PK profile was generated as shown in FIG. 11B. An initial dose of 600 mg at day 1 followed by a 600 mg dose at day 28 and every 6 months thereafter (168 days) was shown by the model to be sufficient to maintain a serum concentration of STAR-0215 in excess of the threshold concentration of 80 nM (12 μg/mL) believed necessary for pKal inhibition sufficient to prevent HAE attacks. The steady state concentration contemplated to be achieved by the model is 16 μg/mL. This simulation was extended for 24 months as shown in FIG. 20G; the simulation showed that a concentration above 27.0 μg/mL (C_{trough}) could be maintained by administration every 6 months. It is contemplated that this level of STAR-0215 could confer HAE attack prevention. Accordingly, based on these simulations dosing every 3 months or every 6 months is contemplated to confer clinical benefit.

80 nM (12 μg/mL) was conservatively selected to be the target level for STAR-0215 based on data from other antibody and protein inhibitors of pKal. Ecallantide, a recombinant protein comprised of 60 amino acids, is a human plasma kallikrein inhibitor, approved for the symptomatic treatment of acute HAE attacks. It has been shown that with a dose level of 30 mg, a subcutaneous administration of ecallantide resulted in adequate plasma kallikrein inhibition for the symptomatic treatment of acute HAE attacks (See, e.g., EMA Assessment Report for Kalbitor, 2011). This evidence suggested the Cₘₐₓ of ecallantide (586 ng/mL [˜80 nM]) after a subcutaneous administration at 30 mg is a consequential target for adequate inhibition of circulating plasma kallikrein during HAE attacks. The correlation between the clinical effect (resolution of pKal-triggered HAE attack) and 80 nM ecallantide concentration implies that the circulating level of pKal is ˜80 nM during an acute attack.

In addition, lanadelumab, a monoclonal antibody plasma kallikrein inhibitor, is approved for prophylactic treatment for HAE. In a clinical study where the dose regimens of 150 mg q4 week, 300 mg q4 week and 300 mg q2 week were tested, it was reported that the effect of HAE attack suppression was similar between the dose regimens of 300 mg q2 week and 300 mg q4 week (and less effective for 150 mg q4 week) (Wang et al., 2020, Clin Transl Sci, 13:1208-1216). This evidence suggests that the steady state maintenance concentration of lanadelumab at the dose level of 300 mg q4 week (8.77 μg/mL [˜60 nM]) is needed for effective HAE attack suppression, and that of 150 q4 week (4.81 μg/mL [˜33 nM]) may not be sufficient. As for lanadelumab, these data suggest that the concentration of pKal during an attack is ˜60 nM.

Taken together, the data suggest that a minimum circulating concentration of a pKal inhibitor to induce clinical benefit in HAE-C1INH is 60-80 nM. Therefore, 80 nM was conservatively selected as the target serum concentration level for STAR-0215.

The study data from all cohorts showed that STAR-0215 was safe and well tolerated at the doses administered based on administration to 31 subjects receiving STAR-0215 and 10 receiving placebo. Related treatment emergent adverse events (TEAEs) were seen in 11 subjects (STAR-0215 n=10; placebo n=1), were mild (Grade 1) in severity, and resolved without intervention. Most common related treatment emergent adverse events occurring in >2 STAR-0215 subjects include injection site reactions of erythema (22.6%), pruritus (12.9%), and swelling (12.9%). One subject experienced (100 mg cohort) unexpected weight gain, while another subject (placebo group) experienced headache, and another in the 1,200 mg SC experienced injection site pain. There were no serious adverse events or discontinuations due to an adverse event. No clinically significant changes in vital signs, ECG parameters, or laboratory assessments, including activated partial thromboplastin time (aPTT), were observed. With respect to immunogenicity, treatment-emergent anti-drug antibodies (ADAs) were detected in six subjects. Table 9 summarizes the Treatment-Emergent Adverse Events. The results indicate that STAR-0215 was well-tolerated and had a favorable safety profile, with low risk for injection site pain.

TABLE 9

| | | | | | STAR- | | | | |
|---|---|---|---|---|---|---|---|---|---|
| | STAR- | STAR- | STAR- | | 0215 | STAR- | Combined | | |
| | 0215 | 0215 | 0215 | | 1,200 mg | 0215 | STAR- | | Combined |
| | 100 mg SC | 300 mg SC | 600 mg SC | | SC | 600 mg IV | 0215 | | Placebo |
| | (N = 7) | (N = 6) | (N = 6) | | (N = 6) | (N = 6) | (N = 31) | | (N = 10) |
| Any | 5 (71.4) | 3 (50.0) | 6 | (100.0) | 4 (66.7) | 3 (50.0) | 21 | (67.7) | 5 (50.0) |
| TEAE, | | | | | | | | | |
| n (%) | | | | | | | | | |
| Related | 1 (14.3) | 2 (33.3) | 4 | (66.7) | 3 (50.0) | 0 | 10 | (32.3) | 1 (10.0) |
| TEAE, | | | | | | | | | |
| n (%) | | | | | | | | | |
| Injection | 0 | 2 (33.3) | 3 | (50.0) | 2 (33.3) | 0 | 7 | (22.6) | 0 |
| Site | | | | | | | | | |
| Erythema | | | | | | | | | |
| n (%) | | | | | | | | | |
| Injection | 0 | 1 (16.7) | 2 | (33.3) | 1 (16.7) | 0 | 4 | (12.9) | 0 |
| Site | | | | | | | | | |
| Pruritus | | | | | | | | | |
| n (%) | | | | | | | | | |
| Injection | 0 | 0 | 2 | (33.3) | 2 (33.3) | 0 | 4 | (12.9) | 0 |

Site
Swelling
n (%)

With rapid absorption and an estimated half-life of up to 127 days, STAR-0215 was found to have a favorable safety profile and to demonstrate a robust inhibition of plasma kallikrein for at least 84 days (at least 3 months) after a single subcutaneous dose in healthy subjects. These results demonstrate STAR-0215's potential as a long-acting preventative therapy for, e.g., HAE.

Additional cohorts of 600 mg IV bolus and 1,200 mg subcutaneous were added to the trial to evaluate the potential for dosing every 6 months. As shown in FIG. 21, the data for the 1,200 mg cohort available at 84 days showed the mean concentration of STAR-0215 was 72.7 μg/mL, well above the 12 μg/mL threshold believed necessary for HAE inhibition. At 28 days, the data for the 600 mg i.v. cohort shows the mean concentration of STAR-0215 was 75.4 μg/mL, well above the 12 μg/mL threshold believed necessary for HAE inhibition. As shown in FIG. 22, the concentration of STAR-0215 at Day 84 for the 300 mg dose suggests the potential for therapeutic dosing at least every three months, whereas the STAR-0215 concentration data for 600 mg SC, and extrapolating from the data for the 1,200 mg SC doses, as well as the 600 mg IV dose contemplates the potential for therapeutic dosing (maintenance of blood concentration above 12 μg/mL) at least, e.g., every six months or more.

Study data showed that STAR-0215 demonstrates dose-dependent concentrations, rapid absorption, slow clearance, and median t_{1/2} of 69-127 days. At Day 84, median concentrations remained 1×, 3×, and 6× above 12 μg/mL (potential efficacy threshold) after 300, 600, and 1,200 mg SC, respectively. Suppression of cHMWK formation consistent with plasma kallikrein inhibition was achieved. According to the data obtained from healthy subjects, it is contemplated that STAR-0215's favorable safety profile, long half-life, and durable PD may provide a potential HAE therapy with robust attack suppression and low treatment burden.

### Example 5: Phase 1b/2 Clinical Study

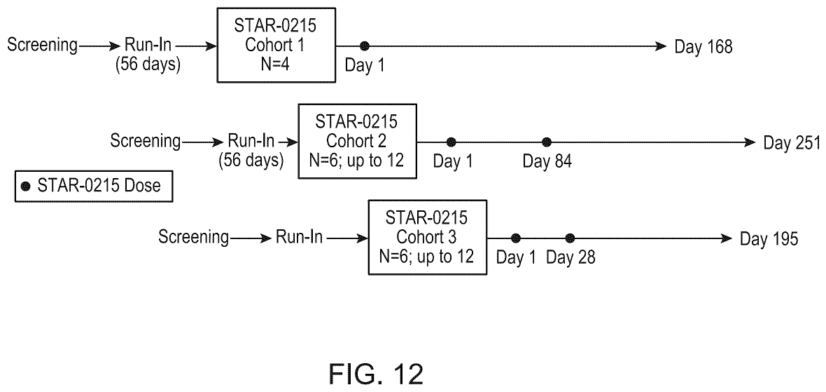

A Phase 1b/2 single and multiple dose study to assess the safety, tolerability, clinical activity, pharmacokinetics, pharmacodynamics, and immunogenicity of STAR-0215 in participants with Hereditary Angioedema (HAE) is planned. A schematic showing an overview of the clinical trial protocol is shown in FIG. 12.

The primary objective of the study is to assess the safety and tolerability of subcutaneous (SC) administration of single and multiple doses of STAR-0215 in participants with Type I or Type II hereditary angioedema (HAE). Primary endpoints of the study will be to assess incidence of adverse events, changes in vital signs, electrocardiogram (ECG) findings, physical examination findings, and clinical laboratory evaluations.

Secondary objectives of the study include assessing the clinical activity of SC administration of single and multiple doses of STAR-0215 in participants with Type I or Type II HAE, characterizing the pharmacokinetics (PK) of SC administration of single and multiple doses of STAR-0215 in participants with Type I or Type II HAE, characterizing the pharmacodynamics (PD) of SC administration of single and multiple doses of STAR-0215 in participants with Type I or Type II HAE, and assessing the immunogenicity of SC administration of single and multiple doses of STAR-0215 in participants with Type I or Type II HAE.

Secondary Endpoints include assessment of (i) change from baseline in monthly HAE attack rate, the incidence of HAE attack severity (mild, moderate, and severe), (ii) the duration of HAE attack (shorter than 12 hours, 12 to 24 hours, 24 to 48 hours, and longer than 48 hours), (iii) the number of HAE attacks requiring on demand therapy, (iv) time to first HAE attack after first and last dosing, (v) concentration of STAR-0215 and the derived PK parameters, (vi) change in plasma kallikrein activity (plasma levels of cleaved HMWK), and (vii) formation of anti-drug (STAR-0215) antibodies.

Exploratory Objectives include evaluating the impact of STAR-0215 on serum or plasma biomarkers related to disease mechanism of action or effect of STAR-0215 and conducting an exploratory assessment of the effect of STAR-0215 on health-related quality of life in participants with Type I or Type II HAE. Exploratory endpoints include assessment of changes from baseline in biomarkers related to disease mechanism of action or therapeutic effect of STAR-0215 and changes from baseline in the Angioedema Quality of Life total and domain scores.

### Methodology and Study Design:

The study will be a Phase 1b/2, multicenter, single and multiple dose study to assess the safety, tolerability, clinical activity, PK, PD, and immunogenicity of STAR-0215 in participants with Type I or Type II HAE.

Properly consented participants will undergo screening assessments according to the inclusion and exclusion criteria and immediately enter the Run-In period. During the Run-In period, HAE attack information will be collected to determine if a participant meets the eligibility requirement of experiencing at least 2 HAE attacks during this time period and to establish a baseline prior to STAR-0215 administration. If a participant has an HAE attack during the Run-In period before Day 1 (first dose of STAR-0215), all signs and symptoms of the attack must be resolved before dosing A washout period for on-demand treatment is not required. The Run-In period may be extended after consultation with the Medical Monitor. In Cohorts 2 and 3, if a participant has an attack immediately before the second dose (Day 84 for Cohort 2; Day 28 for Cohort 3), signs and symptoms of the HAE attack should be improving participant report prior to STAR-0215 administration; a washout period for on-demand treatment is not required before the second dose is administered.

The dose regimens and dose levels for each cohort are outlined in Table 10 below. There will be no placebo group in this study.

TABLE 10

| | Dose 1 | | Dose 2 | | |
|---|---|---|---|---|---|
| | STAR-0215 | | STAR-0215 | | |
| | Dose Level | Dosing | Dose Level | Dosing | # |
| Cohort | (mg) | Day | (mg) | Day | Participants |
| 1 | 450 | 1 | NA | NA | 4 |
| 2 | 600 | 1 | 300 | 84 | 6; Up to 12 |
| 3 | 600 | 1 | 600 | 28 | 6; Up to 12 |

In Cohort 1, each participant who has completed the Run-In period and is eligible for STAR-0215 administration will receive 1 dose of 450 mg of STAR-0215 on Day 1. When Cohort 1 has completed dosing, the Safety Review Committee will review the cohort's cumulative safety data from the Screening/Run-In period through 21 days after dosing. Progression to Cohort 2 dosing will occur if there are no concerning safety signals. In Cohort 2, each eligible participant will receive 2 doses of STAR-0215, the first dose of 600 mg on Day 1 and the second dose of 300 mg on Day 84. When 6 participants in Cohort 2 have received Dose 1, progression to dosing of Cohort 3 will begin. In Cohort 3, each eligible participant will receive 2 doses of STAR-0215, the first dose of 600 mg on Day 1 and the second dose of 600 mg on Day 28. Provided no safety concerns are noted from a cumulative review of safety data from this study and the ongoing first-in-human study described in Example 4, other dose levels and dosing regimens may be explored in additional cohorts. STAR-0215 will be administered by bolus injection subcutaneously (SC) into the abdomen. There will be 4 participants in Cohort 1, 6 participants in Cohort 2, and 6 participants in Cohort 3. Based on the results of interim analyses, up to 6 additional participants may be added to Cohort 2 and/or Cohort 3 (maximum of 12 participants total in Cohorts 2 and 3). Study assessments will be performed through 6 months after the last dose of STAR-0215 in all cohorts (Day 168 in Cohort 1; Day 251 in Cohort 2; Day 195 in Cohort 3). Safety assessments (including adverse events (AEs), clinical laboratory evaluations, physical examinations, electrocardiograms (ECGs), and vital signs), and clinical activity, PK, PD, immunogenicity, biomarker, and quality of life assessments will be conducted.

The starting dose of STAR-0215 for participants with HAE in this study is based on PK/PD modeling. The information evaluated for dose selection for this study included: (1) estimated plasma kallikrein inhibition required for HAE attack suppression based on literature reports and human PK models (empirical and minimal physiologically-based PK (mPBPK)). Based on the totality of the PK/PD modeling, a dose level of 450 mg is considered safe and is estimated to provide adequate target coverage for HAE attack suppression in participants with HAE for a prolonged duration. Therefore, 450 mg is selected to be the starting dose in this Phase 1b/2 study. The planned dosing regimen for Cohort 2 is 600 mg followed by 300 mg 84 days later. Based on PK/PD modeling, this regimen, in which the initial dose is a loading dose, has the potential to rapidly boost Cₘᵢₙ concentrations to levels believed to provide robust and durable inhibition of plasma kallikrein that may translate into immediate and sustained clinical reductions in HAE attacks. The planned dosing regimen for Cohort 3 is 600 mg followed by 600 mg 28 days later. This regimen includes an initial dose on day 1 and a second dose on day 28, expected to result in higher STAR-0215 concentrations during the follow-up period. Upon review of emerging data from the ongoing Phase 1a trial and Cohorts 1, 2, and 3 from this trial, other dose levels and dosing regimens in additional cohorts may be explored.

Single doses of up to 1,200 mg are being evaluated in the ongoing Phase 1a study (Example 4) in healthy adult participants. Initial clinical PK data from the Phase 1a study support the administration of STAR-0215 in humans at a dose level of at least 1,600 mg, 1,600 mg for SC administration and 1,200 mg for IV administration. The study intends to enroll up to 28 participants with HAE. If a participant experiences HAE attacks during the study, they will be permitted standard-of-care on demand treatment as prescribed by their physician.

### Inclusion Criteria

The Inclusion Criteria are that the subject is at least 18 years of age at the time of Screening and:
1. Willing and able to read, understand, and sign the institutional review board (IRB)/independent ethics committee (IEC)/research ethics committee (REC) approved informed consent form (ICF).
2. Documented diagnosis of HAE (Type I or II). All of the following must be met:
3. Documented clinical history consistent with HAE (e.g. SC or mucosal, nonpruritic swelling episodes without accompanying urticaria).
4. C1-INH antigen or functional level less than 40% of the normal level. Participants with antigen or functional C1-INH level 40% to 50% of the normal level may be eligible if they also have a C4 level below the normal range and a family history consistent with HAE Type I or II. Testing for C1-INH and C4 will be performed at Screening. Historical test results for C1-INH may be used to confirm eligibility.
5. Age at reported onset of first angioedema symptoms 30 years of age or younger, or a family history consistent with HAE Type I or II.
6. Agree not to receive a dose of any vaccine within 7 days before or after STAR-0215 administration.
7. Participants of childbearing potential must have a negative serum pregnancy test at Screening, must be not pregnant or breastfeeding at Screening, and agree to use one of the protocol defined forms of highly effective contraception during the study.
Note: Participants who are not capable of becoming pregnant (e.g. surgically sterile or post-menopausal), per the protocol, are not required to use any form of contraception during the study.
8. Participants capable of producing sperm who have partners of childbearing potential must agree to use one of the forms of the protocol defined contraception during the study, unless the participant is azoospermic or their partner is surgically sterile (as defined in the protocol).
9. Agree not to donate or store sperm or engage in any other activity intended to produce sperm for the purpose of insemination during the study, unless such sperm were collected before the administration of the first dose.
10. Agree not to donate or store eggs or undergo assisted reproductive or fertility treatment during the study.
11. Experienced at least 2 HAE attacks during the Run-In period, as confirmed by an investigator based on meeting the protocol-specified definition of an HAE attack.

### Exclusion Criteria

Exclusion Criteria for the study are as follows:
1. Any concomitant diagnosis of another form of chronic angioedema, such as acquired C1 inhibitor deficiency, HAE with normal C1-INH (also known as HAE Type III), idiopathic angioedema, or angioedema associated with urticaria.
2. Any exposure to an investigational drug or device within 90 days or 5 half-lives (whichever is longer) prior to Screening.
3. Exposure to a monoclonal antibody or recombinant protein bearing a Fc domain (such as a soluble receptor-Fc fusion protein) within 5 half-lives prior to Screening.
4. Use of therapies prescribed for the prevention of HAE attacks prior to Screening:
   a. lanadelumab within 90 days
   b. berotralstat within 21 days
   c. all other prophylactic therapies, within 7 days
5. Any exposure to angiotensin-converting enzyme (ACE) inhibitors or any estrogen containing medications with systemic absorption (such as hormonal contraceptives or hormone replacement therapy) within 28 days prior to Screening.
6. Any exposure to androgens (e.g. stanozolol, danazol, oxandrolone, methyltestosterone, testosterone) within 7 days prior to Screening.
7. History of chronic viral infection with positive test for HIV, hepatitis B surface antigen (HBsAg), or hepatitis C virus (HCV) that has not been adequately cured.
8. Active liver disease (e.g. acute or chronic hepatitis B or C, alcoholic or non-alcoholic steatohepatitis).
9. Any of the following liver function test abnormalities: alanine aminotransferase (ALT) above 3 times the upper limit of normal (ULN), or aspartate aminotransferase (AST) above 3 times the ULN, or total bilirubin above 1.5 times the ULN (unless participant has known Gilbert's Syndrome).
10. History of drug or alcohol abuse in the 12 months before Screening.
11. Participant has ongoing cancer, except for the following: basal cell carcinoma of the skin. Ongoing cancer is defined as requiring therapy or intervention to or prevent recurrence/progression of disease.
12. Known sensitivity to the ingredients in STAR-0215.
13. Participant is employed by or is an immediate family member of the Sponsor or study site staff.
14. Participant has any medical or psychiatric condition that, in the opinion of the Investigator, may compromise their safety or compliance, preclude successful conduct of the study, or interfere with interpretation of the results (e.g. a significant pre-existing illness or other major comorbidity that the Investigator considers may confound the interpretation of study results).

STAR-0215 drug product will be supplied as a sterile, preservative-free solution for SC injection in a single-use vial at a concentration of 150 mg/mL. It will be administered in a 300 mg, 450 mg, or 600 mg dose. Individual participants will receive 1 dose of STAR-0215 (Cohort 1; Day 1) or 2 doses of STAR 0215 (Cohort 2; Days 1 and 84 and Cohort 3; Days 1 and 28). The duration of study participation for each participant, including the Screening and Run-In period is up to 224, 307, or 251 days, in Cohorts 1, 2, and 3, respectively. The study is comprised of 3 periods as follows:
a. Run-In: approximately 56 days
b. Treatment Period: Individual participants will receive STAR-0215 on Day 1 (Cohort 1), Day 1 and 84 (Cohort 2), or Day 1 and 28 (Cohort 3).
c. Follow-up Period: approximately 6 months after the last dose of STAR-0215, Day 168 for Cohort 1, Day 251 for Cohort 2, and Day 195 for Cohort 3.

Defining an event as an HAE attack will be based on an assessment of the signs and symptoms of an event against predefined criteria and Investigator clinical judgment. To be considered an HAE attack, the Investigator must confirm that the event has signs or symptoms consistent with an attack that meet at least 1 of the following criteria:
Peripheral angioedema: cutaneous swelling involving an extremity, the face, neck, torso, and/or genitourinary region
Abdominal angioedema: abdominal pain, with or without abdominal distention, nausea, vomiting, or diarrhea
Laryngeal angioedema: stridor, dyspnea, difficulty speaking, difficulty swallowing, throat tightening, or swelling of the tongue, palate, uvula, or larynx

Only meeting at least 1 of the criteria above is not sufficient to confirm an attack. The Investigator will employ his/her clinical judgment to evaluate other features of the event before confirming an attack occurred. Examples of features that may lead to an event not being confirmed as an attack are symptoms that are not consistent with an HAE attack, the event's duration is longer than typical for an HAE attack, or the event has a plausible alternative etiology. Prodromal symptoms or participant-reported use of on-demand HAE attack treatment alone are not sufficient to define an event as an HAE attack.

Each HAE attack must be unique. To be distinct from the previous attack, the new symptoms must occur at least 24 hours after resolution of the prior attack's symptoms. Attack resolution is defined as the subject no longer having symptoms of the attack.

Attack severity will be graded by the Investigator per the following definitions:
Mild: Transient or mild discomfort
Moderate: Mild to moderate limitation in activity, some assistance with daily activities needed
Severe: Marked limitation in activity, assistance with daily activities required

### Criteria for Evaluation

Safety will be assessed by adverse events, vital sign assessments, ECG findings, physical examination findings, and clinical laboratory evaluations, including chemistry, hematology, coagulation, and urinalysis.

Clinical activity will be assessed by weekly collection of investigator-confirmed HAE attacks and accompanying HAE attack information.

Blood samples will be collected for pharmacokinetic evaluation to measure the concentration of STAR 0215 before and after STAR 0215 administration. The concentration of STAR-0215 will be measured using the most current validated bioanalytical method.

Blood samples will be collected for pharmacodynamic analysis to assess plasma kallikrein inhibition associated with STAR-0215 administration. The samples will be evaluated using an appropriate validated assay to measure plasma kallikrein activity.

Blood samples will be collected for immunogenicity analysis to measure anti-drug (STAR-0215) antibodies. The detection and characterization of antibodies to STAR-0215 will be measured using the most current validated bioanalytical method.

Mechanistic and disease biomarkers may be evaluated in serum and plasma. These evaluations may provide a better understanding of the disease mechanism of action or factors related to an HAE attack, as well as biomarkers that may change as a result of STAR-0215 administration or that affect the response to STAR-0215. Assessments may include, but are not limited to, plasma kallikrein and prekallikrein concentrations in circulation, alternative measures of plasma kallikrein activity, functional C1-INH levels over time, and other biomarkers in the contact activation, complement, coagulation, fibrinolysis pathways, or other related disease pathways.

Whole blood samples will be collected in a PAXgenex® blood RNA to isolate RNA for assessing expression levels of genes or pathways associated with HAE, such as the contact activation or complement pathway, or expression transcripts that may change in response to STAR-0215 administration. These assessments may be performed using technologies such as microarray and RNA-seq, or other sequencing technologies.

With participant consent, whole blood will be collected in a PAXgene® blood DNA tube for pharmacogenomic assessments including genetic polymorphisms (baseline) and epigenetic modifications associated with HAE, or that impact a participant's response to STAR-0215 or result from STAR-0215 administration.

HAE cases (Type I and Type II) are caused by mutations in the SERPING1 gene that lead to a reduction in the amount or function of C1-INH encoded by this gene. Variants in the SERPING1 gene or other genes in the contact activation, kinin, coagulation or related pathways have been shown to influence the severity of HAE. The assessment of epigenetics (such as DNA methylation) and genetic variation in genes related to HAE etiology or drug disposition may be beneficial in understanding participant response to STAR-0215 administration. Whole genome sequencing will not be performed.

The Angioedema Quality of Life Questionnaire (Weller et al., 2012, Allergy, 67 (10); 1289-1298) is a validated, angioedema-specific, patient-reported outcome measure that is self-administered and used to evaluate the impact of recurrent angioedema on patients' quality of life. It is a 17-item questionnaire covering 4 domains (functioning, fatigue/mood, fears/shame, and nutrition). Each item has 5 answer options (scored 1 to 5), with higher scores indicating a more adverse effect. Raw scores are transformed into a linear scale that ranges from 0 to 100, with a score of 100 indicating the worst possible impairment in quality of life. The minimally clinically important difference is defined as a change of 6 points from baseline.

Data will be summarized using descriptive statistics (mean, median, standard deviation, minimum and maximum) or frequency counts and percentages, as appropriate. Results will be presented by cohort.

Unless otherwise specified, all analyses of HAE attack data will be presented similarly during the Run-In and treatment periods. Data will be analyzed for all investigator confirmed HAE attacks.

The frequency and duration of HAE attacks will be presented with descriptive statistics overall and by participant. The following categories will be presented for attack duration (shorter than 12 hours, 12 to 24 hours, 24 to 48 hours, and longer than 48 hours).

Attack severity categorized as Mild, Moderate or Severe will be presented overall and by participant. The number and type of on-demand therapies used to treat HAE attacks will be summarized. The proportion of HAE attack-free subjects during the treatment period will be summarized. The time normalized number of investigator-confirmed HAE attacks per month (attack rate) will be presented at 4-week intervals and at the end of the study. The mean monthly rate and 95% confidence interval will be presented.

To assess HAE attacks within a participant, the actual and percentage change from baseline (Run-In period) in monthly attack rate with respect to on-treatment attack rate will be calculated. The mean change and 95% confidence interval will be presented. The proportion of participants with a clinical response to treatment with greater than or equal to 40%, 50%, 70%, 90%, and 100% reduction of HAE attacks relative to baseline will be presented. Time to first HAE attack after first and last dose of STAR-0215 will be presented using Kaplan-Meier estimates.

Quality of life will be assessed by means of the Angioedema Quality of Life Questionnaire (AE-QoL). Descriptive statistics for changes from baseline to each visit in the total score will be presented. A 95% confidence interval for the change will also be presented. Individual domain scores will be presented similarly.

In preparation for future clinical development of STAR-0215 and subsequent study planning, 2 interim analyses of data in accumulating cohorts are planned. The first interim analysis will occur after the fourth participant in Cohort 1 reaches the Day 84 visit and will include all available data from Cohorts 1 and 2. The second interim analysis will occur after the 4th participant in Cohort 3 reaches 84 days after the second dose of STAR-0215 (Day 111) and will include all available data from Cohorts 1, 2, and 3. Details of any addition interim analysis that may be conducted as warranted will be specified in the in the Interim Analysis Statistical Analysis Plan (SAP). The sponsor will review the safety, PK, PD, immunogenicity, and efficacy results of each interim analysis and may decide to enroll up to 6 additional participants in Cohort 2 and/or Cohort 3 (up to 12 participants in total in each). If no safety concerns are noted following interim analysis, other dose levels (300 mg to 1,200 mg) and dosing regimens (single or multiple doses) may be explored in additional cohorts. All analyses will be predefined in the SAP as applicable.

Participants may have the opportunity to receive STAR-0215 as a participant in a long-term open label study under a separate protocol.

### Results

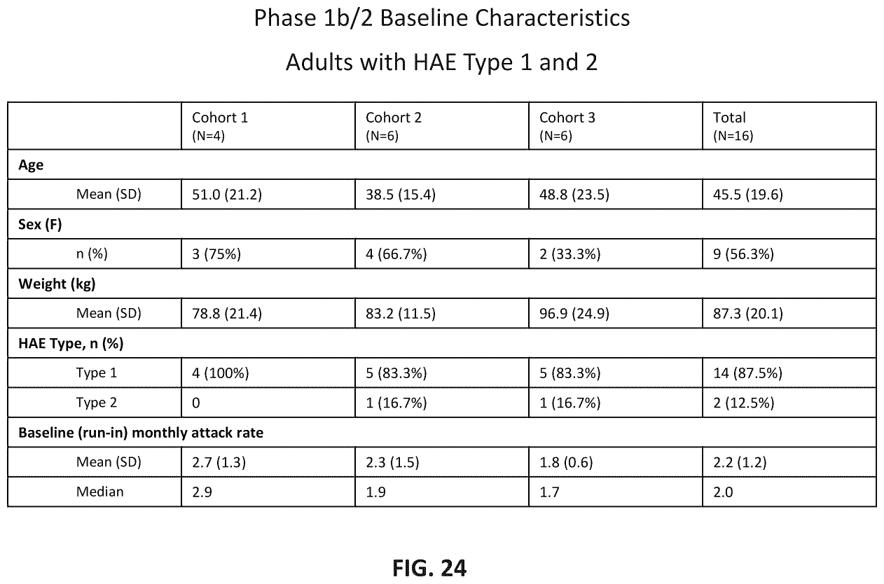

Initial results demonstrated that STAR-0215 has a favorable safety and tolerability profile, achieved a mean monthly attack rate reduction of 90-96% for up to 6 months of follow up, and support both every three- and six-month dosing regimens. The baseline characteristics of the enrolled subjects in each of Cohorts 1, 2, and 3 can be found in FIG. 24. As of the cutoff for the initial data presented herein, all 4 participants in Cohort 1 had completed 3 and 6 months of follow-up. In cohort 2, all 6 participants had completed 3 months of follow-up and 3 of the 6 had completed 6 months of follow-up. In cohort 3, 4 of 6 participants had completed 3 months of follow-up and no participants had completed 6 months of follow-up.

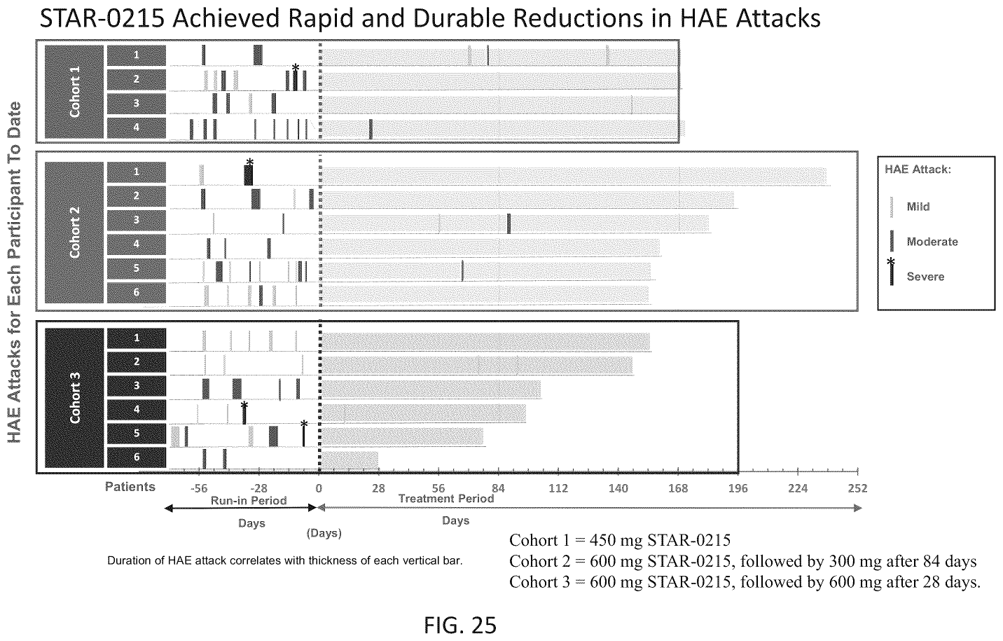

The results showed that STAR-0215 achieved rapid and durable reductions in HAE attacks in subjects in all cohorts. FIG. 25 is a swimmer's lane plot of the number, duration, and severity of HAE attacks for every subject in each of Cohorts 1, 2, and 3. Data is shown for both the 8 week run-in period and the treatment period up to the cut off. Treatment data is shown through 6 months (168 days) for all subjects in Cohort 1. For Cohort 2, only 3 of 6 subjects had reached 6 months since the commencement of treatment by the cut-off. For Cohort 3, only 4 of 6 subjects had reached 3 months (84 days) since treatment by the cut-off. At the time of cut-off, no subjects in Cohort 3 had reached 168 days since the commencement of treatment. A marked decrease in the number, duration and severity of HAE attacks was observed in all subjects in all cohorts over the treatment period, with, surprisingly, several subjects remaining attack free from the commencement of treatment.

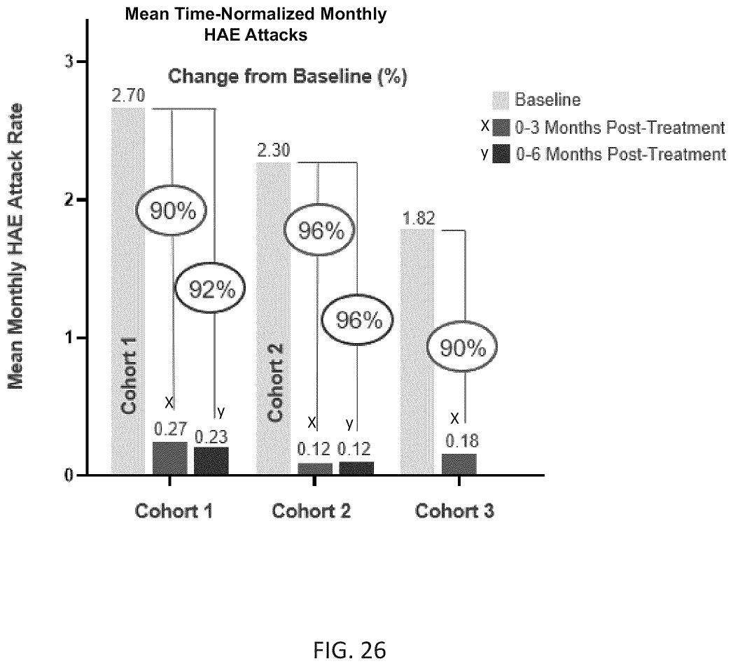

Most surprisingly, there was a 90-96% reduction in the monthly attack rate after administration of STAR-0215 compared to baseline from 0-3 months and from 0-6 months for all cohorts. For example, as shown in FIG. 26, in Cohort 1 there was a 90% reduction in monthly attack rate compared to baseline for the 0-3 month post-treatment time frame, and a 92% reduction in monthly attack rate compared to baseline for the 0-6 month post-treatment time frame. In Cohort 2 there was a 96% reduction in monthly attack rate compared to baseline for the 0-3 month post-treatment time frame, and a 96% reduction in monthly attack rate compared to baseline for the 0-6 month post-treatment time frame. For Cohort 3 there was a 90% reduction in monthly attack rate compared to baseline for the 0-3 month post-treatment time frame. As no subjects had yet reached 6 months from commencement of treatment, data was not available for the 6 month change from baseline. Based on all available data as of the cut-off, the cumulative all-available follow-up attack rate reduction was 91-94%. Based on these data, the monthly HAE attack rate reduction of 90-96% through three months and 92-96% for through six months exceeded initial expectations of a 75-90% reduction in monthly attack rate in each of the cohorts. The results also demonstrate that rapid and robust attack rate reduction is possible for at least three months, and in some cases up to 6 months after a single dose of STAR-0215.

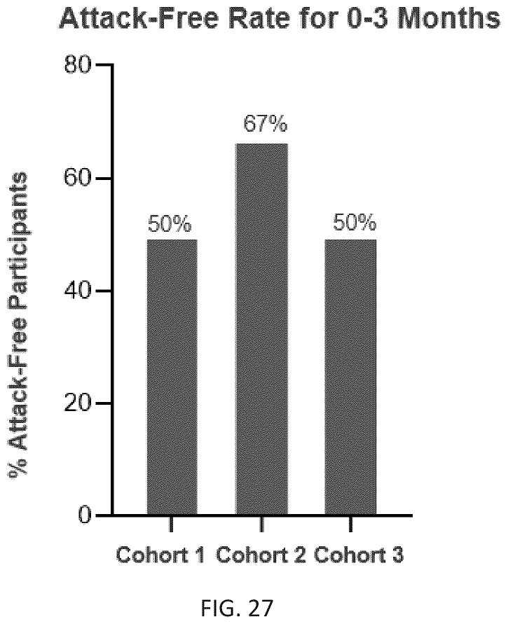

Another significant finding was that a substantial percentage of participants were HAE attack-free while receiving STAR-0215 treatment. For the first 3 months following STAR-0215 treatment, 50-67% of participants were attack free, and in Cohort 2, of those who made it to the 6 month follow-up before the cut off, 67% were attack free. For example, as shown in FIG. 27, at 3 months 50% of subjects in Cohort 1 (N=4) were attack free, 67% of subjects in Cohort 2 (N=6) were attack free, and 50% of the subjects who had progressed through day 84 (3 months; N=4) were attack free. Further, in Cohort 2, 67% of subjects who had progressed through 6 months (day 168) were attack free (N=3) (See FIG. 25). Cohort 1 had a 25% attack-free rate through 6 months after a single dose of 450 mg. Accordingly, administration of STAR-0215 was demonstrated to be effective in eliminating the burden of HAE attacks in subjects suffering from HAE for at least three months and even for at least six months based on the dosing regimens tested.

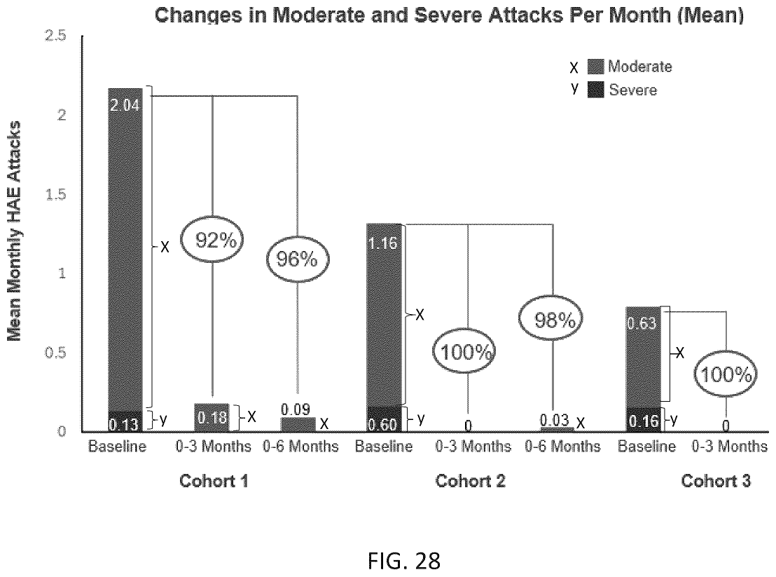

Further, initial data from the study demonstrated that STAR-0215 reduced the severity of HAE attacks experienced by subjects in each of the three cohorts through three and six months. The data are shown in FIG. 28. In fact, there was a 92-100% decrease in moderate or severe attacks across all three cohorts at 3 months and 96-98% decrease in Cohorts 1 and 2 by 6 months. In Cohort 1 (N=4), there was a 92% reduction in moderate HAE attacks per month over 3 months and a 96% reduction in moderate HAE attacks per month over 6 months, with a 100% reduction in severe HAE attacks per month over 3 and 6 months. In Cohort 2 (N=6), 6 participants had 3 month and 3 participants had 6 month follow-ups, and there was a 100% reduction in moderate and severe HAE attacks per month over 3 months (N=6) and a 98% reduction in moderate HAE attacks per month and a 100% reduction in severe HAE attacks over 6 months (N=3). In Cohort 3, there was a 100% reduction in both the severe and moderate monthly attack rates at 3 months based on the 4 participants who progressed to the 3 month (84 days) follow-up before the cut off. No participants in Cohort 3 had progressed to 6 month follow-up as of the data cutoff. It was remarkable that no severe attacks were seen in any cohort after administration of STAR-0215 as of the time of the data cutoff. Accordingly, the results demonstrate the effectiveness of STAR-0215 to reduce the severity of HAE attacks, and to eliminate severe attacks.

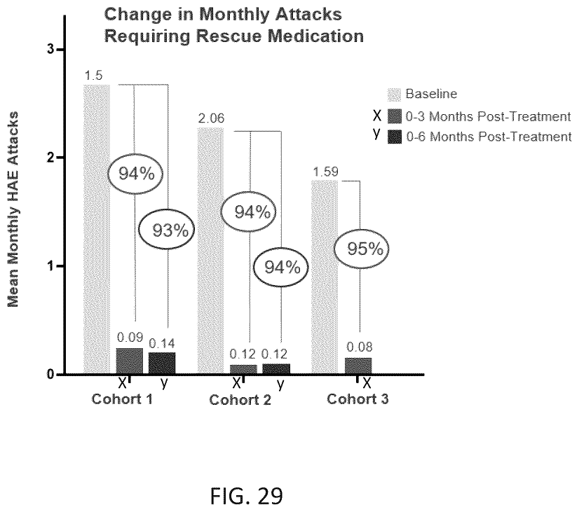

The initial data also showed that STAR-0215 reduced the number of HAE attacks requiring the subject to take a rescue medication (i.e., an on-demand therapy to treat the attack). In fact, the results indicated that 93-95% fewer attacks required rescue medication after commencing STAR-0215 treatment compared to baseline at 3 and 6 months post-treatment. The results are shown in FIG. 29. Specifically, in Cohort 1, the monthly rate of attacks requiring rescue medication was reduced by 94% from baseline 0-3 months post-treatment and by 93% 0-6 months post-treatment compared to baseline. In Cohort 2, the monthly rate of attacks requiring rescue medication was reduced by 94% from baseline 0-3 months post-treatment (N=6) and by 94% 0-6 months post-treatment compared to baseline (N=3). In Cohort 3, the monthly rate of attacks requiring rescue medication was reduced by 95% from baseline 0-3 months post-treatment. This reduction is important to show the positive impact of STAR-0215 on subjects' quality of life and also by reducing the costs associated with taking an on-demand therapy.

STAR-0215 was found to be well-tolerated and demonstrated a favorable safety profile. No reports of injection site pain or clinically significant change in laboratory parameters or electrocardiograms (ECGs) were recorded. A summary of treatment-emergent adverse events (TEAEs) is found in Table 11 below. TEAEs are shown that occurred in at least two participants, whereas related TEAEs are all shown. The two related TEAEs were both mild; one was a case of mild dizziness on day 6 after the first dose in Cohort 2 and the other was a transient injection site reaction (rash) 5 days after the second dose in Cohort 3, lasting less than 1 day.

TABLE 11

| | Cohort 1 | Cohort 2 | Cohort 3 | Total |
|---|---|---|---|---|
| | (N = 4) | (N = 6) | (N = 6) | (N = 16) |
| Treatment-Emergent | 6 | 1 | 1 | 8 |
| Adverse Events (TEAE)* | | | | |
| Contusion | 3 | — | — | 3 |
| Nasopharyngitis | 1 | 1 | 1 | 3 |
| Headache | 2 | — | — | 2 |
| Related TEAEs | — | 1 | 1 | 2 |
| Dizziness | — | 1 | — | 1 |
| Injection Site Rash | — | — | 1 | 1 |
| N with Serious Adverse | — | — | — | — |
| Events | | | | |
| N who have discontinued | — | — | — | — |
| due to TEAE | | | | |

Related TEAE = TEAE deemed at least possibly related to study drug by principal investigator.

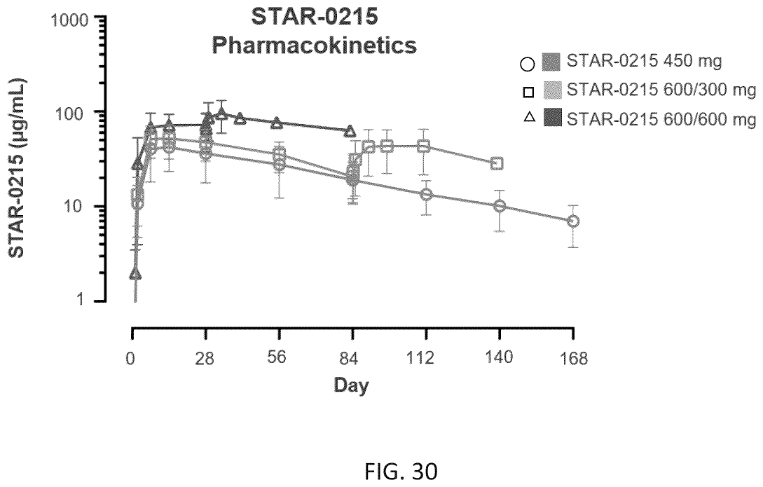

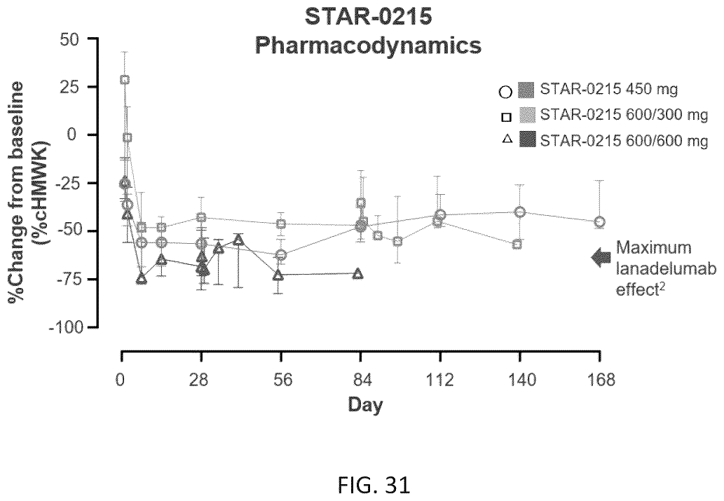

The pharmacokinetic and pharmacodynamic data for STAR-0215 were consistent with a clinical benefit for administration of STAR-0215. The pharmacokinetic data for STAR-0215 serum concentration is shown in FIG. 30. The concentrations remained above 12 μg/mL for more than 3 months in Cohort 1 and well above this target threshold in Cohorts 2 and 3 for the duration of available follow-up. The initial pharmacodynamic data is presented in FIG. 31 which showed sustained inhibition of plasma kallikrein activity as measured by the rate of inhibition of cleavage of high molecular weight kininogen (cHMWK) as compared to baseline. The results show that there was sustained inhibition of plasma kallikrein activity similar to lanadelumab and suggest that treatment with STAR-0215 has features that make it a superior choice to treatment with lanadelumab. A summary of comparators between the initial results from this Phase 1b/2 trial and the Phase 3 trial of lanadelumab are provided in Table 12 below.

TABLE 12

| | | | | | |
|---|---|---|---|---|---|
| | | Reduction | | | |
| Monthly | Attack- | in | Reduction | | |
| Attack | Free Rate | Moderate | in Attacks | | |
| Rate | for at | and Severe | Requiring | Injection | Doses |
| Reduction | Least 1^{st} | Attacks | Rescue | Site | Per |
| (mean) | 3 Months | per Month | Medication | Pain | Year |

Phase 1b/2 Initial Results Summary

| | | | | | | |
|---|---|---|---|---|---|---|
| STAR-0215, | 90-96% | 50-67% | 92-100% | 93-95% | 0% | 2 or 4 |
| Summary | | | | | | |
| Cohort 2: 600 mg | 96% | 67% | 98% | 94% | 0% | 4 |
| Day 1, 300 mg Day | | | | | | |
| 84, through 6 M | | | | | | |

Lanadelumab Phase 3 Results Summary

| | | | | | | |
|---|---|---|---|---|---|---|
| Lanadelumab 300 | 87% | 44% | 87% | 87% | 52% | 26 |
| mg Q2W | | | | | | |
| Lanadelumab 300 | 73% | 31% | 73% | 74% | 31% | 13 |
| mg Q4W | | | | | | |

The results from lanadelumab are from a separate, Phase 3, placebo-controlled trial in adults and adolescents with Type 1 or 2 HAE (n=125). Lanadelumab efficacy endpoints were compared with the placebo group using a Poisson regression model after 6-months treatment period. The comparison presented between STAR-0215 and the lanadelumab data represents a cross-trial comparison and does not involve data from a head-to-head clinical trial. STAR-0215 efficacy endpoints are mean change from baseline.

Accordingly, the data suggest STAR-0215 has potentially increased clinical efficacy over landelumab with a lower treatment burden due to once every 3 or 6 month dosing and no injection site pain.

The observed efficacy, PK, PD, and safety and tolerability profile of STAR-0215 establish proof of concept for using STAR-0215 to treat HAE and support advancement of STAR-0215 into Phase 3 development, including for evaluation of every three months (4 doses per year) and every six months (2 doses per year) dosing protocols. Plans are underway to evaluate a 600 mg dose followed by a 300 mg dose after 84 days in Phase 3, and to continue the 300 mg dose every 3 months thereafter in an open-label extension study if clinical benefit is shown. The doses will be administered subcutaneously. This dosing regimen is supported by the data which showed for Cohort 2 a 96% reduction in monthly attack rate, 98-100% reduction in moderate/severe attacks, a 94% reduction in use of rescue medications, 100% of patients being attack-free in first month and 67% of patients being attack-free through 6 months.

### Example 6: Open Label Extension of Phase 2 Clinical Study

The primary objective of this study is to assess long-term safety and tolerability of STAR-0215 in participants with Type 1 or Type II HAE. Primary endpoints include incidence of adverse events, changes in vital signs, ECG findings, physical examination findings, and clinical laboratory evaluations.

Secondary objectives include assessing the long-term efficacy of STAR-0215 in participants with Type I or Type II HAE, characterizing the PK, PD, and immunogenicity of STAR-0215 long term dosing in participants with Type I or Type II HAE, and to evaluate the impact of long-term dosing on serum or plasma biomarkers related to disease mechanism of action and to conduct an exploratory assessment of the effect of long-term STAR-0215 dosing on health-related quality of life in participants. Secondary endpoints include change from baseline in monthly HAE attack rate, incidence of monthly HAE attack severity, duration of monthly HAE attacks, the number of HAE attacks requiring on-demand therapy, time to first HAE attack after each dose, the number of attack-free days, proportion of HAE attack-free participants, concentration of STAR-0215 and the derived PK parameters, changes in plasma kallikrein activity, formation of anti-drug antibodies, changes from baseline in biomarkers related to disease mechanism of action or therapeutic effect of STAR-0215, and changes from baseline in the Angioedema-Quality of Life (AE-QoL) total and domain scores.

Patients who completed all visits through 6 months in the Phase 2 study described in Example 5, as well as patients who entered the Phase 2 study Run-in period but did not qualify for the treatment period because they did not meet the minimum number of HAE attacked during the Run-in period are eligible to enroll in this open-label extension study. Up to 56 participants will be enrolled.

Enrolled subjects will receive STAR-0215 on Day 1 in 1 of 2 dosing regimens. Assignment to a dosing regimen is based on a participant's cohort assignment in the Phase 2 trial. After the initial dose (Day 1), participants will either receive STAR-0215 every 3 months (Dosing Regimen 1) or, after an additional dose 1 month later, every 6 months (Dosing Regimen 2). If a participant has an HAE attack in the days immediately before dosing, signs and symptoms of the HAE attack should be improving, per participant report, before STAR-0215 administration. If under Regimen 2 a participant's HAE attacks are not well controlled, the participant may be switched to Regimen 1 after at least 12 months of participation in open-label extension study.

Assessments will be performed at regular intervals throughout the trial, including at each STAR-0215 administration. Assessments will include safety (including AEs, vital signs, ECGs, physical examinations, and clinical laboratory evaluations), efficacy (HAE attack information), PK, PD, immunogenicity, biomarker, and quality-of-life assessments. The frequency of select assessments will taper over the course of the participant's trial participation.

Participants will continue with study drug administration for approximately 5 years or until marketing authorization of STAR-0215, whichever comes first. Following the end of treatment, monitoring will be performed for an additional 16 months after the last dose of STAR-0215, regardless of dosing regimen. This follow-up period consists of 6 remote contacts and two onsite visits. The purpose of this trial is to evaluate the long-term safety and tolerability of STAR-0215 with the dosing regimens anticipated to be effective in prolonged HAE attack suppression.

The dosing regimens that will be tested in this trial are the following:

| Dose | Dose 1 on Day 1/ | | |
|---|---|---|---|
| Level | Month 1 | Continued Dosing | |
| (mg) | Dose Level (mg) | Frequency | |
| 1 | 600 | 300 (starting at Month 4) | every 3 months |
| 2 | 600 | 600 (starting at Month 2) | every 6 months |

These dose regimens are selected based on the potential to maintain STAR-0215 concentrations above 12 μg/mL or 80 nM, which is the plasma concentration expected to prevent HAE attacks in patients with HAE. Simulations of STAR-0215 concentrations over time in each of the 3 cohorts in the Phase 1b trial (Cohort 1:450 mg; Cohort 2:600 mg Day 1, 300 mg Day 84; Cohort 3:600 mg Day 1, 600 mg Day 28) from a PK model built with initial Phase 1a healthy participant data are shown in FIGS. 17A-C. As shown in FIG. 17C, the PK model suggests that initial delivery of STAR-0215 intended to achieve higher concentrations than those attainable in Cohort 2 may result in sustained STAR-0215 concentrations above 12 μg/mL for at least 6 months.

Both Regimen 1 and Regimen 2 in this trial utilize an initial dose(s) that has the potential to rapidly boost STAR-0215 concentrations to levels believed to provide sufficient inhibition of plasma kallikrein that may translate into immediate reductions in HAE attacks. Such initial dosing is still considered necessary for the participants who completed the Phase 1b/2 trial because their blood concentration of STAR-0215 is anticipated to be below the therapeutic threshold after the 6-month follow-up period leaving them more susceptible to HAE attacks. In Regimen 1 the initial dose is 600 mg SC on Day 1. Regimen 2 employs two administrations of 600 mg SC, one on Day 1 and one a month later. Following the initial dose(s), the continued dosing at the dose level of 300 mg SC (or 600 mg SC) is anticipated to provide adequate exposure for sustained plasma kallikrein inhibition and HAE attack suppression for the duration of at least 3 months (or 6 months). The rationale for the interval of 1 month between the first and second 600-mg dose in Regimen 2 is to induce STAR-0215 concentrations over a 1-month period, rather than load with 1 higher dose administration on Day 1. Because the current formulation concentration is 150 mg/mL, the design in which two 600 mg doses are administered over 1 month minimizes the injection volume at each dosing day. For example, one 1,200-mg dose on Day 1 would require four 2-mL SC injections. As designed, Regimen 2 requires two 2-mL SC injections on Day 1 and two 2-mL SC injections 1 month later.

Upon review of emerging data from the ongoing Phase 1a and Phase 1b/2 trials and from this trial, other dose levels and dosing regimens may be explored.

### Contemplated Results

It is contemplated that subjects enrolled in this open-label extension study will exhibit a clinical response to treatment with STAR-0215, such as experiencing a statistically significant reduction in the frequency and the number of HAE attacks compared to baseline, and/or remaining free of attacks for a prolonged period of time as a result of receiving STAR-0215 every 3 months or 6 months according to either of the dosing regimens. It is also contemplated that STAR-0215 will be found safe and well-tolerated, that quality of life (QoL) will improve, for example as measured by the AE-QoL score. It is also contemplated that the reduction in frequency of HAE attacks in study subjects from baseline of an expected 1-4 attacks per month (average of 2 attacks per month) will be about 75-90% or more.

### INCORPORATION BY REFERENCE

The entire disclosure of each of the patent and scientific documents referred to herein is incorporated by reference for all purposes.

### Equivalents

The present embodiments are not to be limited in scope by the specific embodiments described herein. Indeed, various modifications in addition to those described herein will become apparent to those skilled in the art from the foregoing description and the accompanying figures.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

### NUMBERED EMBODIMENTS

Embodiment 1. A method of treating a human subject having a plasma kallikrein associated disorder, comprising:
administering a loading dose of 300-2,000 mg of an anti-plasma kallikrein antibody to the subject; and
administering a maintenance dose of 150-1,500 mg of the anti-plasma kallikrein antibody to the subject beginning at least about 2 months after administering the loading dose and then at least about every 2 months thereafter;
wherein the anti-plasma kallikrein antibody comprises:
   i) Complementarity Determining Sequences (CDRs) of a heavy chain variable region having the amino acid sequence of SEQ ID NO: 7, and CDRs of a light chain variable region having the amino acid sequence of SEQ ID NO: 8, wherein the antibody further comprises a constant region comprising an M252Y, an S254T, and a T256E mutation as numbered according to the EU numbering index;
   ii) an immunoglobulin heavy chain variable region comprising the amino acid sequence of SEQ ID NO: 7, and an immunoglobulin light chain variable region comprising the amino acid sequence of SEQ ID NO: 8, wherein the antibody further comprises a constant region comprising an M252Y, an S254T, and a T256E mutation as numbered according to the EU numbering index; or
   iii) an immunoglobulin heavy chain comprising the amino acid sequence of SEQ ID NO: 9 and a light chain comprising the amino acid sequence of SEQ ID NO:10.

Embodiment 2. A method of achieving a circulating blood concentration of an anti-plasma kallikrein antibody in excess of 10 μg/mL for at least two months in a subject having a plasma kallikrein associated disorder comprising administering to the subject a loading dose followed by a maintenance dose of an anti-plasma kallikrein antibody comprising:
i) Complementarity Determining Sequences (CDRs) of a heavy chain variable region having the amino acid sequence of SEQ ID NO: 7, and CDRs of a light chain variable region having the amino acid sequence of SEQ ID NO: 8, wherein the antibody further comprises a constant region comprising an M252Y, an S254T, and a T256E mutation as numbered according to the EU numbering index;
ii) an immunoglobulin heavy chain variable region comprising the amino acid sequence of SEQ ID NO: 7, and an immunoglobulin light chain variable region comprising the amino acid sequence of SEQ ID NO: 8, wherein the antibody further comprises a constant region comprising an M252Y, an S254T, and a T256E mutation as numbered according to the EU numbering index; or
iii) an immunoglobulin heavy chain comprising the amino acid sequence of SEQ ID NO: 9 and a light chain comprising the amino acid sequence of SEQ ID NO:10.

Embodiment 3. A method of treating a human subject having a plasma kallikrein associated disorder, comprising:
administering a loading dose of 300-2,000 mg of an anti-plasma kallikrein antibody to the subject;
administering a first maintenance dose of 150-1,500 mg of the anti-plasma kallikrein antibody to the subject about 1 week to 8 weeks after administering the loading dose;
administering a second maintenance doses of 150-1,500 mg 2-9 months after the first maintenance dose, and
administering subsequent maintenance doses every 2-9 months after the second maintenance dose;
wherein the first maintenance dose, second maintenance dose, and any subsequent maintenance dose are the same dose; and
wherein the anti-plasma kallikrein antibody comprises:
   i) Complementarity Determining Sequences (CDRs) of a heavy chain variable region having the amino acid sequence of SEQ ID NO: 7, and CDRs of a light chain variable region having the amino acid sequence of SEQ ID NO: 8, wherein the antibody further comprises a constant region comprising an M252Y, an S254T, and a T256E mutation as numbered according to the EU numbering index;
   ii) an immunoglobulin heavy chain variable region comprising the amino acid sequence of SEQ ID NO: 7, and an immunoglobulin light chain variable region comprising the amino acid sequence of SEQ ID NO: 8, wherein the antibody further comprises a constant region comprising an M252Y, an S254T, and a T256E mutation as numbered according to the EU numbering index; or
   iii) an immunoglobulin heavy chain comprising the amino acid sequence of SEQ ID NO: 9 and a light chain comprising the amino acid sequence of SEQ ID NO:10.

Embodiment 4. The method of embodiment 2, wherein the loading dose is in a range of 300-2000 mg and the maintenance dose is in a range of 150-1500 mg.

Embodiment 5. The method of any one of embodiments 1-4, wherein the loading dose is in a range of 300-900 mg.

Embodiment 6. The method of any one of embodiments 1-5, wherein the loading dose is in a range selected from 300-350 mg, 350-400 mg, 400-450 mg, or 450-500 mg.

Embodiment 7. The method of any one of embodiments 1-6, wherein the loading dose is in a range selected from 600-900 mg.

Embodiment 8. The method of any one of embodiments 1-7, wherein the loading dose is in a range selected from 300-350 mg, 350-400 mg, 400-450 mg, 450-500 mg, 500-550 mg, 550-600 mg, 600-650 mg, 650-700 mg, 700-750 mg, 750-800 mg, or 850-900 mg.

Embodiment 9. The method of any one of embodiments 1-8, wherein the loading dose is about 400-450 mg.

Embodiment 10. The method of any one of embodiments 1-6, 8, or 9, wherein the loading dose is 300 mg, 325 mg, 350 mg, 375 mg, 400 mg, 425 mg, 450 mg, 475 mg, or 500 mg.

Embodiment 11. The method of any one of embodiments 1-5 or 7-10, wherein the loading dose is selected from 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, or 900 mg.

Embodiment 12. The method of any one of embodiments 1-4, wherein the loading dose is in a range selected from 900-1,200 mg.

Embodiment 13. The method of any one of embodiments 1-4, wherein the loading dose is in a range from 500-2,000 mg.

Embodiment 14. The method of any one of embodiments 1-8, 12, or 13, wherein the loading dose is in a range selected from 350-400 mg, 400-450 mg, 450-500 mg, 500-550 mg, 550-600 mg, 600-650 mg, 650-700 mg, 700-750 mg, 750-800 mg, 850-900 mg, 900-50 mg, 950-1,000 mg, 1,000-1,050 mg, 1,050-1,100 mg, 1,100-1,150 mg, 1,150-1,200 mg, 1,200-1,250 mg, 1,250-1,300 mg, 1,300-1,350 mg, 1,350-1,400 mg, 1,400-1,450 mg, 1,450-1,500 mg, 1,500-1,550 mg, 1,550-1,600 mg, 1,600-1,650 mg, 1,650-1,700 mg, 1,700-1,750 mg, 1,750-1,800 mg, 1,800-1,850 mg, 1,850-1,900 mg, 1,900-1,950 mg, or 1,950-2,000 mg

Embodiment 15. The method of any one of embodiments 1-8, or 12-14, wherein the loading dose is selected from 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1,000 mg, 1,025 mg, 1,050 mg, 1,075 mg, 1,100 mg, 1,125 mg, 1,150 mg, 1,175 mg, 1,200 mg, 1,225 mg, 1,250 mg, 1,275 mg, 1,300 mg, 1,325 mg, 1,350 mg, 1,375 mg, 1,400 mg, 1,425 mg, 1,450 mg, 1,475 mg, 1,500 mg, 1,525 mg, 1,550 mg, 1,575 mg, 1,600 mg, 1,600 mg, 1,625 mg, 1,650 mg, 1,675 mg, 1,700 mg, 1,725 mg, 1,750 mg, 1,775 mg, 1,800 mg, 1,825 mg, 1,850 mg, 1,875 mg, 1,900 mg, 1,925 mg, 1,950 mg, 1,975 mg, or 2,000 mg.

Embodiment 16. The method of any one of embodiments 1-5, 7, 8, or 11-15, wherein the loading dose is 600 mg.

Embodiment 17. The method of any one of embodiments 1-5, 7, 8, or 11-15, wherein the loading dose is 900 mg.

Embodiment 18. The method of any one of embodiments 1-4, or 11-15, wherein the loading dose is 1,200 mg.

Embodiment 19. The method of any one of embodiments 1-18, wherein the maintenance dose is in the range of 250-1,500 mg.

Embodiment 20. The method of any one of embodiments 1-18, wherein the maintenance dose is in the range of 200 mg-1,500 mg.

Embodiment 21. The method of any one of embodiments 1-20, wherein the maintenance dose is 150-450 mg.

Embodiment 22. The method of any one of embodiments 1-21, wherein the maintenance dose is in a range selected from 250-300 mg, 300-350 mg, 350-400 mg, 450-500 mg, 500-550 mg, 550-600 mg, 600-650 mg, 650-700 mg, 700-750 mg, 750-800 mg, 850-900 mg, 900-50 mg, 950-1,000 mg, 1,000-1,050 mg, 1,050-1,100 mg, 1,100-1,150 mg, 1, 150-1,200 mg, 1,200-1,250 mg, 1,250-1,300 mg, 1,300-1,350 mg, 1,350-1,400 mg, 1,400-1,450 mg, or 1,450-1,500 mg.

Embodiment 23. The method of any one of embodiments 1-22, wherein the maintenance dose is in the range of 150-250 mg, or is 150 mg, 175 mg, 200 mg, 225 mg, or 250 mg.

Embodiment 24. The method of any one of embodiments 1-22, wherein the maintenance dose is in a range of 300-450 mg.

Embodiment 25. The method of any one of embodiments 1-22, wherein the maintenance dose is in a range of 300-900 mg.

Embodiment 26. The method of any one of embodiments 1-22, wherein the maintenance dose is in a range of 200-250 mg.

Embodiment 27. The method of any one of embodiments 1-22, 24 or 25, wherein the maintenance dose is 300 mg or 450 mg.

Embodiment 28. The method of any one of embodiments 1-27, wherein the maintenance dose is selected from 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1,000 mg, 1,025 mg, 1,050 mg, 1,075 mg, 1,100 mg, 1,125 mg, 1,150 mg, 1,175 mg, 1,200 mg, 1,225 mg, 1,250 mg, 1,275 mg, 1,300 mg, 1,325 mg, 1,350 mg, 1,375 mg, 1,400 mg, 1,425 mg, 1,450 mg, 1,475 mg, or 1,500 mg.

Embodiment 29. The method of any one of embodiments 1-28, wherein the maintenance dose is one-half the amount of loading dose.

Embodiment 30. The method of any one of embodiments 2-29, wherein the first maintenance dose is administered to the subject about 1-2 weeks after administration of the loading dose.

Embodiment 31. The method of any one of embodiments 2-30, wherein the second maintenance dose is administered about 3-6 months after the first maintenance dose and the subsequent maintenance doses are administered every 3-6 months after the second maintenance dose, wherein the time between the first maintenance dose and second maintenance dose and between the second maintenance dose and subsequent maintenance dose are the same.

Embodiment 32. The method of any one of embodiments 2-31, wherein the second maintenance dose is administered 3 months after the first maintenance dose and the subsequent maintenance doses are administered every 3 months after the second maintenance dose.

Embodiment 33. The method of any one of embodiments 2-31, wherein the second maintenance dose is administered 6 months after the first maintenance dose and the subsequent maintenance doses are administered every 6 months after the second maintenance dose.

Embodiment 34. The method of any one of embodiments 1-33, wherein the loading dose and the maintenance doses are administered subcutaneously to the subject.

Embodiment 35. The method of any one of embodiments 1-33, wherein the loading dose and the maintenance doses are administered intravenously to the subject.

Embodiment 36. The method of any one of embodiments 1-33, wherein the loading dose is administered intravenously and the maintenance doses are administered subcutaneously.

Embodiment 37. The method of embodiments 34 or 36, wherein the subcutaneous dose is administered from a prefilled syringe, autoinjector device, or via an on-body or wearable injection device.

Embodiment 38. The method of embodiments 35 or 36, wherein the intravenous dose is administered as a bolus injection or by infusion.

Embodiment 39. The method of any one of embodiments 1-38, wherein the loading dose maintains a serum concentration of the antibody of greater than about 10 μg/mL for about 2 months.

Embodiment 40. The method of any one of embodiments 1-39, wherein the loading dose maintains a serum concentration of the antibody of greater than about 12 μg/mL for about 3 months.

Embodiment 41. The method of any one of embodiments 1-40, wherein the maintenance dose maintains a serum concentration of the antibody of greater than about 10 μg/mL for about 2 months.

Embodiment 42. The method of any one of embodiments 1-41, wherein the maintenance dose maintains a serum concentration of the antibody of greater than about 12 μg/mL for about 3 months.

Embodiment 43. The method of any one of embodiments 1-42, wherein the maintenance dose is administered about three months after administering the loading dose and then about every three months thereafter.

Embodiment 44. The method of embodiment 43, wherein the loading dose is about 600 mg and the maintenance dose is about 300 mg.

Embodiment 45. The method of embodiment 43, wherein the loading dose is about 400-450 mg and the maintenance dose is about 200-250 mg.

Embodiment 46. The method of any one of embodiments 1-45, wherein the maintenance dose is administered about six months after administering the loading and then about every six months thereafter.

Embodiment 47. The method of embodiment 46, wherein the loading dose is about 1,200 mg and the maintenance dose is about 900 mg.

Embodiment 48. The method of any one of embodiments 1-47, wherein the maintenance dose is administered about 9 months after administering the loading dose and then about every 9 months thereafter.

Embodiment 49. The method of any one of embodiments 1-47, wherein the maintenance doses are administered about every 12 months after the loading dose.

Embodiment 50. A method of treating a human subject having a plasma kallikrein associated disorder, comprising:
administering a first dose of 300-2,000 mg of an anti-plasma kallikrein antibody to the subject; and
after a first period of time from administering the first dose,
(i) administering a second dose of 300-2,000 mg of the anti-plasma kallikrein antibody to the subject and thereafter, at intervals equal to the first period of time, administering one or more subsequent doses of 300-2,000 mg of the anti-plasma kallikrein antibody to the subject; or
(ii) administering a second dose of 300-2,000 mg of an anti-plasma kallikrein antibody to the subject; and after a second period of time equal to the first period of time, administering a third dose of 300-2,000 mg of the anti-plasma kallikrein antibody to the subject and thereafter, at intervals equal to a third period of time that is longer than the first and second periods of time, administering one or more subsequent doses of 300-2,000 mg of the anti-plasma kallikrein antibody to the subject; or
(iii) administering a second dose of 300-2,000 mg of the anti-plasma kallikrein antibody to the subject, and after a second period of time that is longer than the first period of time, administering a third dose of 300-2,000 mg of the anti-plasma kallikrein antibody to the subject and thereafter, at intervals equal to the second period of time, administering one or more subsequent doses of 300-2,000 mg of the anti-plasma kallikrein antibody to the subject;
wherein the first dose, the second dose, any third dose, and any one or more subsequent doses are the same amount; and
wherein the anti-plasma kallikrein antibody comprises:
   i) Complementarity Determining Sequences (CDRs) of a heavy chain variable region having the amino acid sequence of SEQ ID NO: 7, and CDRs of a light chain variable region having the amino acid sequence of SEQ ID NO: 8, wherein the antibody further comprises a constant region comprising an M252Y, an S254T, and a T256E mutation as numbered according to the EU numbering index;
   ii) an immunoglobulin heavy chain variable region comprising the amino acid sequence of SEQ ID NO: 7, and an immunoglobulin light chain variable region comprising the amino acid sequence of SEQ ID NO: 8, wherein the antibody further comprises a constant region comprising an M252Y, an S254T, and a T256E mutation as numbered according to the EU numbering index; or
   iii) an immunoglobulin heavy chain comprising the amino acid sequence of SEQ ID NO: 9 and a light chain comprising the amino acid sequence of SEQ ID NO:10.

Embodiment 51. The method of embodiment 50, wherein after a first period of time from administering the first dose, a second dose of 300-2,000 mg of the anti-plasma kallikrein antibody is administered to the subject and thereafter, at intervals equal to the first period of time, one or more subsequent doses of 300-2,000 mg of the anti-plasma kallikrein antibody are administered to the subject.

Embodiment 52. The method of embodiment 51, wherein the first period of time is between about 3 months and about 9 months.

Embodiment 53. The method of any one of embodiments 51 or 52, wherein the first period of time is selected from about three months, about four months, about five months, about six months, about seven months, about eight months, or about nine months.

Embodiment 54. The method of any one of embodiments 51-53, wherein the first period of time is three months such that the subject receives a dose of the antibody every three months.

Embodiment 55. The method of any one of embodiments 51-53, wherein the first period of time is about four months such that the subject receives a dose of the antibody about every four months.

Embodiment 56. The method of any one of embodiments 51-53, wherein the first period of time is about five months such that the subject receives a dose of the antibody about every five months.

Embodiment 57. The method of any one of embodiments 51-53, wherein the first period of time is about six months such that the subject receives a dose of the antibody about every six months.

Embodiment 58. The method of any one of embodiments 51-53, wherein the first period of time is about seven months such that the subject receives a dose of the antibody about every seven months.

Embodiment 59. The method of any one of embodiments 51-53, wherein the first period of time is about eight months such that the subject receives a dose of the antibody about every eight months.

Embodiment 60. The method of any one of embodiments 51-53, wherein the first period of time is about nine months such that the subject receives a dose of the antibody about every nine months.

Embodiment 61. The method of any one of embodiments 51-53, wherein the first period of time is about twelve months such that the subject receives a dose of the antibody about every twelve months.

Embodiment 62. The method of any one of embodiments 51-61, wherein administration of the first dose, the second dose, or a subsequent dose maintains a serum concentration of the antibody of greater than about 10 μg/mL for at least 2 months, at least 3 months, at least 4 months, at least 5 months, at least 6 months, at least 9 months, or at least 12 months from the administration of the dose.

Embodiment 63. The method of any one of embodiments 51-62, wherein administration of the first dose, the second dose, or a subsequent dose maintains a serum concentration of the antibody of greater than about 12 μg/mL for at least 2 months, at least 3 months, at least 4 months, at least 5 months, at least 6 months, at least 9 months, or at least 12 months from the administration of the dose.

Embodiment 64. The method of any one of embodiments 51-63, wherein administration of the first dose, the second dose, or a subsequent dose maintains a serum concentration of the antibody of greater than about 25 μg/mL for at least 2 months, at least 3 months, at least 4 months, at least 5 months, at least 6 months, at least 9 months, or at least 12 months from the administration of the dose.

Embodiment 65. The method of any one of embodiments 51-64, wherein the first dose is in a range of 300-900 mg.

Embodiment 66. The method of any one of embodiments 51-65, wherein the first period of time is between 3 months and 6 months.

Embodiment 67. The method of any one of embodiments 51-66, wherein the first dose is 700 mg and the first period of time is six months.

Embodiment 68. The method of any one of embodiments 51-64, wherein the first dose is 1,200 mg and the first period of time is six months.

Embodiment 69. The method of any one of embodiments 51-66, wherein the first dose is 600 mg and the first period of time is six months.

Embodiment 70. The method of any one of embodiments 51-66, wherein the first dose is 300-450 mg and the first period of time is three months.

Embodiment 71. The method of embodiment 50, wherein after the first period of time from administering the first dose, a second dose of 300-2,000 mg of an anti-plasma kallikrein antibody is administered to the subject; and after a second period of time equal to the first period of time, a third dose of 300-2,000 mg of the anti-plasma kallikrein antibody is administered to the subject and thereafter, at intervals equal to a third period of time that is longer than the first and second periods of time, one or more subsequent doses of 300-2,000 mg of the anti-plasma kallikrein antibody are administered to the subject.

Embodiment 72. The method of embodiment 71, wherein the first period of time is between about 2 months and about 6 months and the third period of time is between about 4 months and about 9 months.

Embodiment 73. The method of any one of embodiments 71 or 72, wherein the first period of time is selected from about two months, about three months, about four months, about five months, or about six months, and the third period of time is selected from about four months, about five months, about six months, about seven months, about eight months, or about nine months.

Embodiment 74. The method of any one of embodiments 71-73, wherein the first period of time is two months and the third period of time is about four months, such that the subject receives a dose of the antibody about every four months after the third dose.

Embodiment 75. The method of any one of embodiments 71-73, wherein the first period of time is about three months and the third period of time is about six months, such that the subject receives a dose of the antibody about every six months after the third dose.

Embodiment 76. The method of any one of embodiments 71-73, wherein the first period of time is about four months and the third period of time is about eight months, such that the subject receives a dose of the antibody about every eight months after the third dose.

Embodiment 77. The method of any one of embodiments 71-73, wherein the first period of time is about four months and the third period of time is about nine months, such that the subject receives a dose of the antibody about every nine months after the third dose.

Embodiment 78. The method of embodiment 71, wherein the first period of time is about 1-4 weeks.

Embodiment 79. The method of embodiment 78, wherein the first period of time is about 1 week or about 2 weeks.

Embodiment 80. The method of embodiments 78 or 79, wherein the third period of time is about 3 months to about 6 months.

Embodiment 81. The method of embodiments 78-80, wherein the third period of time is 3 months or 6 months.

Embodiment 82. The method of any one of embodiments 71-81, wherein administration of the first dose followed by the second dose, or the third dose, or a subsequent dose maintains a serum concentration of the antibody of greater than about 10 μg/mL for at least 2 months, at least 3 months, at least 4 months, at least 5 months, or at least 6 months from the administration of the dose.

Embodiment 83. The method of any one of embodiments 71-81, wherein administration of the first dose followed by the second dose, or the third dose, or a subsequent dose maintains a serum concentration of the antibody of greater than about 12 μg/mL for at least 2 months, at least 3 months, at least 4 months, at least 5 months, or at least 6 months from the administration of the dose.

Embodiment 84. The method of any one of embodiments 71-81, wherein administration of the first dose followed by the second dose, or the third dose, or a subsequent dose maintains a serum concentration of the antibody of greater than about 25 μg/mL for at least 2 months, at least 3 months, at least 4 months, at least 5 months, or at least 6 months from the administration of the dose.

Embodiment 85. The method of embodiment 50, wherein after the first period of time from administering the first dose, a second dose of 300-2,000 mg of the anti-plasma kallikrein antibody is administered to the subject, and after a second period of time that is longer than the first period of time, a third dose of 300-2,000 mg of the anti-plasma kallikrein antibody is administered to the subject and thereafter, at intervals equal to the second period of time, one or more subsequent doses of 300-2,000 mg of the anti-plasma kallikrein antibody are administered to the subject.

Embodiment 86. The method of embodiment 85, wherein the first period of time is about 1-8 weeks.

Embodiment 87. The method of embodiment 85, wherein the first period of time is about 1-4 weeks.

Embodiment 88. The method of embodiment 85, wherein the first period of time is about 1-2 weeks.

Embodiment 89. The method of embodiment 85, wherein the first period of time is about 1 week.

Embodiment 90. The method of embodiment 85, wherein the first period of time is about 2-6 weeks.

Embodiment 91. The method of embodiment 85, wherein the first period of time is 4 weeks±2 weeks.

Embodiment 92. The method of embodiment 91, wherein the first period of time is about 2 weeks.

Embodiment 93. The method of embodiment 91, wherein the first period of time is about 4 weeks.

Embodiment 94. The method of embodiment 91, wherein the first period of time is about 6 weeks.

Embodiment 95. The method of embodiment 85, wherein the first period of time is about 2 months, 3 months, 4 months, 5 months, or 6 months.

Embodiment 96. The method of embodiment 85 or 95, wherein the first period of time is about 3 months.

Embodiment 97. The method of any one of embodiments 85-96, wherein the second period of time is about 6-12 months.

Embodiment 98. The method of any one of embodiments 85-96, wherein the second period of time is 3-9 months.

Embodiment 99. The method of any one of embodiments 85-96, wherein the second period of time is 3-6 months.

Embodiment 100. The method of any one of embodiments 85-99, wherein the second period of time is about 6 months.

Embodiment 101. The method of any one of embodiments 85-98 wherein the second period of time is about 9 months.

Embodiment 102. The method of any one of embodiments 85-97, wherein the second period of time is about 12 months.

Embodiment 103. The method of any one of embodiments 85-88, 90-94 or 97-100 wherein the second dose is administered 2-6 weeks after the first dose, the third dose is administered about 6 months after the second dose, and subsequent doses are administered at about 6 month intervals after the third dose, such that the subject receives a dose of the antibody about every 6 months after the second dose.

Embodiment 104. The method of embodiment 103, wherein the second dose is administered about 4 weeks after the first dose.

Embodiment 105. The method of any one of embodiments 85-92, 98, or 99, wherein the second dose is administered 1-2 weeks after the first dose, the third dose is administered 3 months after the second dose, and subsequent doses are administered at about 3 months intervals after the third dose, such that the subject receives a dose of the antibody about every 3 months after the second dose.

Embodiment 106. The method of any one of embodiments 85-92, or 97-100, wherein the second dose is administered 1-2 weeks after the first dose, the third dose is administered 6 months after the second dose, and subsequent doses are administered at about 6 months intervals after the third dose, such that the subject receives a dose of the antibody about every 6 months after the second dose.

Embodiment 107. The method of any one of embodiments 85, 95-97, or 102, wherein the second dose is administered about 3 months after the first dose, the third dose is administered about 12 months after the second dose, and subsequent doses are administered at about 12 months intervals after the third dose, such that the subject receives a dose of the antibody about every 12 months after the second dose.

Embodiment 108. The method of any one of embodiments 85-99, wherein the second period of time is about 5 months.

Embodiment 109. The method of any one of embodiments 85, 86, 90-94, or 97-99, wherein the second dose is administered 2-6 weeks after the first dose, the third dose is administered about 5 months after the second dose, and subsequent doses are administered at about 5 month intervals after the third dose, such that the subject receives a dose of the antibody every 5 months after the second dose.

Embodiment 110. The method of embodiment 85, wherein the first period of time is between about 2 months and 6 about months, and the second period of time is between about 4 months and about 9 months.

Embodiment 111. The method of any one of embodiments 85 or 110, wherein the first period of time is selected from about two months, about three months, about four months, about five months, or about six months, and the second period of time is selected from about four months, about five months, about six months, about seven months, about eight months, or about nine months.

Embodiment 112. The method of any one of embodiments 85, 110, or 111, wherein the first period of time is about two months and the second period of time is about four months, such that the subject receives a dose of the antibody about every four months after the second dose.

Embodiment 113. The method of any one of embodiments 85, 110, or 111, wherein the first period of time is about three months and the second period of time is about six months, such that the subject receives a dose of the antibody about every six months after the second dose.

Embodiment 114. The method of any one of embodiments 85, 110, or 111, wherein the first period of time is about four months and the second period of time is about eight months, such that the subject receives a dose of the antibody about every eight months after the second dose.

Embodiment 115. The method of any one of embodiments 85, 110, or 111, wherein the first period of time is about four months and the second period of time is about nine months, such that the subject receives a dose of the antibody about every nine months after the second dose.

Embodiment 116. The method of any one of embodiments 85-115, wherein administration of the first dose, the second dose, the third dose, or a subsequent dose maintains a serum concentration of the antibody of greater than about 10 μg/mL for at least 2 months, at least 3 months, at least 4 months, at least 5 months, at least 6 months, at least 9 months or at least 12 months.

Embodiment 117. The method of any one of embodiments 85-116, wherein administration of the first dose, the second dose, the third dose, or a subsequent dose maintains a serum concentration of the antibody of greater than about 12 μg/mL for at least 2 months, at least 3 months, at least 4 months, at least 5 months, at least 6 months, at least 9 months or at least 12 months.

Embodiment 118. The method of any one of embodiments 85-117, wherein administration of the first dose, the second dose, the third dose, or a subsequent dose maintains a serum concentration of the antibody of greater than or equal to 25 μg/mL for at least 2 months, at least 3 months, at least 4 months, at least 5 months, at least 6 months, at least 9 months or at least 12 months.

Embodiment 119. The method of any one of embodiments 50-118, wherein the first, second, third, and subsequent doses are each about 300-900 mg.

Embodiment 120. The method of any one of embodiments 50-119, wherein the first dose, second dose, third dose and subsequent doses are about 600 mg.

Embodiment 121. The method of any one of embodiments 50-119, wherein the first dose, second dose, third dose and subsequent doses are about 300-450 mg.

Embodiment 122. The method of any one of embodiments 50-119, wherein the first dose, second dose, third dose and subsequent doses are about 900 mg.

Embodiment 123. The method of any one of embodiments 50-118, wherein the first, second, third, and subsequent doses are each about 1,200 mg.

Embodiment 124. The method of any one of embodiments 50-118, wherein the first dose, second dose, any third dose, or any one or more subsequent doses are each 500-1,500 mg.

Embodiment 125. The method of any one of embodiments 50-119, wherein the first dose, second dose, any third dose, and any one or more subsequent doses are selected from one of 500-550 mg, 550-600 mg, 600-650 mg, 650-700 mg, 700-750 mg, 750-800 mg, 850-900 mg, 900-50 mg, 950-1,000 mg, 1,000-1,050 mg, 1,050-1,100 mg, 1,100-1,150 mg, 1,150-1,200 mg, 1,200-1,250 mg, 1,250-1,300 mg, 1,300-1,350 mg, 1,350-1,400 mg, 1,400-1,450 mg, 1,450-1,500 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1,000 mg, 1,025 mg, 1,050 mg, 1,075 mg, 1,100 mg, 1,125 mg, 1,150 mg, 1,175 mg, 1,200 mg, 1,225 mg, 1,250 mg, 1,275 mg, 1,300 mg, 1,325 mg, 1,350 mg, 1,375 mg, 1,400 mg, 1,425 mg, 1,450 mg, 1,475 mg, or 1,500 mg.

Embodiment 126. The method of any one of embodiments 50-119, wherein the first dose, second dose, any third dose, and any one or more subsequent doses are each 600-1,200 mg.

Embodiment 127. The method of any one of embodiments 50-66, 69, 71-118, wherein the first dose, second dose, any third dose, and any one or more subsequent doses are each 600 mg.

Embodiment 128. The method of any one of embodiments 50-66, or 71-118, wherein the first dose, second dose, any third dose, and any one or more subsequent doses are each 900 mg.

Embodiment 129. The method of any one of embodiments 50-66, 68, or 71-118, wherein the first dose, second dose, any third dose, and any one or more subsequent doses are each 1,200 mg.

Embodiment 130. The method of any one of embodiments 50-129, wherein the first dose, second dose, any third dose, and any one or more subsequent doses are administered to the subject subcutaneously.

Embodiment 131. The method of any one of embodiments 50-129, wherein the first dose, second dose, any third dose, and any one or more subsequent doses are administered to the subject intravenously.

Embodiment 132. The method of any one of embodiments 50-129, wherein the first dose is administered intravenously and the second dose, any third dose and any one or more subsequent doses are administered subcutaneously.

Embodiment 133. The method of any one of embodiments 50-129, wherein the first and second doses are administered intravenously and any third dose and any one or more subsequent doses are administered subcutaneously.

Embodiment 134. The method of any one of embodiments 50-129, wherein the first dose, second dose, any third dose and any one or more subsequent doses are administered to the subject using a combination of intravenous and subcutaneous administration.

Embodiment 135. The method of any one of embodiments 130, 132, or 133, wherein the dose or doses administered subcutaneously are administered from a prefilled syringe, autoinjector device, or via an on-body or wearable injection device.

Embodiment 136. The method of any one of embodiments 131, 133, or 134, wherein the dose or doses administered intravenously are administered as a bolus injection or by infusion.

Embodiment 137. A method of treating a human subject having a plasma kallikrein associated disorder, comprising:
administering a first dose of 300-2,000 mg of an anti-plasma kallikrein antibody to the subject, followed by administering one or more treatment cycles consisting of a first subsequent dose of 300-2,000 mg of the antibody followed by a second subsequent dose of 300-2,000 mg of the antibody,
wherein the first subsequent dose is administered after a first period of time from the first dose, or after a third period of time from the second subsequent dose of a previous treatment cycle, wherein the third period of time is equal to the first period of time; and the second subsequent dose is administered after a second period of time from the first subsequent dose;
wherein the first period of time is shorter than the second period of time;
wherein the first dose, the first subsequent dose, and the second subsequent doses are the same amount; and
wherein the anti-plasma kallikrein antibody comprises:
   i) Complementarity Determining Sequences (CDRs) of a heavy chain variable region having the amino acid sequence of SEQ ID NO: 7, and CDRs of a light chain variable region having the amino acid sequence of SEQ ID NO: 8, wherein the antibody further comprises a constant region comprising an M252Y, an S254T, and a T256E mutation as numbered according to the EU numbering index;
   ii) an immunoglobulin heavy chain variable region comprising the amino acid sequence of SEQ ID NO: 7, and an immunoglobulin light chain variable region comprising the amino acid sequence of SEQ ID NO: 8, wherein the antibody further comprises a constant region comprising an M252Y, an S254T, and a T256E mutation as numbered according to the EU numbering index; or
   iii) an immunoglobulin heavy chain comprising the amino acid sequence of SEQ ID NO: 9 and a light chain comprising the amino acid sequence of SEQ ID NO:10.

Embodiment 138. The method of embodiment 137, wherein the first period of time is about 2-6 weeks.

Embodiment 139. The method of any one of embodiments 137 or 138, wherein the first period of time is about 2 weeks, 3 weeks, 4 weeks, 5 weeks, or 6 weeks.

Embodiment 140. The method of any one of embodiments 137 or 138, wherein the first period of time is 4 weeks±2 weeks.

Embodiment 141. The method of any one of embodiments 137-140. wherein the first period of time is about 2 weeks.

Embodiment 142. The method of any one of embodiments 137-140, wherein the first period of time is about 4 weeks.

Embodiment 143. The method of any one of embodiments 137-140, wherein the first period of time is about 6 weeks.

Embodiment 144. The method of any one of embodiments 137-140, wherein the second period of time is 3-9 months.

Embodiment 145. The method of any one of embodiments 137-144, wherein the second period of time is about 3 months, about 4 months, about 5 months, about 6 months, about 7 months, about 8 months, or about 9 months.

Embodiment 146. The method of any one of embodiments 137-144, wherein the second period of time is 3 months±2 weeks, 4 months±2 weeks, 5 months±2 weeks, 6 months±2 weeks, 7 months±2 weeks, 8 months±2 weeks, or 9 months±2 weeks.

Embodiment 147. The method of any one of embodiments 137-144, wherein the second period of time is about 6 months.

Embodiment 148. The method of any one of embodiments 137-144, wherein the second period of time is 6 months±2 weeks

Embodiment 149. The method of any one of embodiments 137-138, wherein (a) the first subsequent dose is administered 2-6 weeks after the first dose, or 2-6 weeks after the second subsequent dose of a previous treatment cycle, and (b) the second subsequent dose is administered 3-9 months after the first subsequent dose.

Embodiment 150. The method of any one of embodiments 137-138, wherein (a) the first subsequent dose is administered 4 weeks±2 weeks after the first dose, or 4 weeks±2 weeks after the second subsequent dose of a previous treatment cycle, and (b) the second subsequent dose is administered 6 months±2 weeks after the first subsequent dose.

Embodiment 151. The method of any one of embodiments 137-138, wherein (a) the first subsequent dose is administered 2 weeks after the first dose, or 2 weeks after the second subsequent dose of a previous treatment cycle, and (b) the second subsequent dose is administered 6 months±2 weeks after the first subsequent dose.

Embodiment 152. The method of any one of embodiments 137-138, wherein (a) the first subsequent dose is administered 4 weeks after the first dose, or 4 weeks after the second subsequent dose of a previous treatment cycle, and (b) the second subsequent dose is administered 6 months±2 weeks after the first subsequent dose.

Embodiment 153. The method of any one of embodiments 149-152, wherein the second subsequent dose is administered 6 months after the first subsequent dose.

Embodiment 154. The method of any one of embodiments 137-145, wherein the second period of time is about 5 months.

Embodiment 155. The method of any one of embodiments 137-146, wherein the second period of time is 5 months±2 weeks.

Embodiment 156. The method of embodiment 137, wherein (a) the first subsequent dose is administered 4 weeks±2 weeks after the first dose, or 4 weeks±2 weeks after the second subsequent dose of a previous treatment cycle, and (b) the second subsequent dose is administered 5 months±2 weeks after the first subsequent dose.

Embodiment 157. The method of embodiment 137, wherein (a) the first subsequent dose is administered 2 weeks after the first dose, or 2 weeks after the second subsequent dose of a previous treatment cycle, and (b) the second subsequent dose is administered 5 months=2 weeks after the first subsequent dose.

Embodiment 158. The method of embodiment 137, wherein (a) the first subsequent dose is administered 4 weeks after the first dose, or 4 weeks after the second subsequent dose of a previous treatment cycle, and (b) the second subsequent dose is administered 5 months±2 weeks after the first subsequent dose.

Embodiment 159. The method of any one of embodiments 156-158, wherein the second subsequent dose is administered 5 months after the first subsequent dose.

Embodiment 160. The method of any one of embodiments 137-159, wherein administration of the first dose followed by a first subsequent dose maintains a serum concentration of the antibody of greater than about 10 μg/mL for at least about 5 months from the time of administration of the first dose, or administration of a second subsequent dose followed by a first subsequent dose maintains a serum concentration of the antibody of greater than about 10 μg/mL for at least about 5 months from the time of administration of the second subsequent dose.

Embodiment 161. The method of embodiment 160, wherein the serum concentration is maintained at greater than or equal to 12 μg/mL for 5 months.

Embodiment 162. The method of embodiment 161, wherein the serum concentration is maintained at greater than or equal to 25 μg/mL for 5 months.

Embodiment 163. The method of any one of embodiments 137-162, wherein the first dose, the first subsequent dose and the second subsequent dose are each 500-1,500 mg.

Embodiment 164. The method of any one of embodiments 137-163, wherein the first dose, the first subsequent dose and the second subsequent dose are selected from one of 500-550 mg, 550-600 mg, 600-650 mg, 650-700 mg, 700-750 mg, 750-800 mg, 850-900 mg, 900-50 mg, 950-1,000 mg, 1,000-1,050 mg, 1,050-1,100 mg, 1,100-1,150 mg, 1, 150-1,200 mg, 1,200-1,250 mg, 1,250-1,300 mg, 1,300-1,350 mg, 1,350-1,400 mg, 1,400-1,450 mg, 1,450-1,500 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1,000 mg, 1,025 mg, 1,050 mg, 1,075 mg, 1,100 mg, 1,125 mg, 1,150 mg, 1,175 mg, 1,200 mg, 1,225 mg, 1,250 mg, 1,275 mg, 1,300 mg, 1,325 mg, 1,350 mg, 1,375 mg, 1,400 mg, 1,425 mg, 1,450 mg, 1,475 mg, or 1,500 mg.

Embodiment 165. The method of any one of embodiments 137-164, wherein the first dose, the first subsequent dose and the second subsequent dose are each 600-1,200 mg.

Embodiment 166. The method of any one of embodiments 137-165, wherein the first dose, the first subsequent dose and the second subsequent dose are each 600 mg.

Embodiment 167. The method of embodiment 166, wherein the first period of time is 1 month and the second period of time is 6 months.

Embodiment 168. The method of any one of embodiments 137-165, wherein the first dose, the first subsequent dose and the second subsequent dose are each 900 mg.

Embodiment 169. The method of embodiment 168, wherein the first period of time is 1 month and the second period of time is 6 months.

Embodiment 170. The method of any one of embodiments 137-165, wherein the first dose, the first subsequent dose and the second subsequent dose are each 1,200 mg.

Embodiment 171. The method of any one of embodiments 137-170, wherein the first dose, first subsequent dose and second subsequent dose are administered to the subject subcutaneously.

Embodiment 172. The method of any one of embodiments 137-170, wherein the first dose, first subsequent dose and second subsequent dose are administered to the subject intravenously.

Embodiment 173. The method of any one of embodiments 137-170, wherein the first dose and the first subsequent dose are administered to the subject intravenously and the second subsequent dose is administered subcutaneously.

Embodiment 174. The method of any one of embodiments 137-170, wherein the first dose, first subsequent dose, and second subsequent dose are administered to the subject using a combination of intravenous and subcutaneous administration.

Embodiment 175. The method of any one of embodiments 171, 173, or 174, wherein the dose or doses administered subcutaneously are administered from a prefilled syringe, autoinjector device, or via an on-body or wearable injection device.

Embodiment 176. The method of any one of embodiments 172-174, wherein the dose or doses administered intravenously are administered as a bolus injection or by infusion.

Embodiment 177. A method of treating a human subject having a plasma kallikrein associated disorder comprising:
administering treatment cycles of an anti-plasma kallikrein antibody to the subject, wherein a treatment cycle comprises administering a first dose of 300-2,000 mg of the anti-plasma kallikrein antibody to the subject, then after a first period of time from administering the first dose, administering a second dose of 300-2,000 mg of an anti-plasma kallikrein antibody to the subject, then after a second period of time from the second dose, repeating administration of the treatment cycle one or more times, wherein the second period of time is longer than the first period of time, wherein the first dose and the second dose are the same; and
wherein the anti-plasma kallikrein antibody comprises:
   i) Complementarity Determining Sequences (CDRs) of a heavy chain variable region having the amino acid sequence of SEQ ID NO: 7, and CDRs of a light chain variable region having the amino acid sequence of SEQ ID NO: 8, wherein the antibody further comprises a constant region comprising an M252Y, an S254T, and a T256E mutation as numbered according to the EU numbering index;
   ii) an immunoglobulin heavy chain variable region comprising the amino acid sequence of SEQ ID NO: 7, and an immunoglobulin light chain variable region comprising the amino acid sequence of SEQ ID NO: 8, wherein the antibody further comprises a constant region comprising an M252Y, an S254T, and a T256E mutation as numbered according to the EU numbering index; or
   iii) an immunoglobulin heavy chain comprising the amino acid sequence of SEQ ID NO: 9 and a light chain comprising the amino acid sequence of SEQ ID NO:10.

Embodiment 178. The method of embodiment 177, wherein the first period of time is 2-6 weeks.

Embodiment 179. The method of any one of embodiments 177-178, wherein the first period of time is about 2 weeks, about 3 weeks, about 4 weeks, about 5 weeks, or about 6 weeks.

Embodiment 180. The method of any one of embodiments 177-179, wherein the first period of time is 4 weeks±2 weeks.

Embodiment 181. The method of any one of embodiments 177-180, wherein the first period of time is about 2 weeks.

Embodiment 182. The method of any one of embodiments 177-180, wherein the first period of time is about 4 weeks.

Embodiment 183. The method of any one of embodiments 177-180, wherein the first period of time is about 6 weeks.

Embodiment 184. The method of any one of embodiments 177-183, wherein the second period of time is 3-9 months.

Embodiment 185. The method of any one of embodiments 177-184, wherein the second period of time is about 3 months, about 4 months, about 5 months, about 6 months, about 7 months, about 8 months, or about 9 months.

Embodiment 186. The method of any one of embodiments 177-184, wherein the second period of time is 3 months±2 weeks, 4 months±2 weeks, 5 months±2 weeks, 6 months±2 weeks, 7 months±2 weeks, 8 months±2 weeks, or 9 months±2 weeks.

Embodiment 187. The method of any one of embodiments 177-185, wherein the second period of time is about 6 months.

Embodiment 188. The method of any one of embodiments 177-186, wherein the second period of time is 6 months±2 weeks.

Embodiment 189. The method of any one of embodiments 177-188, wherein the second dose is administered 2-6 weeks after the first dose and the treatment cycle is repeated 6 months±2 weeks after the second dose.

Embodiment 190. The method of any one of embodiments 177-188, wherein the second dose is administered 4 weeks±2 weeks after the first dose and the treatment cycles is repeated 6 months±2 weeks after the second dose.

Embodiment 191. The method of any one of embodiments 177-188, wherein the second dose is administered 2 weeks after the first dose and the treatment cycle is repeated 6 months±2 weeks after the second dose.

Embodiment 192. The method of any one of embodiments 177-188, wherein the second dose is administered 4 weeks after the first dose, and the treatment cycle is repeated 6 months±2 weeks after the second dose.

Embodiment 193. The method of any one of embodiments 188-192, wherein the treatment cycle is repeated 6 months after the second dose

Embodiment 194. The method of any one of embodiments 177-188, wherein the second dose is administered 2-6 weeks after the first dose and the treatment cycle is repeated 6 months after the first dose, such that the first period of time plus the second period of time equals 6 months.

Embodiment 195. The method of any one of embodiments 177-188, wherein the second dose is administered 4 weeks±2 weeks after the first dose and the treatment cycles is repeated 6 months after the first dose, such that the first period of time plus the second period of time equals 6 months.

Embodiment 196. The method of any one of embodiments 177-188, wherein the second dose is administered 2 weeks after the first dose and the treatment cycle is repeated 6 months after the first dose, such that the first period of time plus the second period of time equals 6 months.

Embodiment 197. The method of any one of embodiments 177-188, wherein the second dose is administered 4 weeks after the first dose, and the treatment cycle is repeated 6 months after the first dose, such that the first period of time plus the second period of time equals 6 months.

Embodiment 198. The method of any one of embodiments 177-197, wherein the second period of time is 5 months.

Embodiment 199. The method of any one of embodiments 177-197, wherein the second period of time is 5 months±2 weeks.

Embodiment 200. The method of embodiment 177, wherein the second dose is administered 2-6 weeks after the first dose and the treatment cycle is repeated 5 months±2 weeks after the second dose.

Embodiment 201. The method of embodiment 177, wherein the second dose is administered 4 weeks±2 weeks after the first dose and the treatment cycles is repeated 5 months±2 weeks after the second dose.

Embodiment 202. The method of embodiment 177, wherein the second dose is administered 2 weeks after the first dose and the treatment cycle is repeated 5 months±2 weeks after the second dose.

Embodiment 203. The method of embodiment 177, wherein the second dose is administered 4 weeks after the first dose, and the treatment cycle is repeated 5 months±2 weeks after the second dose.

Embodiment 204. The method of any one of embodiments 200-203, wherein the second dose is administered 5 months after the second dose.

Embodiment 205. The method of embodiment 177, wherein the second dose is administered 2-6 weeks after the first dose and the treatment cycle is repeated 7 months after the first dose, such that the first period of time plus the second period of time equals 7 months.

Embodiment 206. The method of embodiment 177, wherein the second dose is administered 4 weeks±2 weeks after the first dose and the treatment cycle is repeated 7 months after the first dose, such that the first period of time plus the second period of time equals 7 months.

Embodiment 207. The method of embodiment 177, wherein the second dose is administered 2 weeks after the first dose and the treatment cycle is repeated 7 months after the first dose, such that the first period of time plus the second period of time equals 7 months.

Embodiment 208. The method of embodiment 177, wherein the second dose is administered 4 weeks after the first dose, and the treatment cycle is repeated 7 months after the first dose, such that the first period of time plus the second period of time equals 7 months.

Embodiment 209. The method of embodiment 177, wherein the first period of time is 1-2 weeks.

Embodiment 210. The method of embodiment 209, wherein the second period of time is about 3-6 months.

Embodiment 211. The method of embodiment 209, wherein the second period of time is 6 months±2 weeks.

Embodiment 212. The method of embodiment 209, wherein the second period of time is 3 months±2 weeks.

Embodiment 213. The method of embodiment 210 or 211, wherein the first period of time plus the second period of time totals 6 months.

Embodiment 214. The method of embodiment 210 or 212, wherein the first period of time plus the second period of time totals 3 months.

Embodiment 215. The method of any one of embodiments 177-214, wherein administration of a treatment cycle maintains a serum concentration of the antibody of greater than about 10 μg/mL for at least about 5 months from the time of administration of the first dose.

Embodiment 216. The method of embodiment 215, wherein the serum concentration is maintained at greater than or equal to 12 μg/mL for at least 5 months from the administration of the first dose.

Embodiment 217. The method of embodiment 215 or 216, wherein the serum concentration is maintained at greater than or equal to 25 μg/mL for at least 5 months from the administration of the first dose.

Embodiment 218. The method of embodiments 215-217, wherein the serum concentration is maintained for at least 6 months from administration of the first dose.

Embodiment 219. The method of any one of embodiments 177-218, wherein the first dose and the second dose are each 500-1,500 mg.

Embodiment 220. The method of any one of embodiments 177-218, wherein the first dose and the second dose are each 300-990 mg.

Embodiment 221. The method of any one of embodiments 177-218, wherein the first dose and the second dose are each 600-1,200 mg.

Embodiment 222. The method of any one of embodiments 177-221, wherein the first dose and the second dose are selected from one of 500-550 mg, 550-600 mg, 600-650 mg, 650-700 mg, 700-750 mg, 750-800 mg, 850-900 mg, 900-50 mg, 950-1,000 mg, 1,000-1,050 mg, 1,050-1,100 mg, 1,100-1,150 mg, 1,150-1,200 mg, 1,200-1,250 mg, 1,250-1,300 mg, 1,300-1,350 mg, 1,350-1,400 mg, 1,400-1,450 mg, 1,450-1,500 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1,000 mg, 1,025 mg, 1,050 mg, 1,075 mg, 1,100 mg, 1,125 mg, 1,150 mg, 1,175 mg, 1,200 mg, 1,225 mg, 1,250 mg, 1,275 mg, 1,300 mg, 1,325 mg, 1,350 mg, 1,375 mg, 1,400 mg, 1,425 mg, 1,450 mg, 1,475 mg, or 1,500 mg.

Embodiment 223. The method of any one of embodiments 177-222, wherein the first dose and the second dose are each about 600 mg.

Embodiment 224. The method of any one of embodiments 177-222, wherein the first dose and the second dose are each about 900 mg.

Embodiment 225. The method of any one of embodiments 177-219, or 221-222, wherein the first dose and the second dose are each about 1,200 mg.

Embodiment 226. The method of any one of embodiments 177-225, wherein the first dose and the second dose are administered to the subject subcutaneously.

Embodiment 227. The method of any one of embodiments 177-226 wherein the first dose and the second dose are administered to the subject intravenously.

Embodiment 228. The method of any one of embodiments 177-227, wherein the first dose is administered to the subject intravenously and the second dose is administered subcutaneously.

Embodiment 229. The method of any one of embodiments 226 or 228, wherein the dose administered subcutaneously is administered from a prefilled syringe, autoinjector device, or via an on-body or wearable injection device.

Embodiment 230. The method of any one of embodiments 227 or 228, wherein the dose administered intravenously is administered as a bolus injection or by infusion.

Embodiment 231. The method of any one of embodiments 1-230, wherein the plasma kallikrein associated disorder is hereditary angioedema (HAE).

Embodiment 232. The method of any one of embodiments 1-231, wherein the antibody comprises Complementarity Determining Sequences (CDRs) of a heavy chain variable region having the amino acid sequence of SEQ ID NO: 7, and CDRs of a light chain variable region having the amino acid sequence of SEQ ID NO: 8, wherein the antibody further comprises a constant region comprising an M252Y, an S254T, and a T256E mutation as numbered according to the EU numbering index.

Embodiment 233. The method of any one of embodiments 1-232 wherein the antibody comprises an immunoglobulin heavy chain variable region comprising a CDRH1 comprising the amino acid sequence of SEQ ID NO: 1, a CDRH2 comprising the amino acid sequence of SEQ ID NO: 2, and a CDRH3 comprising the amino acid sequence of SEQ ID NO: 3; and an immunoglobulin light chain variable region comprising a CDRL1 comprising the amino acid sequence of SEQ ID NO: 4, a CDRL2 comprising the amino acid sequence of SEQ ID NO: 5, and a CDRL3 comprising the amino acid sequence of SEQ ID NO: 6; and wherein the antibody further comprises a constant region comprising an M252Y, an S254T, and a T256E mutation as numbered according to the EU numbering index.

Embodiment 234. The method of any one of embodiments 1-233, wherein the antibody comprises an immunoglobulin heavy chain variable region comprising the amino acid sequence of SEQ ID NO: 7, and an immunoglobulin light chain variable region comprising the amino acid sequence of SEQ ID NO: 8, wherein the antibody further comprises a constant region comprising an M252Y, an S254T, and a T256E mutation as numbered according to the EU numbering index

Embodiment 235. The method of any one of embodiments 1-234 wherein the antibody comprises an immunoglobulin heavy chain comprising the amino acid sequence of SEQ ID NO: 9 and a light chain comprising the amino acid sequence of SEQ ID NO:10.

Embodiment 236. The method of any one of embodiments 1-235, wherein the antibody comprises a IgG1 constant domain.

Embodiment 237. The method of any one of embodiments 1-236, wherein the anti-plasma kallikrein antibody is administered to the subject at a concentration of 100-200 mg/mL.

Embodiment 238. The method of any one of embodiments 1-237, wherein the anti-plasma kallikrein antibody is administered to the subject at a concentration of 100-150 mg/mL or 150-200 mg/mL.

Embodiment 239. The method of any one of embodiments 1-238, wherein the anti-plasma kallikrein antibody is administered to the subject at a concentration of 100 mg/mL, 150 mg/mL, or 200 mg/mL.

Embodiment 240. The method of any one of embodiments 1-239, wherein the subject experiences a reduction in the number of HAE attacks per month as compared to the subject's baseline number of attacks per month prior to beginning treatment.

Embodiment 241. The method of embodiment 240, wherein the reduction from baseline is 75-90% or 75-100%.

Embodiment 242. The method of any one of embodiments 1-241, wherein the method reduces the severity of an HAE attack in the subject as compared to the subject's baseline severity of attacks prior to beginning treatment.

Embodiment 243. The method of any one of embodiments 1-242, wherein the method reduces the subject's Angioedema Quality of Life Score (AEQoL) by at least 6 points, at least 10 points, at least 15 points, or at least 20 points as compared to the subject's AEQoL score prior to beginning treatment.

Embodiment 244. The method of any one of embodiments 1-243, wherein administration of the antibody reduces the level of activated plasma kallikrein in the subject compared to the subject's baseline prior to beginning treatment, optionally wherein the level of plasma kallikrein is measured about once per month.

Embodiment 245. The method of any one of embodiments 1-244, wherein the anti-plasma kallikrein antibody is formulated in a pharmaceutical composition with a pharmaceutically acceptable excipient.

Embodiment 246. The method of any one of embodiments 1-245, wherein the pharmaceutical composition comprises histidine, L-methionine, sorbitol, glycine, and polysorbate 80.

Embodiment 247. The method of any one of embodiments 1-246, wherein the pharmaceutical composition comprises 10 mM histidine-HCl, 75 mM glycine, 10 mM L-methionine, 150 mM sorbitol, and 0.05% w/v polysorbate 80 at pH 5.8.

Embodiment 248. The method of any one of embodiments 1-247, wherein the anti-plasma kallikrein antibody is STAR-0215.

Embodiment 249. The method of any one of embodiments 1-230, or 232-248, wherein the plasma kallikrein associated disorder is selected from hereditary angioedema (including Type I, II, or III), bradykinin dependent edema, diabetic macular edema, retinal edema, Factor XII-associated cold autoinflammatory syndrome (FACAS), rheumatoid arthritis, gout, intestinal bowel disease, oral mucositis, neuropathic pain, inflammatory pain, spinal stenosis-degenerative spine disease, arterial or venous thrombosis, post-operative ileus, aortic aneurysm, osteoarthritis, vasculitis, edema, cerebral edema, pulmonary embolism, stroke, clotting induced by ventricular assistance devices or stents, head trauma or peri-tumor brain edema, sepsis, acute middle cerebral artery (MCA) ischemic event (stroke), restenosis (e.g., after angioplasty), systemic lupus erythematosus nephritis, Alzheimer's disease, and burn injury.

1. A method of reducing the number of hereditary angioedema (HAE) attacks that a subject with HAE experiences per month (monthly attack rate), the method comprising treating the subject with an anti-plasma kallikrein antibody comprising two heavy chains and two light chains, wherein each heavy chain comprises the amino acid sequence of SEQ ID NO:9 and each light chain comprises the amino acid sequence of SEQ ID NO: 10 according to a dosing regimen comprising one of:
(a) administering a loading dose of 600 mg of the antibody followed by administering a maintenance dose of 300 mg of the antibody about every three months after the loading dose;
(b) administering a first dose of 600 mg of the antibody followed by administering a second dose of 600 mg of the antibody about 28 days after the first dose, and then administering 600 mg of the antibody about every six months after the second dose; or
(c) administering a dose of at least 450 mg of the antibody to the subject every six months;
wherein the treatment reduces the monthly attack rate for the subject from the subject's monthly attack rate prior to initiation of the treatment by at least 85% for at least 3 months from initiation of the dosing regimen.

2-4. (canceled)

5. The method of claim 1, wherein the treatment reduces the subject's monthly HAE attack rate from the subject's baseline by at least about 85%, about 86%, about 87%, about 88%, about 89%, about 90%, about 91%, about 92%, about 93%, about 94%, about 95%, about 96%, about 97%, about 98%, about 99%, or by 100%.

6. The method of claim 1, wherein the treatment reduces the monthly attack rate for the subject for at least 6 months from initiation of the dosing regimen.

7. The method of claim 1, wherein the subject is attack free for (i) at least three months from initiation of the dosing regimen and/or (ii) at least six months from initiation of the dosing regimen.

8. (canceled)

9. A method of reducing the severity and/or duration of hereditary angioedema (HAE) attacks in a subject with HAE, the method comprising treating the subject with an anti-plasma kallikrein antibody comprising two heavy chains and two light chains, wherein each heavy chain comprises the amino acid sequence of SEQ ID NO:9 and each light chain comprises the amino acid sequence of SEQ ID NO: 10 according to a dosing regimen comprising one of:
(a) administering a loading dose of 600 mg of the antibody followed by administering a maintenance dose of 300 mg of the antibody about every three months after the loading dose;
(b) administering a first dose of 600 mg of the antibody followed by administering a second dose of 600 mg of the antibody about 28 days after the first dose, and then administering 600 mg of the antibody about every six months after the second dose; or
(c) administering a dose of at least 450 mg of the antibody to the subject every six months;
wherein the treatment (i) reduces the monthly rate of moderate or severe HAE attacks the subject experiences by at least 85% for at least 3 months from initiation of the dosing regimen as compared to the monthly rate of moderate or severe HAE attacks prior to initiation of the treatment, or (ii) reduces the average duration of HAE attacks the subject experiences for at least three months from initiation of the dosing regimen as compared to average duration of HAE attacks in the subject prior to initiation of the treatment.

10. The method of claim 9, wherein the treatment reduces the monthly rate of moderate or severe HAE attacks the subject experiences by (i) at least 90% for at least 3 months from initiation of the dosing regimen as compared to the monthly rate of moderate or severe HAE attacks in the subject prior to initiation of the treatment and/or (ii) by at least 85% for at least 6 months from initiation of the dosing regimen as compared to the monthly rate of moderate or severe HAE attacks in the subject prior to initiation of the treatment.

11. (canceled)

12. The method of claim 9, wherein the treatment reduces the subject's monthly rate of moderate and severe attacks from the subject's baseline by at least about 85%, about 86%, about 87%, about 88%, about 89%, about 90%, about 91%, about 92%, about 93%, about 94%, about 95%, about 96%, about 97%, about 98%, about 99%, or by 100%.

13. The method of claim 9, wherein the subject (i) does not experience any severe HAE attacks for at least 3 months from initiation of the dosing regimen and/or (ii) does not experience any severe HAE attacks for at least 6 months from initiation of the dosing regimen; and/or (iii) the subject does not experience any moderate HAE attacks for at least 3 or 6 months from initiation of the dosing regimen.

14-15. (canceled)

16. The method of claim 9, wherein the treatment reduces the average duration of HAE attacks the subject experiences (i) for at least three months from initiation of the dosing regimen as compared to the duration of HAE attacks in the subject prior to initiation of the treatment and/or (ii) for at least six months from initiation of the dosing regimen as compared to the duration of HAE attacks in the subject prior to initiation of the treatment.

17. (canceled)

18. A method of reducing the need for a subject with hereditary angioedema (HAE) to receive on-demand therapy (rescue medication) when experiencing an HAE attack, the method comprising treating the subject with an anti-plasma kallikrein antibody comprising two heavy chains and two light chains, wherein each heavy chain comprises the amino acid sequence of SEQ ID NO:9 and each light chain comprises the amino acid sequence of SEQ ID NO:10 according to a dosing regimen comprising one of:
(a) administering a loading dose of 600 mg of the antibody followed by administering a maintenance dose of 300 mg of the antibody about every three months after the loading dose;
(b) administering a first dose of 600 mg of the antibody followed by administering a second dose of 600 mg of the antibody about 28 days after the first dose, and then administering 600 mg of the antibody about every six months after the second dose; or
(c) administering a dose of at least 450 mg of the antibody to the subject every six months;
wherein the treatment reduces the subject's monthly attack rate for which the subject receives on-demand therapy by at least 85% compared to the subject's monthly attack rate for which the subject received on-demand therapy prior to initiation of the treatment, wherein the reduction lasts for at least 3 months from initiation of the dosing regimen.

19. The method of claim 17, wherein the reduction lasts for at least 6 months from initiation of the dosing regimen.

20. The method of claim 18, wherein the treatment reduces the subject's monthly attack rate for which the subject receives on-demand HAE therapy from the subject's monthly attack rate by at least about 85%, about 86%, about 87%, about 88%, about 89%, about 90%, about 91%, about 92%, about 93%, about 94%, about 95%, about 96%, about 97%, about 98%, about 99%, or by 100%.

21. The method of claim 18, wherein the subject does not experience any HAE attacks requiring receipt of on-demand HAE therapy for (i) at least three months from initiation of the dosing regimen and/or (ii) for at least six months from initiation of the dosing regimen.

22. (canceled)

23. A method of maintaining a serum concentration of greater than about 12 μg/mL of an anti-plasma kallikrein antibody in subject with hereditary angioedema (HAE), the method comprising administering to the subject an anti-plasma kallikrein antibody comprising two heavy chains and two light chains, wherein each heavy chain comprises the amino acid sequence of SEQ ID NO:9 and each light chain comprises the amino acid sequence of SEQ ID NO: 10, wherein the antibody is administered according to one of the following dosing regimens:
(a) administering a loading dose of 600 mg followed by administering a maintenance dose of 300 mg three months after the loading dose;
(b) administering a first dose of 600 mg followed by administering a second dose of 600 mg about 28 days after the first dose; or
(c) administering at least 450 mg of the antibody to the subject every six months;
wherein the serum concentration exceeds 12 μg/mL for at least three months from a loading dose or first dose of the antibodies.

24. The method of claim 23, wherein the dosing regimen comprises:
(i) administering a loading dose of 600 mg followed by administering a maintenance dose of 300 mg about every three months after the loading dose, wherein the serum concentration exceeds 12 μg/mL for at least three months from the loading dose, and/or for at least three months from the maintenance dose of the antibody;
(ii) administering a first dose of 600 mg followed by administering a second dose of 600 mg about 28 days after the first dose, and then administering 600 mg every six months after the second dose, wherein the serum concentration is exceeds 12 μg/mL for at least 6 months from administration of the first dose, or for at least 6 months from administration of the second dose, or a subsequent 600 mg dose; or
(iii) administering a dose of least 450 mg of the antibody to the subject every six months, and the serum concentration exceeds 12 μg/mL for at least 6 months from administration of the dose.

25-26. (canceled)

27. The method of claim 1, wherein (i) the antibody is administered to the subject via subcutaneous injection and/or (ii) wherein the antibody is STAR-0215.

28. (canceled)

29. The method of claim 1 wherein
(i) the dose of at least 450 mg is in a range of 450 mg-1,200 mg;
(ii) the dose of at least 450 mg is in a range of 450 mg-900 mg;
(iii) the dose of at least 450 mg is in a range of 450 mg-600 mg;
(iv) an initial dose of at least 450 mg is preceded by a higher loading dose less than 6 months before administration of the initial dose; and/or
(v) an initial dose of at least 450 mg is preceded by the same dose less than 6 months before administration of the initial dose.

30-33. (canceled)

34. The method of claim 9, wherein (i) the antibody is administered to the subject via subcutaneous injection and/or (ii) wherein the antibody is STAR-0215.

35. The method of claim 18, wherein (i) the antibody is administered to the subject via subcutaneous injection and/or (ii) wherein the antibody is STAR-0215.

36. The method of claim 23, wherein (i) the antibody is administered to the subject via subcutaneous injection and/or (ii) wherein the antibody is STAR-0215.

37. The method claim 9, wherein:
(i) the dose of at least 450 mg is in a range of 450 mg-1,200 mg;
(ii) the dose of at least 450 mg is in a range of 450 mg-900 mg;
(iii) the dose of at least 450 mg is in a range of 450 mg-600 mg;
(iv) an initial dose of at least 450 mg is preceded by a higher loading dose less than 6 months before administration of the initial dose; and/or
(v) an initial dose of at least 450 mg is preceded by the same dose less than 6 months before administration of the initial dose.

38. The method claim 18, wherein:
(i) the dose of at least 450 mg is in a range of 450 mg-1,200 mg;
(ii) the dose of at least 450 mg is in a range of 450 mg-900 mg;
(iii) the dose of at least 450 mg is in a range of 450 mg-600 mg;
(iv) an initial dose of at least 450 mg is preceded by a higher loading dose less than 6 months before administration of the initial dose; and/or
(v) an initial dose of at least 450 mg is preceded by the same dose less than 6 months before administration of the initial dose.

39. The method claim 23, wherein:
(i) the dose of at least 450 mg is in a range of 450 mg-1,200 mg;
(ii) the dose of at least 450 mg is in a range of 450 mg-900 mg;
(iii) the dose of at least 450 mg is in a range of 450 mg-600 mg;
(iv) an initial dose of at least 450 mg is preceded by a higher loading dose less than 6 months before administration of the initial dose; and/or
(v) an initial dose of at least 450 mg is preceded by the same dose less than 6 months before administration of the initial dose.
